(19) United States
(12) Patent Application Publication
(10) Pub. No.: US 2026/0248916 A1
(43) Pub. Date: Aug. 27, 2026

(54) INDUCING INTERMEDIATE FILAMENT DISASSEMBLY

(71) Applicant: HOWARD HUGHES MEDICAL INSTITUTE, Chevy Chase, MD (US)

(72) Inventors: Andrew Moore, Arlington, VA (US); Thomas Krug, Cambridge, MA (US)

(73) Assignees: HOWARD HUGHES MEDICAL INSTITUTE, Chevy Chase, MD (US); PRESIDENT AND FELLOWS OF HARVARD COLLEGE, Cambridge, MA (US)

(21) Appl. No.: 19/547,335

(22) Filed: Feb. 23, 2026

Related U.S. Application Data
(60) Provisional application No. 63/761,682, filed on Feb. 21, 2025.

(51) Int. Cl. A61K 41/00; C07K 14/78; C07K 16/18; C12N 9/14; C12N 15/62; C12N 15/86

(52) U.S. Cl. CPC A61K 41/0057; C07K 14/78; C07K 16/18; C12N 9/14; C12N 15/62; C12N 15/86; C07K 2317/569; C07K 2319/60; C12Y 308/01005

(57) ABSTRACT

Methods and kits are provided for inducing disassembly of intermediate filaments (IFs) in cells or tissues. In some embodiments, a method comprises expressing in, or introducing into, a cell or tissue a fusion protein comprising a self-labeling protein derived from a haloalkane dehalogenase and an IF-targeting protein that either incorporates into IF polymers or binds specifically to IF proteins; administering a rhodamine- or fluorescein-derived photosensitizer modified with a ligand that specifically binds to the self-labeling protein to associate the photosensitizer with the IF; and administering light under conditions effective to activate the photosensitizer and induce IF disassembly. Kits are also provided that comprise at least one vector encoding a fusion protein comprising a self-labeling protein derived from a haloalkane dehalogenase and an IF-targeting protein, and a rhodamine- or fluorescein-derived photosensitizer modified with a ligand that specifically binds to the self-labeling protein.

### RELATED APPLICATIONS

This application claims priority from U.S. Provisional Application Ser. No. 63/761,682 filed Feb. 21, 2025, the entire disclosure of which is incorporated herein by this reference.

### REFERENCE TO AN ELECTRONIC SEQUENCE LISTING

The contents of the electronic sequence listing (HHMI_25011US_Sequence_Listing.xml; Size: 4,967 bytes; and Date of Creation: Feb. 23, 2026) are herein incorporated by reference in its entirety.

### TECHNICAL FIELD

The presently disclosed subject matter generally relates to tools for inducing intermediate filament (IF) disassembly. In particular, certain embodiments of the presently disclosed subject matter relate to a light-activated tool that makes use of a fusion protein and a rhodamine- or fluorescein-based photosensitizer to induce IF disassembly.

### INTRODUCTION

Intermediate filaments are a family of cytoskeletal polymers that are widely expressed in eukaryotic cells and play important roles in maintaining cellular architecture, mechanical resilience, and intracellular organization. To date, more than seventy IF genes have been identified, each encoding a fibrous protein that either self-assembles or heteropolymerizes with related IF monomers to form mature filaments.

Unlike actin filaments and microtubules, intermediate filaments are apolar, relatively stable polymers that form extended networks spanning the cytoplasm and, in some cases, the nucleus. Distinct subclasses of intermediate filament proteins, including vimentin, desmin, glial fibrillary acidic protein (GFAP), peripherin, keratins, and nuclear lamins, are expressed in a cell type- and tissue-specific manner and have been implicated in processes ranging from development and differentiation to wound healing, fibrosis, neurodegeneration, and cancer progression.

Despite their importance, experimental interrogation of intermediate filament function has historically lagged behind that of other cytoskeletal systems. Actin filaments and microtubules can be rapidly and reversibly perturbed using well-characterized small-molecule agents that directly affect polymerization dynamics, such as latrunculins, cytochalasins, taxanes, and vinca alkaloids. In contrast, intermediate filaments lack comparably specific and fast-acting chemical modulators. As a result, investigators have relied on indirect or coarse perturbation strategies that do not afford precise temporal or spatial control over filament integrity.

Existing approaches for altering intermediate filament organization include genetic knockdown or knockout of filament proteins, expression of dominant-negative mutants, overexpression of tagged fusion proteins, or exposure of cells to broadly acting chemical or physical stressors.

Genetic strategies, while specific at the level of target identity, typically require days to manifest phenotypic effects and often elicit compensatory cellular responses that obscure acute filament-dependent processes. Moreover, genetic depletion eliminates both polymerized filaments and soluble filament subunits, making it difficult to distinguish the functional contributions of different assembly states.

Chemical and physical perturbation methods have also been used to disrupt intermediate filaments, but these approaches suffer from substantial limitations. Strong oxidants, electrophilic compounds, phosphatase inhibitors, aliphatic alcohols, hypotonic challenge, steroidal lactones, and other non-specific treatments have been reported to induce intermediate filament reorganization or breakdown. However, such interventions broadly affect cellular redox balance, protein modification, membrane integrity, and signaling pathways, leading to pleiotropic effects that cannot be readily attributed to selective filament disruption. In many cases, these treatments induce widespread cytotoxicity, organelle damage, or collapse of other cytoskeletal systems, thereby confounding interpretation of observed phenotypes.

Optical methods for manipulating protein function, including chromophore-assisted light inactivation (CALI) and related photochemical techniques, have been developed to achieve spatially confined perturbations in living cells. These approaches typically rely on excitation of a photosensitizing chromophore to generate reactive oxygen species that locally inactivate nearby biomolecules. While such techniques have been applied to individual enzymes, signaling proteins, or organelles, their application to large, polymeric cytoskeletal assemblies has been limited. In particular, existing optical inactivation strategies generally require high light doses, suffer from poor targeting specificity, or result in extensive collateral damage beyond the intended target.

There is an absence of effective tools that allow rapid, selective, and spatially controlled disruption of intermediate filaments in living cells without inducing global cellular damage or relying on slow genetic interventions. This technological gap has limited the ability to probe structure, dynamics, and cellular functions of intermediate filaments.

Accordingly, there remains a need in the art for tools and methods that enable precise, controllable, and selective perturbation of intermediate filament networks in biological systems.

### SUMMARY

The presently disclosed subject matter meets some or all of the above-identified needs, as will become evident to those of ordinary skill in the art after a study of information provided in this document.

This Summary describes several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter relates to methods and kits for inducing disassembly of intermediate filaments (IFs) in cells or tissues in a controlled and selective manner. IFs are a major component of the cytoskeleton and play important roles in cellular architecture, mechanical stability, intracellular organization, and stress responses. Despite their importance, tools for rapidly and selectively perturbing IF networks with spatial and temporal precision have been limited. The disclosed subject matter addresses this need by providing approaches that enable light-activated disruption of IFs under defined conditions.

The presently disclosed subject matter includes methods for inducing disassembly of an intermediate filament in a cell or tissue. The methods include expressing or introducing into the cell or tissue a fusion protein comprising a self-labeling protein derived from a haloalkane dehalogenase and an IF-targeting protein, administering a ligand-modified photosensitizer that specifically binds to the self-labeling protein, and administering light under conditions effective to activate the photosensitizer and induce IF disassembly. By localizing the photosensitizer to intermediate filaments through the fusion protein, light activation results in a localized photochemical effect that disrupts filament integrity without requiring global cellular damage.

The IF-targeting protein component of the fusion protein may incorporate into intermediate filament polymers or may bind specifically to IF proteins, thereby localizing the self-labeling protein, and thus the photosensitizer, to assembled IF networks. The fusion protein may be expressed from a nucleic acid delivered to the cell or tissue or may be introduced as a pre-formed polypeptide. Expression or introduction may occur in cultured cells, tissues, or in vivo in a live organism. The methods are applicable to a wide range of intermediate filament systems and cell or tissue types and may be performed in vitro, ex vivo, or in vivo.

Following localization of the photosensitizer to intermediate filaments, light is administered to activate the photosensitizer. The wavelength, intensity, duration, and spatial distribution of the administered light may be selected based on the photosensitizer used, the biological context, and the desired extent or localization of IF disassembly. Light may be administered globally to induce widespread IF disruption or locally to induce spatially restricted disassembly within a defined subcellular region. In some embodiments, IF disassembly may be achieved without detectable disruption of actin filaments, microtubules, or other cellular structures, and without inducing global cellular phototoxicity.

The disclosed methods provide a powerful research platform for interrogating the immediate roles of intermediate filaments in cellular physiology and pathology. By enabling rapid, selective, and spatially controlled IF disassembly in living cells, the methods allow observation of acute cellular responses to IF loss, including changes in cell shape, mechanical behavior, cytoskeletal coordination, and organelle positioning. Such acute effects are difficult or impossible to capture using genetic knockouts or chronic perturbation approaches, which may introduce compensatory changes over time.

The disclosed subject matter also provides a bridge between basic cell biology and translational research. The ability to induce IF disassembly with temporal and spatial precision enables targeted investigation of redox-sensitive cytoskeletal perturbations, mechanical stress responses, and cytoskeletal cross-talk under conditions that more closely resemble acute pathological stress. As such, the disclosed methods may be used to refine disease models, uncover mechanistic vulnerabilities in diseased cells, and support hypothesis testing and preclinical discovery in contexts where intermediate filaments are implicated, including but not limited to cancer progression, neurodegenerative and neuroinflammatory disorders, muscle and cardiac diseases, and epithelial stress pathologies.

The presently disclosed subject matter further includes kits for inducing disassembly of intermediate filaments. The kits comprise at least one vector encoding a fusion protein comprising a self-labeling protein and an IF-targeting protein, and a photosensitizer modified with a ligand that specifically binds to the self-labeling protein. The kits may optionally include additional photosensitizers, carriers, buffers, or other components suitable for storage, handling, or use. By providing both the genetic and chemical components required for targeted photosensitizer localization, the kits facilitate flexible implementation of IF disassembly across a range of experimental and biological contexts.

The disclosed methods and kits thus provide a modular and controllable approach for inducing intermediate filament disassembly with spatial and temporal precision, enabling new experimental capabilities for studying IF function and dynamics in living systems.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A-1E: Vimentin-HaloTag® protein incorporates into the endogenous Vimentin intermediate filament (VIF) network.

FIG. 1A: Airyscan images of a chemically fixed African green monkey kidney fibroblast-like cells (COS-7) expressing Vimentin-HaloTag® protein-JFX₆₅₀ (left) and immunostained for vimentin (right) FIG. 1B: Pixel-wise intensity distribution for the images shown in FIG. 1A with Pearson's Correlation Coefficient (PCC). FIG. 1C: Projected, denoised tomogram of a single vimentin filament in a mouse embryonic fibroblasts (MEF). Densities corresponding to HaloTag® protein are shaded. FIG. 1D: Frequency distribution of HaloTag® protein to VIF distances along 32 filaments from 5 tomograms. FIG. 1E: Three-dimensional rendering indicating position of vimentin-HaloTag® protein incorporation into a single VIF. Rendering is annotated with mean HaloTag® protein copy number per 100 nm and mean HaloTag® protein distance from filament computed from the full dataset.

FIG. 2A-2F: Acute fragmentation of vimentin IFs upon excitation of a vimentin-targeted photosensitizer. FIG. 2A: Schematic outlining the FilaBuster implementation. Step 1: Vimentin-HaloTag® protein is expressed and co-assembles with native filaments. Step 2: Cells are labeled with JF₅₇₀-HTL, a cell-permeable photosensitizer that covalently binds HaloTag. Step 3:561 nm light excites the photosensitizer dye, generating reactive oxygen species (ROS) to drive localized filament disassembly. FIG. 2B: Airyscan images showing colocalization of stably expressed vimentin-HaloTag® protein (JFX₅₄₉, left) with endogenously tagged vimentin-mEmerald (right) in COS-7 cells. FIG. 2C: Tomogram slice of a VIF from a vimentin-HaloTag® protein-expressing MEF, with HaloTag® protein densities indicated by arrows. FIG. 2D: 3D model of a VIF with associated HaloTag® proteins (spheres) reconstructed from cryo-ET. FIG. 2E: Airyscan montage of a COS-7 cell expressing vimentin-HaloTag® protein labeled with JF₅₇₀-HTL. 561 nm illumination induces progressive VIF disassembly over ~4 minutes.

FIG. 2F: Airyscan montage of endogenously tagged vimentin-mEmerald (top row) and co-expressed vimentin-HaloTag® protein-JF₅₇₀ (bottom row) in a COS-7 cell (images in f are gamma adjusted (0.5) for improved visualization of filaments). 561 nm laser power (measured at the sample plane), irradiance, pixel dwell time, and energy dose per pixel are indicated for panels FIG. 2E and FIG. 2F.

FIG. 3A-3D: Dynamics of FilaBuster-mediated IF network disintegration. FIG. 3A: Widefield montage of VIF disassembly in a MEF labeled with vimentin-HaloTag® protein-JF₅₇₀. FIG. 3B: Airyscan timelapse of VIF fragmentation in a COS-7 cell expressing vimentin-HaloTag® protein-JF₅₇₀. VIF disassembly results in a more homogeneous fluorescence signal throughout the image reflected in the decreasing coefficient of variation over time. FIG. 3C: Additional timelapse showing VIF disassembly in a vimentin-HaloTag® protein-JF₅₇₀ labeled COS-7 cell. The mean normalized radial distribution of vimentin intensity between the nuclear envelope (0.0), and the plasma membrane (1.0) is plotted over time. Loss of filament integrity is associated with a more uniform distribution of VIF fragments throughout the cytoplasm. FIG. 3D: Widefield timelapse of VIF fragmentation in a vimentin-HaloTag® protein-JF₅₇₀ labeled MEF. Light-mediated VIF fragmentation results in the disappearance of dense filament bundles and a steep drop in local orientational coherency within the image. 561 nm laser power, irradiance, pixel dwell time, and energy dose per pixel are reported for confocal experiments FIG. 3A and FIG. 3B. Laser power, irradiance, exposure time, energy dose per frame, and integrated energy dose to VIF fragmentation are reported for FIG. 3C.

FIG. 4A-4D: VIF disassembly by activation of a nanobody-targeted photosensitizer. FIG. 4A: Cartoon schematic of the FilaBuster strategy using a Vimentin-V_{H}H/nanobody-HaloTag® protein targeting strategy. FIG. 4B-4C: Airyscan images of vimentin-V_{H}H-HaloTag® protein-JF₅₇₀ (top) and endogenously tagged vimentin (mEmerald, bottom) in COS-7 cells. After 100 frames/illumination cycles, the vimentin-associated nanobody signal disappears and the endogenous vimentin appears highly fragmented. FIG. 4D: Airyscan montage of vimentin-V_{H}H-HaloTag® protein-JF₅₇₀ (top) and vimentin-mEmerald (knock-in, bottom) in a COS-7 cell. Five minutes into the movie (frame 55), the 561 nm laser power is raised two orders of magnitude, activating the photosensitizer and driving vimentin IF disassembly.

FIG. 5A-5F: A novel far-red emitting photosensitizer HaloTag® ligand. FIG. 5A: Characterization of JF₆₃₄ and JF₆₃₄-HTL. FIG. 5B: Excitation/emission spectra for JF₆₃₄-HTL. FIG. 5C: JF₆₃₄-HTL is fluorogenic, displaying ~300× greater absorbance upon HaloTag® ligand binding. FIG. 5D: Cartoon schematic indicating adapted FilaBuster strategy with red-shifted HaloTag® ligand and longer wavelength excitation light. FIG. 5E: Airyscan montage of a COS-7 cell expressing vimentin-HaloTag® protein-JF₆₃₄ illuminated with 639 nm light (power: 2.8 μW; energy dose: 8.1×10⁻³ J/cm²). FIG. 5F: Widefield epifluorescence timelapse of a stable vimentin-HaloTag® protein MEF labeled with JF₆₃₄-HTL.

FIG. 6A-6E: Expanding the FilaBuster strategy with conventional HaloTag® ligands. FIG. 6A: Schematic indicating the effect of light-dose on conventional HTLs targeted to VIFs. In low-power “standard imaging mode,” vimentin-HaloTag® protein labeled with a conventional HTL can be imaged for extended periods without disrupting filament integrity. In “FilaBuster mode,” enhanced illumination results in VIF fragmentation. FIG. 6B: Cartoon depicting various conventional, non-photosensitizer ligands that can be used to label VIF-targeted HaloTag. FIG. 6C: Widefield epifluorescence timelapse of a MEF expressing vimentin-HaloTag® protein-JF₆₅₇ acquired in standard imaging mode. Over 601 illumination cycles with low 640 nm laser power (0.052 mW) no detectable VIF fragmentation were observed. FIG. 6D: Timelapse of a MEF expressing Vimentin-HaloTag® protein-JF₆₅₇ acquired in “FilaBuster mode.” Illuminating with ~50× higher 640 nm laser power, clear VIF fragmentation was observed within ~150 cycles. FIG. 6E: Time (sec) to VIF fragmentation in vimentin-HaloTag® protein MEFs labeled with the indicated HaloTag® ligand. Cells were excited at 488 nm, 561 nm, or 640 nm at 1 frame per second, 100 ms exposure, and the indicated irradiance. Time to VIF fragmentation in seconds corresponds to number of illumination cycles. JF₆₀₈ was screened with both 561 nm and 640 nm excitation. Boxplots show median (center line), interquartile range (box), and data points within 1.5× the IQR (whiskers); outliers are plotted individually and cells with no visible VIF fragmentation at 10 minutes are plotted at 600 seconds (max value). For panels FIG. 6C and FIG. 6D, 640 nm laser power (measured at the sample plane), irradiance, exposure time, energy dose per frame, and total energy dose are indicated. Summary table of illumination parameters, performance metrics, and sample size for each condition is included in FIG. 7B.

FIG. 7A-7C: Benchmarking JF-HTL FilaBuster efficiency in vimentin-HaloTag® protein MEFs. FIG. 7A: Structures of HaloTag® ligands tested, including conventional rhodamine-based dyes and photosensitizer dyes. FIG. 7B: Summary table of illumination parameters and performance metrics for each ligand in vimentin-HaloTag® protein MEFs using widefield epifluorescence microscopy. All experiments were performed using a 63×/1.4 NA oil objective, 100 ms exposure time, and 1 frame per second acquisition. Shown are laser wavelength used and power measured at the sample plane, irradiance, energy dose per frame, average time to VIF fragmentation (equivalent to number of illumination cycles), and average total energy dose to fragmentation. FIG. 7C: Integrated energy required for VIF disassembly plotted against ligand absorbance at the laser line used for excitation. Bubble size reflects the variability in fragmentation latency. This data set serves as a practical reference for implementing FilaBuster across different ligands and imaging platforms. Note: JF₆₀₈ was screened with both 561 nm and 640 nm laser lines.

FIG. 8A-8E: FilaBuster-mediated VIF disassembly is reproducible across cell types. FIG. 8A: retinal pigment epithelial cells (RPE1) expressing vimentin-HaloTag® protein-JFX₅₄₉ before (left) and after (right) 561 nm irradiation. FIG. 8B: Potorous tridactylus kidney epithelial cells (PTK2) expressing vimentin-HaloTag® protein-JFX₅₅₄ before (left) and after (right) 561 nm irradiation. FIG. 8C: human embryonic kidney 293T cell (HEK293T) expressing vimentin-HaloTag®protein-JFX₅₄₉ before (left) and after (right) 561 nm irradiation. FIG. 8D. Mouse embryonic fibroblasts (NIH/3T3) expressing vimentin-HaloTag® protein-JF₅₅₂ before (left) and after (right) 561 nm irradiation. FIG. 8E. Normal human epidermal fibroblast expressing vimentin-HaloTag® protein-JF₅₅₂ before (left) and after (right) 561 nm irradiation. FIG. 8F. Endogenously tagged vimentin-HaloTag® protein in a human osteosarcoma cell (U2-OS) before (left) and after (right) 642 nm irradiation.

FIG. 9A-9D: Defining FilaBuster operational boundaries and failure modes. FIG. 9A: Cartoon schematic indicating the two boundary conditions that were investigated: expression of a non-directed HaloTag® protein and expression of an F-actin-directed HaloTag® protein. FIG. 9B: Airyscan timelapse of endogenously tagged vimentin (vimentin-mEmerald) and unfused HaloTag® protein-JFX₅₄₉ in a COS-7 cell. At frame 125, the left cell is extensively photobleached with 561 nm light, but no effect on VIF stability was observed seven minutes later. FIG. 9C: Testing target specificity. Airyscan montage of lifeact-HaloTag® protein-JF₆₃₄ and vimentin-mEmerald in a COS-7 cell. Prolonged irradiation of the f-actin-targeted photosensitizer does not result in global actin filament fragmentation or loss of VIF integrity. FIG. 9D: Table comparing FilaBuster (current work) with relevant publications using various configurations of chromophore assisted light inactivation (CALI).

FIG. 10A-10B: SuperNova2 and SNAP-tag fusions do not support efficient light-mediated VIF disassembly. FIG. 10A: Testing the compatibility of a genetically encoded photosensitizer fluorescent protein. Airyscan montage of vimentin-SuperNova2 (top) and endogenously tagged vimentin (mEmerald, bottom) in a COS-7. After ~60 cycles of 561 nm illumination, the SuperNova2 tag fully photobleaches but no VIF destabilization was observed.

FIG. 10B: Testing the compatibility of SNAP-tag, an alternative self-labeling protein. Airyscan timelapse of a MEF stably expressing vimentin-SnapTag-JFX₅₅₄ displayed negligible VIF fragmentation over 896 cycles of 561 nm illumination.

FIG. 11A-11I: Vimentin C328 is dispensable for FilaBuster-mediated VIF fragmentation. FIG. 11A: Airyscan micrographs of stable COS-7 cells expressing vimentin-C328H-HaloTag® protein-JF₆₅₇ before (left) and after (right) VIF disintegration. FIG. 11B: Enlarged inset from A showing montage of VIF disassembly. FIG. 11C-11D. Coefficient of variation (FIG. 11C) and average local coherency (FIG. 11D) indicating time-course of VIF disintegration over the full movie shown in FIG. 11B. FIG. 11E: Airyscan micrographs of a COS-7 cell stably expressing vimentin-C328A-HaloTag® protein-JF₆₃₄ before (left) and after (right) VIF fragmentation. FIG. 11F-11H: Vimentin knockout MEFs rescued with either (FIG. 11F) Vimentin-HaloTag® protein-JFX₅₄₉, (FIG. 11G) vimentin-C328H-HaloTag® protein-JFX₅₅₄, or (FIG. 11H) vimentin-C328A-HaloTag® protein-JFX₅₄₉. Expression of 100% tagged vimentin produces truncated ~3 μm filaments which undergo clear disintegration in both control and C328 mutants, confirming that FilaBuster-mediated VIF fragmentation does not require cysteine. FIG. 11I: Vimentin knockout MEF rescued with the conventional vimentin-mNeonGreen fusion. Expression of 100% tagged vimentin-mNeonGreen results in comparable truncated filaments, which do not undergo fragmentation.

FIG. 12A-12B: FilaBuster drives vimentin methionine oxidation but minimal global oxidative stress. FIG. 12A: Table indicating differential vimentin methionine oxidation in vimentin-HaloTag® protein knock-in U2-OS cells under three conditions. In condition A, cells were labeled with Biotin-JF₆₄₆-HaloTag® ligand and irradiated with 639 nm light to drive VIF fragmentation. In condition B, cells were dye labeled but were not irradiated. In condition C, cells were neither labeled nor irradiated. FIG. 12B: Airyscan montage of a vimentin knock-in (mEmerald, white) COS-7 expressing Vimentin-HaloTag® protein-JFX₅₄₉ (not shown). The cell was labeled with CellRox DeepRed to monitor changes to global cellular ROS levels. This probe primarily localizes to mitochondria, and no detectable change in its intensity were observed in the 1.5 hours after VIF disassembly.

FIG. 13A-13E: FilaBuster enables precise spatial control over VIF fragmentation. FIG. 13A: Cartoon schematic indicating focal irradiation strategy for targeted VIF disassembly. FIG. 13B: Airyscan montage of a COS-7 stably expressing Vimentin-HaloTag® protein-JFX₆₅₀. The cell was focally irradiated with a 639 nm laser at the indicated region (box), resulting in localized VIF disassembly. FIG. 13C: Focal irradiation of a dense, perinuclear vimentin cap. Within 30 seconds of 639 nm irradiation, VIFs within the irradiation window (box) fragment and the cap is bisected. Over several minutes, the two hemispheres of the cap continue to separate, and a subtle increase in soluble vimentin background was observe in the cytoplasm. FIG. 13D: Airyscan montage of a dense vimentin IF bundle labeled with vimentin-HaloTag® protein-JFX₆₅₀. Brief 639 nm irradiation at the indicated region (box) resulted in targeted IF disassembly, effectively bisecting the bundle within 2 minutes. FIG. 13E: Airyscan montage of an extended vimentin IF bundle labeled with vimentin-V_{H}H-HaloTag® protein-JFX₅₄₉. Focal 561 nm irradiation (box) severs the bundle, resulting in retraction of the two free ends (arrows).

FIG. 14A-14D: Focal irradiation of conventional vimentin-FP fusions does not induce VIF fragmentation. FIG. 14A: Airyscan timelapse of vimentin-mStayGold. At frame 61, StayGold is photobleached at the indicated region. Over the subsequent 15 minutes, there is extensive recovery due to trafficking of new filaments into the bleach site and no evidence of VIF fragmentation. FIG. 14B-14C: MEFs co-expressing vimentin-HaloTag® protein-JFX₆₄₆ with the conventional reporter vimentin-mNeonGreen. FIG. 14B: After photobleaching mNeonGreen with 488 nm light, there is no evidence of VIF fragmentation. FIG. 14C: After photobleaching JFX₆₄₆ with 639 nm light, robust VIF fragmentation was observed, visible in the mNeonGreen channel as well (arrows). FIG. 14D: Airyscan montage of a U2-OS co-expressing the conventional reporter vimentin-mEmerald along with vimentin-V_{H}H-HaloTag® protein-JFX₅₄₉ (not shown). 561 nm irradiation of the JFX₅₄₉-labeled nanobody at the indicated region (box), results in localized vimentin IF disassembly visible with the conventional mEmerald reporter.

FIG. 15A-15G: Kinetics of VIF fragmentation. FIG. 15A: TIRF montage of VIF fragmentation in a U2-OS cell expressing vimentin-HaloTag® protein-JFX₆₄₆. Arrows indicate sites of filament scission. FIG. 15B: Airyscan timelapse capturing fragmentation of a single VIF labeled with vimentin-HaloTag® protein-JF₅₅₂ in a COS-7 cell. FIG. 15C: Comparison of VIFs before (left) or immediately after (right) fragmentation. FIG. 15D: MSD of VIF fragments computed from single particle tracking of VIF fragments either in the first 10 seconds after VIF disassembly, or 30-40 seconds after VIF disassembly. In each case, alpha values were fit over short (<45 msec) and longer (100-600 msec) lag times. FIG. 15E: Cartoon schematic indicating VIF fragmentation and increase in soluble VIF subunits using the FilaBuster approach. FIG. 15F: Global VIF fragmentation is accompanied by a rapid increase in diffuse vimentin-HaloTag® protein signal in regions devoid of filaments prior to irradiation, consistent with increased soluble VIF subunits. FIG. 15G. Average fluorescence recovery after photobleaching (mean±s.d.) of confined VIF fragments (n=5) display modest recovery over 4 minutes, consistent with continued subunit exchange.

FIG. 16A-16C: VIF fragments coalesce and inhibit new filament assembly. FIG. 16A: Airyscan montage of vimentin-HaloTag® protein-JF₆₀₈ in a COS-7 at the indicated times post-VIF disassembly (hh:mm). Scale bar is 5 μm. Line indicates line scan used for FIG. 16B. FIG. 16B: Kymograph displaying retrograde motion of aggregated VIF fragments over 3+ hours with average retrograde speed of ~25 nm/min. FIG. 16C: Pulse chase labeling of vimentin-HaloTag® protein 20 hours after 561 nm light-mediated VIF fragmentation. COS-7 cells were initially labeled with JF₅₄₉-HTL, extensively washed, and irradiated to drive VIF disintegration. Twenty hours post-irradiation, cells were labeled with JF₆₃₅-HTL (chase dye) for 1 hour to label any newly translated vimentin-HaloTag® protein after VIF fragmentation.

FIG. 17A-17E: Radial expansion of VIF fragments and perinuclear organelles upon light-induced VIF disassembly. FIG. 17A: Rapid light-mediated disassembly of a collapsed perinuclear VIF network in a nocodazole-treated COS-7 cell. VIF fragments expand outward over 15-20 minutes. FIG. 17B: Bounding area of perinuclear VIF density after 639 nm irradiation indicating radial expansion of VIF fragments. FIG. 17C: (left) A radially expanding wave of VIF fragments upon 633 nm irradiation of vimentin-HaloTag® protein-JF₆₃₅ in a taxol-treated COS-7 cell. (right) Kymograph of radial vimentin-HaloTag® protein-JF₆₃₅ intensity over time. Radial intensity distribution at each time point represents the median of 360 radial line scans from the center of the cell, in 1° increments. VIF disintegration at ~10 min, is accompanied by the rapid expansion of VIF fragments traveling at ~497 nm/min. FIG. 17D-17E: Trajectories of perinuclear, VIF-associated lipid droplets either before (FIG. 17D) or after (FIG. 17E) VIF fragmentation.

FIG. 18A-18H: Cellular reorganization upon acute VIF fragmentation. FIG. 18A: Airyscan montage of a COS7 cell expressing Vimentin-HaloTag® protein-JF₅₇₀. Cellular reorganization is visible within minutes of initial VIF fragmentation. FIG. 18B: Time projection of the cell boundary from the cell indicated in a. FIG. 18C: Airyscan images of a COS-7 cell expressing vimentin-HaloTag® protein-JF₆₀₈ and a microtubule marker (EMTB-3×GFP). Global IF disintegration is apparent at 7 minutes, with only subtle changes to the organization and density of microtubules. FIG. 18D: Correlated fluorescence (left) and transmitted light (right) images of a COS-7 expressing vimentin-HaloTag® protein-JF₆₃₄ before and after VIF disintegration. White lines indicate the border of the perinuclear organellar mass. FIG. 18E: Mean velocity vectors from PIV analysis indicating motion of perinuclear organelle mass upon VIF disassembly. FIG. 18F: Widefield montage of a COS-7 expressing vimentin-HaloTag® protein-JFX₆₅₀ (top) and labeled with Bodipy 493/503 to visualize lipid droplets (LD, bottom). At t=3 min, the 642 nm laser power was increased, driving global VIF fragmentation and concomitant LD radial motion. FIG. 18G: Position of a representative LD over the entire movie, with arrows indicating moment of VIF disassembly. FIG. 18H: MSD of LD trajectories either before or after light-mediated VIF fragmentation. Alpha values for each MSD plot are included for reference.

FIG. 19A-19D: FilaBuster is a generalizable approach for IF fragmentation. FIG. 19A: Airyscan images of a COS-7 expressing GFAP-HaloTag® protein-JF₅₅₂ before and after 561 nm light-induced GFAP filament disintegration. FIG. 19B-19C: Airyscan images of U2-OS cells expressing Desmin-HaloTag® protein-JFX₅₄₉ (FIG. b) or Peripherin-HaloTag® protein-JFX₅₄₉ (FIG. 19C) before and after light-induced filament disintegration. FIG. 19D: Airyscan images of a COS-7 expressing Keratin18-HaloTag® protein-JF₅₅₂ before and after light-mediated disassembly.

FIG. 20A-20G: Vimentin FilaBuster does not induce global oxidative stress or phototoxicity at relevant light doses. FIG. 20A-20C: Dose-response relationship between 561-nm illumination, VIF fragmentation, and cellular oxidative stress. Cells labeled with JF₅₇₀-HTL (500 nM, vimentin) and CellROX Green (2.5 μM, ROS indicator) were subjected to widefield epifluorescence using three illumination regimes (parameters and cumulative doses indicated). VIF fragmentation quantified as normalized coefficient of variation (CoV), ROS measured as CellROX intensity change. Arrows indicate onset of VIF fragmentation. At low light dose, VIF fragmentation begins after ~150 illumination cycles without detectable ROS generation. Higher light doses fragment VIFs more rapidly but continued illumination triggers increased oxidative stress. FIG. 20D: Endpoint CellROX response across illumination regimes, showing individual cells (points) and distribution summary (box plot). FIG. 20E: ER calcium release is not triggered by FilaBuster. Representative cell coexpressing ER-anchored, cytosol-facing ER-GCaMP6f and labeled for vimentin (HaloTag-JF₅₄₉). Images show vimentin and ER-GCaMP6f before VIF fragmentation, after global VIF fragmentation induced by 561-nm illumination, and after a positive-control photodamage stimulus (405-nm, 5-s spot irradiation), which produces a robust increase in ER-GCaMP6f signal consistent with ER calcium leak into the cytoplasm. Scale bar, 20 μm. FIG. 20F: Time traces from two representative cells showing VIF fragmentation (top, normalized CoV) and cytosolic calcium (bottom, ER-GCaMP6f AF/F) during FilaBuster (561 nm) and subsequent photodamage (405 nm). Vertical dashed lines mark illumination timing. FIG. 20G: Mitochondrial depolarization is not triggered by FilaBuster. Cells labeled with vimentin-HaloTag® protein-JF₆₄₆ and TMRM (75 nM, mitochondrial membrane potential) show maintained TMRM signal after VIF fragmentation but loss after photodamage (405-nm). Scale bar, 25 μm.

FIG. 21A-21F: Intermediate-filament fragmentation does not grossly perturb microtubules, mitochondria, or lysosome motility. FIG. 21A-21C: Microtubules remain intact during vimentin IF fragmentation. Vimentin-HaloTag® protein knock-in COS-7 cells were labeled with HaloTag® ligand (JF₅₄₉ or JF₆₄₆, as indicated) to visualize vimentin and with SPY-tubulin to label endogenous microtubules. FIG. 21A: Global 561-nm illumination (FilaBuster) induces widespread vimentin IF fragmentation without a corresponding loss of microtubule organization. FIG. 21B: Focal FilaBuster activation within the boxed region induces local vimentin fragmentation while the microtubule network within and outside the irradiated region remains continuous. FIG. 21C: Time-lapse montage of the boxed region shows progressive loss of vimentin filament continuity with preserved microtubule morphology over the same interval (display gamma as indicated). Scale bars as indicated. FIG. 21D: Mitochondria are not detectably damaged by focal keratin IF disassembly. HeLa cells coexpressing mito-dsRed2 (mitochondrial matrix) and Keratin18-HaloTag® protein labeled with JF₆₄₆ were subjected to focal 639-nm irradiation to induce local keratin filament disassembly (dashed boundary). Mitochondria within the irradiated/keratin-disassembled region do not show obvious morphological abnormalities relative to adjacent non-irradiated regions. Insets show a higher-magnification view of the boundary. FIG. 21E-21F: Global lysosome motility is not grossly altered by vimentin IF fragmentation. Vimentin-HaloTag® protein knock-in COS-7 cells were labeled with JF₅₄₉ and LysoTracker Deep Red to label lysosomes. FIG. 21E: (e) Representative cell imaged before (top) and after (bottom) global FilaBuster-induced vimentin fragmentation. Lysosome trajectories from 400 sequential frames are visualized as cumulative maximum-intensity projections, shown over progressively increasing time windows to reveal trajectory paths. FIG. 21F: Automated lysosome tracking (TrackMate) summary: mean speed per trajectory plotted against trajectory stop time within the movie for all trajectories pooled across cells (3,400 trajectories). No gross reduction in lysosome speeds is observed after vimentin fragmentation on this global measure.

FIG. 22A-22C: Cells remain viable after global FilaBuster and re-establish the endogenous vimentin IF network over ~20 hours. FIG. 22A-22B: Field-of-view examples before FilaBuster and 8 h after global VIF fragmentation. Prior to irradiation, vimentin is organized as an extended filamentous network with perinuclear enrichment. Following FilaBuster, vimentin signal redistributes into diffuse, punctate fragments that are broadly dispersed throughout the cytoplasm. Cells maintain spread morphology and remain attached/viable over this interval, with no obvious increase in cell loss. FIG. 22C: Long-term time course showing VIF fragmentation followed by gradual recovery of filamentous organization. Immediately after global FilaBuster (0 h), vimentin fluorescence is largely non-filamentous and dispersed. Over the ensuing hours, vimentin progressively reorganizes from dispersed fragments into a re-emerging perinuclear network and then into an extended filamentous IF array by ~20 h. Arrowheads mark cells undergoing division during the recovery period, indicating that cells not only survive acute IF fragmentation but can progress through mitosis and re-establish the endogenous vimentin network.

FIG. 23A-23D: Target specificity controls for HaloTag® protein/ligand-anchored photosensitizer activation. FIG. 23A: HaloTag® protein on vimentin IFs (FilaBuster condition). Vimentin-HaloTag® protein labeling shows rapid, progressive IF fragmentation under 561-nm illumination, transitioning from intact filaments to a fully fragmented network. FIG. 23B: HaloTag® protein on microtubules (β-tubulin-HaloTag® protein). Under identical illumination, microtubules remain largely intact over the interval, with only partial microtubule fragmentation evident at later time points. FIG. 23C: HaloTag® protein on actin filaments (LifeAct-HaloTag® protein). Identical illumination produces a gradual loss of F-actin signal/structures, consistent with actin network disruption (arrowheads indicate representative regions of loss). FIG. 23D: HaloTag® protein in mitochondria (matrix-targeted HaloTag® protein). 561-nm illumination of mitochondrially targeted photosensitizer induces mitochondrial damage, progressing from normal morphology to swelling and fragmentation (arrowheads mark early damage).

FIG. 24A-24C: Photochemical disassembly of multiple cytoplasmic IF polymers in the absence of vimentin. FIG. 24A: GFAP IF FilaBuster in vimentin-KO MEFs. Vim-KO MEFs expressing GFAP-HaloTag® protein labeled with JF₅₄₉ were imaged before and after global 561-nm activation. GFAP forms a filamentous network in the absence of vimentin and undergoes rapid conversion from intact filaments to punctate fragments following FilaBuster. Lower panels show enlarged views of the boxed region, highlighting fragmentation of GFAP filaments. FIG. 24B: Desmin IF FilaBuster in vimentin-KO MEFs. Vim-KO MEFs expressing desmin-HaloTag® protein labeled with JF₅₄₉ show a filamentous desmin network prior to activation and a fragmented, punctate distribution after global FilaBuster. Insets (bottom) show magnified views of the boxed region. FIG. 24C: Keratin filament fragmentation in mitosis. HeLa cells expressing keratin18-HaloTag® protein labeled with JF₅₄₉ were imaged during metaphase. Global FilaBuster drives rapid loss of cortical keratin filaments and accumulation of bright keratin condensates over minutes. Fragmentation is accompanied by an apparent decrease in cortical integrity/rounding consistent with reduced cortical tension in this example.

### DESCRIPTION OF EXEMPLARY EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

### Methods for Inducing Disassembly of Intermediate Filaments

The presently disclosed subject matter includes methods for inducing disassembly of an intermediate filament (IF) in a cell or tissue. The methods are applicable to cells or tissues in vitro, ex vivo, or in vivo, and may be performed in cultured cells, tissues, or in a live organism. The methods provide controlled, light-activated disruption of intermediate filament networks by localizing a photosensitizing compound to the IF and activating the compound with light under conditions effective to induce filament disassembly.

Expression or Introduction of an IF-Targeted Fusion Protein. In accordance with the methods disclosed herein, a fusion protein is expressed in, or introduced into, the cell or tissue. The fusion protein comprises a self-labeling protein derived from a haloalkane dehalogenase and an IF-targeting protein. The IF-targeting protein may be any protein or protein domain that incorporates into intermediate filament polymers or binds specifically to intermediate filament proteins, thereby localizing the fusion protein to the IF network. By virtue of this targeting, the self-labeling protein is positioned in close proximity to assembled intermediate filaments within the cell or tissue.

The fusion protein may be expressed from a nucleic acid introduced into the cell or tissue using methods known in the art, or may be introduced directly as a pre-formed polypeptide. Expression or introduction may occur in a variety of cellular contexts and does not require modification of all IF subunits within the cell. In some embodiments, the fusion protein co-assembles with endogenous IFs, while in other embodiments the fusion protein binds to IFs through a specific binding interaction without incorporation into the filament polymer.

Administration of a Ligand-Modified Photosensitizer. Following expression or introduction of the fusion protein, a photosensitizer is administered to the cell or tissue. The photosensitizer is a rhodamine- or fluorescein-derived compound that is modified with a ligand that specifically binds to the self-labeling protein. Upon administration, the ligand binds to the self-labeling protein, thereby covalently associating the photosensitizer with the fusion protein and localizing the photosensitizer to the intermediate filament network via the IF-targeting protein.

Light Activation and Induction of IF Disassembly. After the photosensitizer has been associated with the fusion protein, light is administered to the cell or tissue under conditions effective to activate the photosensitizer and induce disassembly of the IF. The wavelength of the administered light is selected to excite the photosensitizer, and the intensity, duration, and spatial extent of illumination may be adjusted based on the photosensitizer used, the cellular context, and the desired extent of IF disassembly.

Upon light activation, the photosensitizer generates a localized photochemical effect at or near the intermediate filament, resulting in disruption of filament integrity and disassembly of the IF network. Light may be administered globally or to a localized region, enabling spatially restricted IF disassembly if desired. Unless otherwise indicated, the method does not require induction of global cellular damage or phototoxicity, and IF disassembly may be achieved without detectable disruption of other cytoskeletal systems or organelles.

The method described herein is applicable to a wide range of intermediate filament systems and cell or tissue types. By combining IF-targeted localization of a self-labeling protein with ligand-directed photosensitizer binding and light activation, the method provides a modular and controllable approach for inducing IF disassembly with temporal and spatial precision. The steps of the method may be varied or combined as appropriate, provided that the photosensitizer is localized to the IF and activated by light under conditions sufficient to induce disassembly.

### Kits for Inducing Disassembly of Intermediate Filaments

The presently disclosed subject matter further includes kits for use in inducing disassembly of IFs in cells or tissues. The kits comprise components that, when used together, enable localization of a photosensitizer to IFs via a self-labeling protein and subsequent light-activated disruption of filament integrity. The kits may be provided as packaged combinations of components and may be configured for use in vitro, ex vivo, or in vivo.

In accordance with the kit, at least one vector is provided that comprises a nucleotide encoding a fusion protein. The fusion protein comprises a self-labeling protein derived from a haloalkane dehalogenase and an intermediate filament (IF)-targeting protein. The vector may include regulatory elements suitable for expression of the fusion protein in a target cell or tissue. The kit does not require that the vector be of a particular type, provided that it is capable of delivering and/or expressing the nucleotide encoding the fusion protein. In some embodiments, the vector is a plasmid vector. In other embodiments, the vector is a viral vector. Exemplary viral vectors include, but are not limited to, lentiviral vectors, adenoviral vectors, and adeno-associated viral vectors. Single-vector systems and multi-vector systems are contemplated, and the kit may include one or more vectors encoding the same or different fusion proteins.

The kit further comprises a photosensitizer that is modified with a ligand that specifically binds to the self-labeling protein. Modification of the photosensitizer with the ligand enables covalent association of the photosensitizer with the self-labeling protein encoded by the vector, thereby localizing the photosensitizer to intermediate filaments via the IF-targeting protein. The photosensitizer may be provided in a form suitable for labeling the fusion protein following expression in a cell or tissue. The kit does not require that the photosensitizer be active in the absence of light, and the photosensitizer may function as a conventional fluorescent label under non-activating conditions. In some embodiments, the kit may further comprise one or more additional photosensitizers having different excitation or emission properties, although such additional photosensitizers are not required.

In some embodiments, the kit further comprises a pharmaceutically acceptable carrier or buffer. Such carriers or buffers may be suitable for storage, handling, or delivery of the photosensitizer, the vector, or both. The carrier or buffer may be selected based on the intended application and may be compatible with cell culture, tissue application, or in vivo use.

The kit may optionally include additional components such as packaging materials, labeling, or instructions for use. Unless otherwise indicated, inclusion of such components is optional, and the kit is not limited to a particular format or presentation, provided that it comprises at least one vector encoding the fusion protein and a ligand-modified photosensitizer capable of binding the self-labeling protein.

The components of the kit may be selected and combined to support localization of a photosensitizer to intermediate filaments and light-activated induction of filament disassembly. The kit is compatible with different IF-targeting strategies, photosensitizer chemistries, and illumination configurations, and may be used in a wide range of cellular and tissue contexts. By providing both the genetic and chemical components required for targeted photosensitizer localization, the kit enables flexible implementation of intermediate filament disassembly without requiring users to independently assemble or source the constituent elements.

### IF-Targeting Proteins

In some embodiments, the IF-targeting protein is an intermediate filament (IF) protein itself. In such embodiments, the fusion protein, comprising the IF protein and the self-labeling protein, incorporates into intermediate filament polymers and co-assembles with endogenous IF subunits. This incorporation localizes the self-labeling protein, and thus the ligand-bound photosensitizer, in close proximity to assembled intermediate filaments, enabling light-activated induction of intermediate filament disassembly. In such embodiments, when the IF protein incorporates into the IF polymers within the cell or tissue, the self-labeling protein is positioned in close proximity to assembled IFs. Incorporation of the fusion protein into IF polymers may occur through co-assembly with endogenous IF subunits during filament formation, subunit exchange, or network remodeling.

The IF protein used as the IF-targeting protein may be any IF protein that forms or participates in cytoplasmic intermediate filament networks. Exemplary IF proteins include vimentin, desmin, glial fibrillary acidic protein (GFAP), peripherin, keratin 18, and nestin. The IF protein may be selected based on the cell type, tissue type, or intermediate filament system of interest. In embodiments where the IF protein is fused to the self-labeling protein, the fusion protein may assemble into homopolymeric or heteropolymeric IF networks and may coexist with endogenous IF proteins without substantially altering baseline filament organization prior to light activation.

In embodiments employing IF protein-based targeting, localization of the self-labeling protein along the length of the filament places the ligand-bound photosensitizer in repeated, distributed proximity to the IF polymer. Upon administration of light, this spatial arrangement facilitates efficient induction of IF disassembly, including fragmentation or loss of filament continuity, without requiring global disruption of cellular structures.

In other embodiments of the presently disclosed subject matter, the IF-targeting protein comprised by the fusion protein is an IF-binding protein that binds specifically to IF proteins without incorporating into the filament polymer. Such IF-binding proteins may include nanobody proteins, intracellularly expressible antibodies or antibody fragments, or IF-binding domains derived from IF-associated proteins or cytolinker proteins that associate with intermediate filaments through affinity-based binding. In such embodiments, the IF-binding protein localizes the self-labeling protein to IFs, thereby positioning the ligand-modified photosensitizer in close proximity to the IF network for light-activated IF disassembly.

In some embodiments, the IF-binding domain is derived from members of the plakin and spectraplakin families of cytolinker proteins, which are characterized by modular architectures capable of associating intermediate filaments with other cellular structures. By way of example, a plectin-derived IF-binding domain may be used to associate with intermediate filaments, including vimentin, neurofilament proteins, and glial fibrillary acidic protein (GFAP). In other embodiments, a desmoplakin-derived IF-binding domain may be used to associate with keratin and/or desmin filament systems. Additional non-limiting examples of plakin/spectraplakin proteins reported to include IF-association or IF-binding regions include dystonin (also referred to as bullous pemphigoid antigen 1 (BPAG1)), envoplakin, and periplakin, including isoforms that associate with keratin-rich epithelial structures.

Beyond plakin and spectraplakin family members, IF-binding domains may be derived from other IF-associated proteins that are reported to interact with intermediate filaments in particular cell types or contexts. Non-limiting examples include keratin-associated proteins in differentiated epithelia (e.g., proteins involved in keratin network organization), IF-associated proteins in muscle and glial cells, including proteins reported to associate with vimentin- and/or desmin-containing filament systems, and large cytolinkers and scaffolding proteins reported to exhibit IF-association capabilities. The foregoing examples are provided for illustrative purposes and not limitation, and additional IF-binding peptides, protein domains, and binding proteins may be used as IF-targeting proteins in accordance with the presently disclosed subject matter.

In some embodiments, the IF-targeting protein is an IF nanobody protein. IF nanobody proteins are single-domain antibody-derived binding proteins that specifically bind to an IF protein and are capable of functioning within the intracellular environment. When used as part of a fusion protein with a self-labeling protein, an IF nanobody protein directs the self-labeling protein, and thus the ligand-bound photosensitizer, to endogenous IF networks without requiring genetic modification or tagging of the IF protein itself.

IF-binding protein-based targeting enables photochemical disassembly of native intermediate filaments while preserving the native composition and expression levels of IF subunits. In such embodiments, the fusion protein may reversibly associate with IFs prior to light activation and need not perturb IF assembly or dynamics under non-activating conditions. Upon administration of light, localized activation of the photosensitizer at the IF surface induces disassembly of the IF network in a manner comparable to embodiments employing IF protein-based targeting.

### Self-Labeling Protein

In addition to the IF-targeting Protein, the fusion protein comprises a self-labeling protein derived from a haloalkane dehalogenase. Such self-labeling proteins specifically bind a ligand that covalently associates with the self-labeling protein, thereby enabling stable attachment of a photosensitizer to the fusion protein. By anchoring the photosensitizer to a self-labeling protein derived from a haloalkane dehalogenase, the presently disclosed subject matter enables precise and reproducible positioning of the photosensitizer relative to the intermediate filament network, while maintaining compatibility with a wide range of ligand chemistries and illumination conditions.

Self-labeling proteins derived from haloalkane dehalogenase are known in the art and include engineered variants capable of covalently binding chloroalkane-containing ligands. Examples of self-labeling proteins derived from haloalkane dehalogenase include, but are not limited to, self-labeling proteins referred to as HaloTag® proteins. The nucleotide sequences encoding and the amino acid sequences for HaloTag® proteins are available in a number of databases that are accessible to the public, including, Addgene, Snap Gene, and GenBank. Additionally, HaloTag® sequence information can be found, for example, in the NCBI Gene Expression Omnibus (GEO) under accession number GSE122542. Examples of amino acids for self-labeling proteins derived from haloalkane dehalogenase include, but are not limited to SEQ ID NO: 1, SEQ ID NO: 2, and functional fragments and variants thereof. Functional fragments or functional variants of SEQ ID NO: 1 or SEQ ID NO: 2 may include proteins having one or more amino acid substitutions, deletions, or insertions relative to the reference sequence, provided that the ability to specifically bind the ligand is retained. Such variants may be engineered to modify properties such as amino acid composition, chemical reactivity, stability, or intracellular performance, without altering the self-labeling activity required to localize the photosensitizer to the IF-targeting protein. In some embodiments, functional variants may lack one or more residues that are not required for ligand binding, including variants designed to remove cysteine residues or other reactive side chains. A fragment or variant is functional if it retains the ability to specifically bind a HaloTag® ligand. HaloTag® ligands are also known in the art, including the following structures:

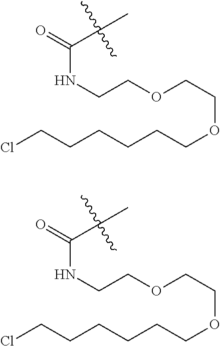 [SMILES: *C(=O)NCCOCCOCCCCCCCl ; *C(=O)NCCOCCOCCCCCCCl]

Additional details about HaloTag® protein and ligands can be found in the Technical Manual entitled “HaloTag® Technology: Aqueous-Soluble Ligands” published by Promega Corporation (Madison, WI) and available at: www.promega.com/resources/protocols/technical-manuals/500/halotag-technology-aqueous-soluble-ligands-protocol/, which is incorporated herein by reference for its disclosure of HaloTag® proteins and ligands, and handling protocols.

### Fusion Protein, Expression, and Introduction

In some embodiments, the fusion protein comprises the self-labeling protein positioned at the C-terminus of the IF-targeting protein. In such configurations, the IF-targeting protein directs the fusion protein to IFs, while the C-terminally positioned self-labeling protein provides an accessible site for ligand binding and photosensitizer attachment.

Positioning the self-labeling protein at the C-terminus may facilitate efficient incorporation of the IF-targeting protein into IF polymers or effective binding to IFs, while minimizing interference with IF assembly, filament architecture, or binding interactions under non-activating conditions. In embodiments where the IF-targeting protein is an IF protein, C-terminal placement of the self-labeling protein may take advantage of the intrinsic flexibility of IF tail domains to allow the self-labeling protein to reside near the filament surface. In embodiments where the IF-targeting protein is an IF-binding protein, C-terminal placement may similarly orient the self-labeling protein toward the IF network.

Unless otherwise indicated, the presently disclosed subject matter does not require that the self-labeling protein be positioned at a particular distance from the IF, provided that the fusion protein localizes the self-labeling protein sufficiently close to the IF to permit light-activated induction of IF disassembly.

In some embodiments, the fusion protein comprises a linker between the self-labeling protein and the IF-targeting protein. The linker may comprise, for example, between 1 and 25 amino acids and may be selected to provide flexibility, spacing, or conformational freedom between the two protein domains. Linkers may be composed of any suitable amino acid sequence and may include flexible linkers, semi-rigid linkers, or combinations thereof.

The presence of a linker may facilitate proper folding and function of the self-labeling protein and the IF-targeting protein, reduce steric hindrance, and allow the self-labeling protein to adopt an orientation favorable for ligand binding and photosensitizer activity. In some embodiments, the linker length and composition are selected to permit the photosensitizer, once bound to the self-labeling protein, to reside within effective proximity of the intermediate filament surface while maintaining normal IF organization prior to light activation.

The presently disclosed subject matter does not require a particular linker sequence, provided that the fusion protein retains the ability to localize the self-labeling protein to IFs and to bind the ligand. Accordingly, fusion proteins with linkers of varying length and composition are contemplated, including fusion proteins in which the linker is optimized for a given IF system, cell type, or experimental context.

In some embodiments, the fusion protein is expressed from a nucleic acid introduced into the cell or tissue. The nucleic acid encodes the fusion protein comprising the self-labeling protein and the IF-targeting protein, and may include regulatory elements that permit expression in the cell or tissue of interest. Introduction of the nucleic acid may result in transient or stable expression of the fusion protein and may be performed prior to administration of the photosensitizer.

Expression from an introduced nucleic acid enables production of the fusion protein within the cellular environment, allowing the fusion protein to localize to intermediate filaments through incorporation into IF polymers or binding to IF proteins. The presently disclosed subject matter does not require that the fusion protein be expressed at a particular level, provided that sufficient fusion protein is present to permit localization of the photosensitizer to the IF network and subsequent light-induced disassembly.

In some embodiments, the fusion protein is introduced into the cell or tissue as a pre-formed polypeptide. In such embodiments, the fusion protein may be produced and purified ex vivo and subsequently delivered to the cell or tissue using methods known in the art. Introduction of the fusion protein as a protein does not require transcription or translation within the target cell and may be used in contexts where nucleic acid delivery or expression is undesirable or impractical.

Following introduction, the fusion protein may localize to intermediate filaments through incorporation into IF polymers or through specific binding interactions, depending on the IF-targeting protein employed. Once localized, the self-labeling protein domain of the fusion protein remains capable of binding the ligand-modified photosensitizer, thereby enabling light-activated induction of IF disassembly.

In some embodiments, the fusion protein is expressed via a viral vector. Viral vectors may be used to deliver a nucleic acid encoding the fusion protein to the cell or tissue and to drive expression of the fusion protein in a wide range of cell types. Viral vector-mediated expression may be employed in cultured cells, tissues, or in vivo in a live animal.

Use of viral vectors may enable efficient delivery and expression of the fusion protein in dividing or non-dividing cells and may support localized or systemic expression depending on the vector and route of administration. The presently disclosed subject matter does not require a particular viral vector type, provided that the vector is capable of delivering and expressing the nucleic acid encoding the fusion protein in the target cell or tissue.

In some embodiments, the fusion protein is expressed from a knock-in genomic locus encoding an endogenous IF protein. In such embodiments, a nucleotide sequence encoding the self-labeling protein is inserted into the genome at or near an endogenous IF gene, resulting in expression of the fusion protein under control of the endogenous regulatory elements. This approach enables expression of the fusion protein at physiological levels and in native spatial and temporal patterns.

Expression from a knock-in genomic locus allows the fusion protein to be produced as part of the endogenous IF protein population and to incorporate into native IF networks without the need for overexpression. In such embodiments, the fusion protein may coexist with unmodified IF proteins and may be expressed in a heterozygous or homozygous manner. The presently disclosed subject matter is applicable to cells, tissues, or organisms in which the knock-in modification has been introduced and does not require further genetic manipulation prior to photosensitizer labeling and light activation.

The expression and introduction strategies described herein may be selected based on the experimental context, cell or tissue type, and desired level of control over fusion protein expression. The presently disclosed subject matter does not require a single mode of expression or introduction, and different strategies may be used interchangeably or in combination, provided that the fusion protein localizes the self-labeling protein to intermediate filaments and permits ligand binding and light-activated IF disassembly.

### Ligand-Modified Photosensitizers

In some embodiments of the presently disclosed subject matter, the photosensitizer is a rhodamine-derived photosensitizer. Rhodamine-derived photosensitizers include compounds based on a rhodamine scaffold that are capable of absorbing light and undergoing light-dependent photochemical reactions when excited. Such compounds may function as fluorescent labels under low-intensity illumination and as photosensitizers under illumination conditions sufficient to induce photochemical activity.

Rhodamine-derived photosensitizers may be modified with a ligand that specifically binds to the self-labeling protein, thereby enabling covalent association of the photosensitizer with the fusion protein and localization of the photosensitizer to the intermediate filament network. The presently disclosed subject matter does not require that the rhodamine-derived photosensitizer be optimized exclusively for photosensitization, provided that the compound exhibits light-dependent activity when localized to the IF.

As will be appreciated by one of ordinary skill in the art, rhodamine-derived photosensitizers are photosensitive compounds based on the rhodamine molecular structure, which is commonly used in fluorescence and imaging applications. Rhodamine-derived photosensitizers are capable of absorbing light and transferring energy to molecular oxygen or other substrates, thereby generating reactive species such as singlet oxygen (¹O₂) or free radicals.

Compounds based on the rhodamine molecular structure typically include a xanthene core comprising a fused tricyclic system with a phenyl ring extension at the 9-position of the central xanthene ring. The carbons of the phenyl ring extension are designated C1 for the carbon directly attached to the xanthene core, followed by C2′, C3′, C4′, C5′, and C6′ in a clockwise orientation. In some embodiments, a rhodamine-derived photosensitizer is modified with a ligand at the C3′, C4′, C5′, or C6′ position of the phenyl ring extension.

Examples of rhodamine-derived photosensitizers suitable for use in accordance with this disclosure include, but are not limited to, those described in the Examples herein, as well as photosensitizers disclosed in U.S. Pat. Nos. 9,933,417; 10,018,624; 10,161,932; 10,495,632; 11,787,946; and 12,344,594, and U.S. Patent Application Publication No. US 2024-0239818, each of which is incorporated herein by reference in its entirety for its disclosure of photosensitizer compounds and derivatives.

In some embodiments, the photosensitizer comprises a heavy-atom-substituted rhodamine. Heavy-atom substitution may include incorporation of halogens or other heavy atoms into the rhodamine scaffold, which can enhance intersystem crossing and increase the efficiency of light-induced photochemical reactions.

Use of heavy-atom-substituted rhodamine photosensitizers may enable efficient IF disassembly at lower light doses or with shorter illumination times compared to non-substituted rhodamine compounds. The presently disclosed subject matter does not require a particular heavy atom or substitution pattern, provided that the photosensitizer retains the ability to bind the self-labeling protein via the ligand and to mediate light-dependent activity when localized to the IF.

In some embodiments of the presently disclosed subject matter, the photosensitizer is a fluorescein-derived photosensitizer. Fluorescein-derived photosensitizers include compounds based on a fluorescein or carbofluorescein scaffold that are capable of absorbing light and mediating photochemical reactions upon excitation. Such photosensitizers may be selected based on their excitation spectra, photostability, or compatibility with other imaging or labeling modalities.

In some embodiments of the presently disclosed subject matter, the photosensitizer is selected to generate reactive oxygen species (ROS) upon excitation with light. Such reactive oxygen species may include singlet oxygen, free radicals, or other short-lived reactive species capable of mediating localized chemical modifications. When generated in proximity to the IF network, these reactive species may induce disassembly of IFs through localized photochemical effects.

The presently disclosed subject matter does not require that ROS generation be global or detectable throughout the cell or tissue. Rather, ROS generation may be highly localized to the vicinity of the photosensitizer bound to the fusion protein, thereby enabling selective IF disassembly without inducing widespread oxidative damage. Selection of the photosensitizer may be based on its ability to generate ROS under defined illumination conditions while remaining compatible with the desired spatial and temporal control.

Regardless of the photosensitizer that is selected, it may be modified with a ligand that specifically binds to the self-labeling protein. Upon ligand binding, the photosensitizer is positioned in proximity to the intermediate filament network, enabling light-activated induction of IF disassembly. The presently disclosed subject matter encompasses the use of fluorescein-derived compounds that function as conventional fluorescent dyes under non-activating conditions and as effective photosensitizers under activating illumination.

In some embodiments of the presently disclosed subject matter, the ligand that specifically binds to the self-labeling protein comprises a defined chemical structure capable of covalently associating with the self-labeling protein. Such ligands may include chloroalkane-containing ligands or other ligand chemistries known to bind self-labeling proteins derived from haloalkane dehalogenase.

Modification of the photosensitizer with the ligand enables stable and specific attachment of the photosensitizer to the self-labeling protein of the fusion protein. This ligand-mediated association localizes the photosensitizer to the IF network through the IF-targeting protein and positions the photosensitizer for effective light-activated activity. Unless otherwise indicated, the presently disclosed subject matter does not require that the ligand have a particular linker length, orientation, or substitution pattern, provided that it permits covalent binding to the self-labeling protein.

The photosensitizer chemistries described herein may be selected independently of the IF-targeting strategy, mode of fusion protein expression, or illumination configuration. Rhodamine-derived, fluorescein-derived, heavy-atom-substituted, and ROS-generating photosensitizers are all compatible with ligand-mediated targeting to self-labeling proteins and light-activated induction of IF disassembly. Accordingly, the presently disclosed subject matter encompasses a broad class of photosensitizers that, when localized to IFs and activated by light, are capable of inducing controlled IF disassembly.

### Light Administration

In some embodiments of the presently disclosed subject matter, the light administered to the cell or tissue comprises laser illumination. Laser illumination may be used to deliver light of a defined wavelength, intensity, and spatial distribution to activate the photosensitizer bound to the fusion protein. Lasers may be operated in continuous or pulsed modes and may be configured to illuminate an entire field of view or a localized region within a cell or tissue.

Use of laser illumination may facilitate precise temporal control over photosensitizer activation and enable spatially confined induction of intermediate filament disassembly. Unless otherwise indicated, the presently disclosed subject matter does not require a particular laser configuration, provided that the administered light is capable of exciting the photosensitizer when localized to the intermediate filament network.

In some embodiments of the presently disclosed subject matter, the light administered to the cell or tissue has a wavelength between about 500 nm and about 700 nm. This wavelength range encompasses excitation wavelengths suitable for activating rhodamine-derived and fluorescein-derived photosensitizers, including photosensitizers that absorb in the green, orange, red, or far-red regions of the visible spectrum.

In some embodiments, the light has a wavelength selected from about 561 nm or about 639 nm. Such wavelengths may be selected based on the excitation characteristics of the photosensitizer and the optical configuration used. Illumination at these wavelengths may permit efficient activation of the photosensitizer while maintaining compatibility with common fluorescence microscopy platforms. The presently disclosed subject matter does not require that the wavelength be restricted to a single value, and illumination at multiple wavelengths or over a wavelength range is contemplated, provided that the photosensitizer is excited under the administered conditions.

In some embodiments of the presently disclosed subject matter, the light is administered at an energy dose sufficient to induce disassembly of the intermediate filament without inducing global cellular phototoxicity. In such embodiments, illumination conditions are selected to activate the photosensitizer localized to the IF network while avoiding widespread cellular damage or disruption of non-targeted cellular structures.

As will be appreciated by those of ordinary skill in the art upon study of this disclosure, when light is administered, the wavelength of the light may be selected to correspond to, or overlap with, the excitation spectrum of the photosensitizer being used, such as a rhodamine-derived or fluorescein-derived photosensitizer.

A variety of light sources suitable for exciting photosensitizers are known in the art and may be employed. In some embodiments, light is provided by one or more lasers configured to emit light at a selected wavelength or wavelength range. In other embodiments, light may be provided by light-emitting diodes (LEDs), arc lamps (such as short-arc mercury lamps), or other illumination systems capable of delivering light of sufficient intensity and appropriate spectral characteristics to excite the photosensitizer.

The intensity, duration, and spatial distribution of the administered light may be selected based on the photosensitizer used, the cell or tissue being illuminated, and the desired extent or localization of the photochemical effect. In contrast, light sources typically used for transmitted-light imaging, ambient room lighting, or other low-intensity illumination are generally insufficient to substantially excite the photosensitizers described herein to produce the light-dependent effects disclosed.

Unless otherwise indicated, the particular light source or illumination configuration is not critical, provided that light is administered under conditions sufficient to excite the photosensitizer when localized to the self-labeling protein and associated intermediate filament.

The energy dose may be controlled by adjusting parameters such as light intensity, exposure time, repetition, and spatial extent of illumination. Because the photosensitizer is localized to the IF via the fusion protein, localized photochemical activity may be sufficient to induce IF disassembly at lower overall energy doses than would be required for non-targeted illumination. Accordingly, the presently disclosed subject matter enables selective IF disassembly while preserving overall cell viability and function under appropriate conditions.

Unless otherwise indicated, the presently disclosed subject matter does not require complete avoidance of photochemical effects outside the IF, provided that the administered light does not result in global phototoxicity that would obscure or confound the effects of IF disassembly.

As will be appreciated by those of ordinary skill in the art upon study of this disclosure, suitable modes and locations of administration of the photosensitizer may depend on a variety of factors, including the cell or tissue of interest, whether the cell or tissue is in vitro, ex vivo, or in vivo, whether administration is performed in a live animal, and the anatomical location within the animal.

Modes of administration include, but are not limited to, injection, topical application, perfusion, incubation, or other delivery approaches known in the art. In some embodiments, for example when administration is performed in cultured cells or tissues, a photosensitizer may be added to a culture medium and, following an incubation period sufficient to permit labeling, excess photosensitizer may be removed, for example by washing.

In other embodiments, for example when administration is performed in vivo in a live animal, the photosensitizer may be administered by injection, including systemic or localized injection. By way of example, in a rodent model, systemic administration may include intravenous injection, such as tail vein injection, while localized administration may include injection into or near a tissue of interest.

In some embodiments, particularly for accessible or surface tissues, administration may comprise topical application or incubation with the photosensitizer, allowing for passive uptake and labeling of cells or tissues. Unless otherwise indicated, the particular mode of administration is not critical, provided that the photosensitizer is delivered in a manner sufficient to permit binding to the self-labeling protein and subsequent light-dependent activity.

The light administration parameters described herein may be selected independently of the IF-targeting strategy, fusion protein architecture, or photosensitizer chemistry. Laser-based illumination, visible-wavelength excitation, and controlled energy dosing are all compatible with global or localized induction of IF disassembly. Accordingly, the presently disclosed subject matter encompasses a wide range of illumination configurations that activate a photosensitizer localized to IFs and induce controlled IF disassembly upon light exposure.

### Spatial and Temporal Control

In some embodiments of the presently disclosed subject matter, the light is administered to a localized subcellular region of the cell. Localized administration of light may be achieved by restricting illumination to a defined region within the cell or tissue, such as a portion of the cytoplasm, a perinuclear region, a cellular process, or another subcellular compartment of interest. In such embodiments, activation of the photosensitizer occurs selectively within the illuminated region, while regions outside the illuminated area are not exposed to activating light.

Localized light administration enables spatially confined activation of the photosensitizer bound to the fusion protein and permits targeted disassembly of intermediate filaments within a defined subcellular region. The size, shape, and position of the illuminated region may be selected based on the experimental or biological context and may be adjusted to achieve the desired degree of spatial resolution.

In some embodiments of the presently disclosed subject matter, disassembly of the intermediate filament is induced locally within an illuminated region, while intermediate filaments outside the illuminated region remain intact. In such embodiments, the photosensitizer is activated only where light is administered, resulting in spatially restricted photochemical activity and localized IF disassembly.

This localized disassembly may include fragmentation, severing, or loss of filament continuity within the illuminated region, while IF networks in non-illuminated regions retain their pre-existing organization. Such spatial confinement enables selective perturbation of intermediate filament architecture without requiring global disruption of the IF network throughout the entire cell or tissue. The presently disclosed subject matter thus allows comparison of filament behavior, cellular responses, or mechanical properties between regions with intact IFs and regions in which IFs have been locally disassembled.

In some embodiments of the presently disclosed subject matter, the light is administered repeatedly over multiple illumination cycles. Repeated illumination may involve multiple exposures separated by defined intervals, continuous illumination over a series of frames, or a combination thereof. Repeated administration of light may be used to gradually induce IF disassembly, to maintain photosensitizer activation over time, or to achieve a desired extent of filament disruption.

The number, duration, and spacing of illumination cycles may be selected based on the photosensitizer used, the cellular context, and the desired temporal dynamics of IF disassembly. Repeated illumination cycles may be applied globally or locally and may be adjusted to permit monitoring of intermediate filament behavior before, during, and after disassembly. The presently disclosed subject matter does not require that disassembly occur in a single illumination event, and progressive or staged disassembly over multiple cycles is contemplated.

The spatial and temporal control features described herein may be used independently or in combination with other aspects of the presently disclosed subject matter, including the choice of IF-targeting protein, photosensitizer chemistry, and light administration parameters. Localized illumination, preservation of non-illuminated IFs, and repeated illumination cycles enable precise control over when and where intermediate filament disassembly occurs, providing flexibility for investigating IF function and dynamics within complex cellular or tissue environments.

### Disassembly and Specificity

In some embodiments of the presently disclosed subject matter, disassembly of the intermediate filament occurs without detectable disassembly of actin filaments or microtubules. In such embodiments, activation of the photosensitizer localized to the intermediate filament network induces selective disruption of IF structure while leaving other major cytoskeletal systems substantially intact.

This selectivity may arise from localization of the photosensitizer to intermediate filaments via the fusion protein and from intrinsic differences in susceptibility of cytoskeletal polymers to localized photochemical activity. Accordingly, the presently disclosed subject matter enables targeted perturbation of intermediate filaments without requiring concurrent destabilization of actin filaments or microtubules and without inducing global cytoskeletal collapse. Preservation of actin and microtubule organization may allow cellular processes dependent on these systems to continue during or after IF disassembly.

Unless otherwise indicated, the absence of detectable disassembly refers to the lack of gross fragmentation or loss of structural integrity of actin filaments or microtubules under conditions sufficient to induce IF disassembly, and does not preclude subtle or indirect cellular responses to IF perturbation.

In some embodiments of the presently disclosed subject matter, disassembly of the IF is accompanied by fragmentation of IF polymers into discrete IF fragments. Such fragmentation may include severing of filaments, breakdown of filament continuity, formation of shortened filament segments, or generation of punctate or particulate IF structures within the cell or tissue.

Fragmented IF polymers may be insoluble or partially insoluble and may persist within the cytoplasm or tissue following light activation. In some embodiments, fragmentation results in a redistribution of IF material from an extended filamentous network into dispersed fragments or aggregates. The presently disclosed subject matter does not require complete depolymerization of IFs into monomeric subunits, and fragmentation may occur without full dissolution of all filament material.

Fragmentation of IF polymers may occur locally within an illuminated region or globally throughout the cell or tissue, depending on the illumination conditions used. Such fragmentation provides a distinct and observable outcome of IF disassembly that is separable from mere reorganization or relocalization of intact filaments.

In some embodiments of the presently disclosed subject matter, disassembly of the intermediate filament is irreversible over a period of at least several hours. In such embodiments, IF fragmentation induced by light activation persists beyond the illumination period and is not rapidly reversed by cessation of light exposure or removal of the photosensitizer.

Irreversibility may include persistence of fragmented IF polymers, failure of fragmented IFs to reassemble into an extended filamentous network, or delayed recovery of filament organization over a period of hours. In some embodiments, reestablishment of a filamentous IF network, if it occurs, may require de novo synthesis of IF proteins, cellular remodeling, or extended recovery periods.

The presently disclosed subject matter does not require permanent elimination of IF proteins or irreversible cellular damage. Rather, the term “irreversible” refers to the absence of rapid or spontaneous reassembly of the IF network on short timescales following light-induced disassembly. This temporal persistence enables investigation of downstream cellular responses to sustained loss of IF integrity without continuous illumination or ongoing photosensitizer activation.

The biological outcomes described herein, including selective IF disassembly, fragmentation into IF polymers or fragments, and persistence of the disassembled state, may occur independently or in combination, depending on the IF system, photosensitizer chemistry, and illumination parameters employed. These outcomes distinguish the presently disclosed subject matter from non-specific photodamage and provide a controlled means of perturbing IF architecture while preserving overall cellular viability and structure under appropriate conditions.

### Cells and Tissue

In some embodiments of the presently disclosed subject matter, the cell is a mammalian cell. Mammalian cells include cells derived from human or non-human mammals and may be primary cells, immortalized cell lines, stem cells, differentiated cells, or progenitor cells. The presently disclosed subject matter may be used in mammalian cells cultured in vitro, maintained ex vivo, or present within a tissue or organism.

Use of mammalian cells enables investigation or manipulation of intermediate filament systems that are characteristic of mammalian cytoskeletal organization, including type I, type II, type III, and type IV intermediate filaments. The presently disclosed subject matter does not require a particular mammalian species or cell lineage, provided that the cell expresses, assembles, or contains intermediate filaments that can be targeted by the fusion protein and subjected to light-activated disassembly.

In some embodiments of the presently disclosed subject matter, the tissue is a live tissue in an animal. The tissue may be maintained ex vivo or may be within a living organism. Live tissues may include, without limitation, nervous tissue, muscle tissue, connective tissue, epithelial tissue, or combinations thereof.

When performed in a live tissue or organism, the method may involve expressing or introducing the fusion protein in cells within the tissue, administering the photosensitizer to the tissue or organism, and administering light to the tissue under conditions effective to induce intermediate filament disassembly. Light may be delivered locally to a region of interest within the tissue or more broadly, depending on the desired extent of IF disassembly.

The presently disclosed subject matter does not require that all cells within the tissue be modified or illuminated, and selective targeting of subsets of cells or regions within a tissue is contemplated. Accordingly, the presently disclosed subject matter is applicable to in vivo studies of intermediate filament function and dynamics in physiologically relevant tissue environments.

In some embodiments of the presently disclosed subject matter, the IF is a cytoplasmic intermediate filament. Cytoplasmic intermediate filaments include intermediate filament systems that assemble in the cytoplasm and contribute to cellular architecture, mechanical resilience, and intracellular organization.

Examples of cytoplasmic intermediate filaments include, but are not limited to, vimentin filaments, desmin filaments, glial fibrillary acidic protein filaments, peripherin filaments, keratin filaments, nestin filaments, and neurofilaments. Such filaments may form homopolymeric or heteropolymeric networks and may interact with other cytoskeletal systems, organelles, or cellular structures.

By targeting cytoplasmic intermediate filaments, the presently disclosed subject matter enables selective disassembly of filament networks that play central roles in cytoplasmic organization without requiring disruption of nuclear intermediate filaments or other nuclear structures. Unless otherwise indicated, the presently disclosed subject matter does not exclude application to other intermediate filament classes, provided that the fusion protein and photosensitizer can be localized to the filament system of interest.

The cell and tissue contexts described herein may be selected independently of the IF-targeting strategy, photosensitizer chemistry, or light administration parameters. The presently disclosed subject matter is applicable to mammalian cells, live tissues, and cytoplasmic intermediate filament systems across a wide range of biological settings, enabling controlled induction of IF disassembly in physiologically relevant environments.

### Representative Tissues and Cellular Contexts

As will be appreciated by those of ordinary skill in the art upon study of this disclosure, relevant tissues of interest may vary depending on the particular IF protein being targeted, the biological context, and whether the cell or tissue is studied in vitro, ex vivo, or in vivo.

By way of example, glial fibrillary acidic protein (GFAP) is predominantly expressed in the central nervous system and is particularly associated with astrocytes in the brain, making brain tissue and astrocyte-containing regions relevant in embodiments involving GFAP. Vimentin is widely expressed during development across numerous tissues and, in adult organisms, is primarily associated with mesenchymal cell types, including fibroblasts, endothelial cells, and certain immune cells, rendering a broad range of tissues relevant for vimentin-based embodiments.

Desmin is predominantly expressed in muscle tissues, including skeletal muscle, cardiac muscle, and smooth muscle, and therefore muscle tissues are relevant in embodiments involving desmin. Peripherin is expressed in peripheral sensory neurons as well as in certain motor neurons of the central nervous system, including spinal motor neurons, making both peripheral nervous system tissues and selected central nervous system regions relevant in embodiments involving peripherin.

Keratins are expressed in a wide variety of epithelial tissues throughout the body, including but not limited to epithelia of the skin, lung, liver, and gastrointestinal tract, and epithelial tissues are therefore relevant in embodiments involving keratin intermediate filaments.

Unless otherwise indicated, the identification of relevant tissues is provided by way of example and not limitation, and intermediate filament proteins may be present in additional cell types or tissues depending on developmental stage, physiological state, or pathological condition.

While the terms used herein are believed to be well understood by those of ordinary skill in the art, certain definitions are set forth to facilitate explanation of the presently disclosed subject matter.

Unless defined otherwise, all technical and scientific terms used herein have the same meaning as is commonly understood by one of skill in the art to which the invention(s) belong.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUBMB Joint Commission on Biochemical Nomenclature (See, iubmb.qmul.ac.uk/).

Although any methods, devices, and materials similar or equivalent to those described herein can be used in the practice or testing of the presently disclosed subject matter, representative methods, devices, and materials are described herein.

In certain instances, nucleotides and polypeptides disclosed herein are included in publicly available databases, such as NCBI® Gene (also known as Entrez Gene), GENBANK® and UNIPROT®. Unless otherwise indicated or apparent the references to such publicly available databases are references to the most recent version of the database as of the filing date of this Application.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

As used herein, the term “disassembly,” when referring to an intermediate filament (IF), refers to a loss of filament integrity characterized by fragmentation, severing, breakdown, or dissolution of an assembled intermediate filament or intermediate filament network into shorter filament fragments, non-continuous filament structures, and/or soluble or partially soluble intermediate filament subunits. Disassembly may include, but does not require, complete depolymerization of the intermediate filament, and encompasses partial or localized disruption of filament structure, including severing of individual filaments, fragmentation of filament bundles, loss of filament continuity, redistribution of filament material, or collapse of an extended filament network. Disassembly may occur globally within a cell or tissue, or locally within a defined subcellular region, and may be rapid or progressive over time. Unless otherwise indicated, disassembly does not require irreversible destruction of intermediate filament proteins and does not require complete elimination of all filament-derived material from the cell or tissue.

As used herein, the term “FilaBuster” is sometimes used as a convenient shorthand to refer to embodiments of the methods and systems described in this disclosure in which a photosensitizer is localized to IFs via a self-labeling protein and activated by light to induce intermediate filament disassembly. The term “FilaBuster” is used for ease of reference and descriptive convenience and does not denote a separate invention, limitation, or required feature beyond those expressly recited in the claims. Unless otherwise indicated, references to “FilaBuster” encompass methods, kits, compositions, illumination regimes, and experimental conditions consistent with the disclosed subject matter, including global or localized illumination, different photosensitizer chemistries, and different intermediate filament targeting strategies. Use of the term “FilaBuster” is not intended to limit the scope of the claims, which are defined solely by their recited elements.

As used herein, the terms “functional fragment” and “functional variant,” when referring to a reference self-labeling protein, mean a fragment or variant of a reference self-labeling protein that retains the ability to specifically bind a ligand that covalently associates with the self-labeling protein. As used herein, the terms “functional fragment” and “functional variant,” when referring to a reference IF protein, mean a fragment or variant of a reference IF protein that retains the ability the ability to participate in intermediate filament assembly or to associate with assembled intermediate filaments. Unless otherwise indicated, functional fragments and functional variants are not required to be identical in sequence length, amino acid composition, or physicochemical properties to the reference protein. A fragment of a reference protein may comprise a truncated portion of the reference protein, with one or more amino acids removed from the N-terminus and/or the C-terminus. A variant of a reference protein may comprise one or more amino acid substitutions, deletions, insertions, or combinations thereof relative to the reference protein, including conservative or non-conservative substitutions. Variants can include naturally occurring variants, engineered variants, and modified versions of the reference protein, including variants designed to alter chemical reactivity, stability, or amino acid composition.

As used herein, the term “fusion protein” refers to a single polypeptide comprising two or more protein domains that are operably linked such that the domains are translated as part of the same polypeptide chain. In the context of this disclosure, a fusion protein may comprise a self-labeling protein and an IF-targeting protein. The protein domains of the fusion protein may be directly joined or may be separated by one or more linker sequences, which may vary in length and amino acid composition. A fusion protein may be expressed in a cell or tissue from a nucleic acid encoding the fusion protein or may be introduced into a cell or tissue as a pre-formed polypeptide. Expression or introduction of the fusion protein may occur in cultured cells, tissues, or in a live animal. In some embodiments, a fusion protein is expressed from a genetically modified organism, including a knock-in animal in which a nucleotide sequence encoding the fusion protein is inserted into a genome and expressed in vivo. In other embodiments, a nucleotide encoding the fusion protein is delivered to a cell or tissue using methods known in the art, including viral vectors, lipid-based transfection, nanoparticle delivery, microinjection, genome-editing methods, or combinations thereof. In still other embodiments, the fusion protein is purified and delivered directly to a cell or tissue as a protein. Unless otherwise indicated, a fusion protein is not required to preserve the native tertiary structure, subcellular localization, or oligomerization state of each individual domain, provided that the fusion protein retains the ability to localize to intermediate filaments through the IF protein or IF nanobody protein domain and to specifically bind a ligand through the self-labeling protein domain. The term “fusion protein” includes full-length fusion constructs as well as functional fragments or functional variants thereof.

As used herein, the term “intermediate filament” or “IF” refers to a cytoskeletal polymeric structure composed of one or more IF proteins. These proteins assemble into elongated, apolar filaments with a characteristic diameter between that of actin filaments and microtubules. Found in both cytoplasmic and nuclear systems, IFs may be homopolymeric or heteropolymeric. The assembly process involves higher-order multimeric complexes; for instance, type III IF proteins form tetrameric subunits that further associate into mature filaments.

Exemplary IF proteins include vimentin, desmin, glial fibrillary acidic protein (GFAP), peripherin, keratins (types I and II), nestin, neurofilament proteins, and nuclear lamins. As will be appreciated by the skilled artisan, nucleotide sequences encoding IF proteins and the corresponding amino acid sequences are publicly available in biological sequence databases, including GenBank, OMIM, and UniProt. By way of example, IF protein sequence information can be found under the following accession numbers: Vimentin (VIM)-Nucleotide Sequence: NM_003380 (NCBI GenBank), Amino Acid Sequence: NP_003371 (NCBI GenBank); Desmin (DES)-Nucleotide Sequence: NM_001927 (NCBI GenBank), Amino Acid Sequence: NP_001918 (NCBI GenBank); Peripherin (PRPH)-Nucleotide Sequence: NM_006262 (NCBI GenBank), Amino Acid Sequence: NP_006253 (NCBI GenBank); and Glial Fibrillary Acidic Protein (GFAP)-Nucleotide Sequence: NM_002055 (NCBI GenBank), Amino Acid Sequence: NP_002046 (NCBI GenBank).

Physically, an IF may exist as part of an extended intracellular filament network, a filament bundle, or a localized filament assembly within a cell or tissue. IFs may be present within individual cells (intracellular IFs), within multicellular tissues, or associated with tissue-level architectures formed by multiple cells. In addition to intracellular IFs, intermediate filament proteins and filamentous IF structures may be released from cells or secreted into the extracellular environment under certain physiological or pathological conditions, including during cell damage, stress, remodeling, or turnover. Accordingly, the term “intermediate filament” as used herein also encompasses IFs or IF-derived structures that are present outside of cells, including extracellular, pericellular, or tissue-associated IFs. The term “intermediate filament” encompasses fully assembled filaments, partially assembled filaments, filament bundles, and mixed IF networks comprising multiple IF protein species. Unless otherwise indicated, “intermediate filament” refers to the assembled filamentous form rather than solely to monomeric, dimeric, tetrameric, or soluble subunit precursors thereof.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “photosensitizer” refers to a chemical compound that, upon exposure to light of an appropriate wavelength, enters an excited state and produces a light-dependent photochemical effect capable of affecting nearby molecules or structures. Photosensitizers include fluorescent and non-fluorescent compounds capable of undergoing light-induced photochemistry.

As used herein, a “self-labeling protein” is a protein that is capable of covalently attaching to a specific chemical substrate, referred to as a “ligand” or “self-labeling protein ligand,” thereby forming a stable covalent bond between the protein and the ligand. A self-labeling protein ligand is a chemical compound that specifically binds to the self-labeling protein and covalently associates therewith. Such ligands may be linked to, or otherwise comprise, additional chemical moieties or functional groups. Together, the self-labeling protein and its ligand provide a system for facilitating the specific attachment of a compound or functional group to the protein in vitro or within a living cell. The term “self-labeling” indicates that the protein is capable of catalyzing covalent attachment of the ligand without the need for additional enzymes or cofactors.

As used herein, the term “specifically binds” refers to a binding interaction between two molecules in which one molecule preferentially associates with a target molecule relative to non-target molecules under physiological or assay conditions. In the context of this disclosure, “specifically binds” encompasses binding interactions that are sufficient to localize a ligand-modified compound to a target protein or protein complex through selective association, including covalent or non-covalent interactions, as applicable. Specific binding does not require exclusive binding to the target, a particular binding affinity, or the absence of any non-specific interactions, provided that the binding interaction results in preferential association with the target molecule.

As used herein, the term “vector” refers to a nucleic acid construct that is capable of delivering, maintaining, and/or expressing a nucleotide sequence in a cell or tissue. A vector may comprise regulatory elements operably linked to a nucleotide sequence encoding a fusion protein, including promoters, enhancers, transcriptional start sites, polyadenylation signals, and other elements that facilitate transcription and translation in a host cell. A vector may be configured for transient expression, stable expression, or genomic integration. Vectors include, but are not limited to, plasmid vectors, viral vectors, episomal vectors, and genome-editing vectors. Viral vectors may include lentiviral, adenoviral, adeno-associated viral, retroviral, or other viral delivery systems. Genome-editing vectors may include vectors used in conjunction with site-specific nucleases or recombinases. A vector may be introduced into a cell or tissue using methods known in the art, including transfection, transduction, electroporation, microinjection, nanoparticle-based delivery, or combinations thereof. In some embodiments, a vector is used to express a fusion protein in cultured cells, tissues, or in a live animal, including expression from a genetically modified organism such as a knock-in animal. Unless otherwise indicated, the term “vector” encompasses single-vector systems as well as multi-vector systems used together to achieve expression of the fusion protein.

The presently disclosed subject matter is further illustrated by the following specific but non-limiting examples. The following examples may include compilations of data that are representative of data gathered at various times during the course of development and experimentation related to the present invention.

### EXAMPLES

### Example 1: Plasmids

Lentiviral vectors for vimentin-HaloTag® protein, Vimentin-C328A-HaloTag® Protein, vimentin-C328H-HaloTag® protein, vimentin-SnapTag® protein, vimentin-mNeonGreen protein, vimentin-SuperNova2 protein, vimentin-mStayGold protein, desmin-HaloTag® protein, and peripherin-HaloTag® protein were cloned by VectorBuilder. In all cases, the tag was added to the c terminus of human vimentin, desmin, or peripherin via a six residue (GSGSGS (SEQ ID NO: 3)) linker. GFAP-HaloTag® protein (Addgene, 169474) and Vimentin-mEmerald protein (Addgene, 54299) were obtained from Addgene. The vimentin-chromobody (Vimentin-VHH-TagGFP2) was purchased from Chromotek (now ProteinTech, vcg). Vimentin-VHH-HaloTag® protein was generated by synthesizing coHaloTag protein—a cysteine-free variant of HaloTag7 protein with C to T and C to V amino acid substitutions (SEQ ID NO: 2), and subcloning into the Vimentin-VHH-TagGFP2 plasmid using HindIII and NotI restriction sites (genscript). As a result, coHaloTag protein is linked to the c-terminus of the vimentin VHH/nanobody via a 22 amino acid linker (RSLGGGGSGGGGSGGGGSGGGG (SEQ ID NO: 4)). Keratin18-HaloTag® protein was obtained from the (Janelia Cell and Molecular Biology core facility).

### Example 2: Cell Culture

COS-7 cells (African green monkey kidney fibroblast-like cells; ATCC), PTK2 cells (Potorous tridactylus kidney epithelial cells; ATCC), RPE-1 cells (retinal pigment epithelial cells; ATCC), MEF (mouse embryonic fibroblasts; wild-type (WT) and vimentin-null (Vim−/−)), HEK293T cells (human embryonic kidney 293T cells), U2-OS cells (human osteosarcoma cells; ATCC), vimentin-HaloTag® protein knock-in U2-OS cells (courtesy of Stefan Jakobs), and NIH/3T3 cells (mouse embryonic fibroblasts; ATCC) were maintained in Dulbecco's Modified Eagle Medium (DMEM; Corning) with 4.5 g/L glucose supplemented with 10% fetal bovine serum, 2 mM GlutaMAX, 100 U/mL penicillin, and 100 μg/mL streptomycin. Normal human dermal fibroblasts (NHDF; MatTek, NHDF-CRY-NEO) were cultured in NHDF growth medium (MatTek).

All cells were maintained in a 37 C, 5% CO₂ incubator. For imaging experiments, cells were seeded on either 35 mm #1.5 glass-bottom dishes (MatTek, P35G-1.5-C-C) or 12 well #1.5 glass-bottom plates (CellViz; P12-1.5H-N) coated with either fibronectin or matrigel (500 μg/ml). Transient transfection was performed using either FuGene HD & FuGene 4k (Roche). Nucleofection was performed using an Amaxa Nucleofector and Solution SE for COS-7 and U2-OS. Imaging was performed in complete culture medium and cells were maintained at 37° C./5% CO₂ throughout all live imaging experiments. Drug treatments were performed using 10 μM nocodazole diluted in DMSO (Millipore Sigma, M1404), 10 μM taxol diluted in DMSO (Millipore Sigma, PHL89806), or 3 μM Latrunculin A diluted in DMSO. (Millipore Sigma, L5163). Cells were incubated with HaloTag® ligands at 250 nM for 30-60 minutes and washed 3× in complete media before imaging. Cells were incubated with CellROX Deep Red (Invitrogen, C10422) at 5 μM concurrently with HaloTag® Ligand (HTL) labeling.

### Example 3: Stable Cell Lines

Vimentin-mEmerald knock in COS-7: mEmerald was knocked into the C terminal of vimentin in COS-7 cells using CRISPR-Cas9. Guide RNA (gRNA) was designed using CHOPCHOP and screened for off target cutting and efficiency using CCTop. Both gRNA and Alt-R™ S.p. HiFi Cas9 were procured from IDT, while a ssDNA donor and oligo containing a tCTS sequence were synthesized by Genscript. The ribonucleoprotein (RNP) complex was formed with 150 pmol sgRNA and 125 pmol of IDT's Alt-R S.p.Cas9-GFP V3 protein. The RNP was incubated at room temperature for 20 minutes. The GenExact ssDNA donor template was annealed to a DNA oligo to form partial dsDNA structure by combining ssDNA with oligo at a 1:4 molar ratio and incubating at 70° C. for 5 minutes and gradually decreasing the temperature to 4° C. at a rate of 5° C. per 5 minutes. 1 μg (3.37 pmol) of annealed template was used in the nucleofection along with 120 μmol of IDT Cas9 Electroporation Enhancer. The complexed RNP was combined with the annealed donor according to manufacturers' protocols. COS-7 cells were nucleofected using the Lonza 4D-Nucleofector platform and recovered in media containing IDT's HDR enhancer for 24 hours before isolating single cell clones via FACS sorting. Clones were screened with PCR and nanopore sequencing to confirm the presence of a correct heterozygous insertion and intact wild-type allele.

Vimentin-HaloTag® protein COS-7, RPE-1, HEK293T, NIH/3T3, MEF, and NHDF stable lines were generated by lentiviral transduction. In brief, parent cells were seeded in 6 well dishes and incubated with different doses (0.5-2 μL) of high titer (>108 TU/ml) lentivirus for 24 hours. Cells were then washed twice in fresh media, and expression levels/cell viability was monitored over several days before selecting polyclonal lines with intermediate expression levels.

### Example 4: Immunocytochemistry

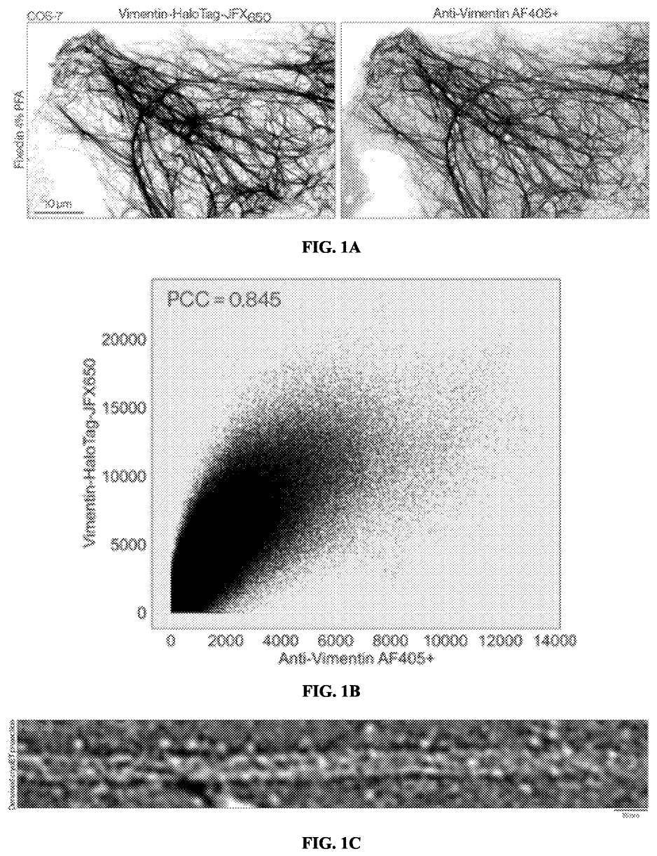

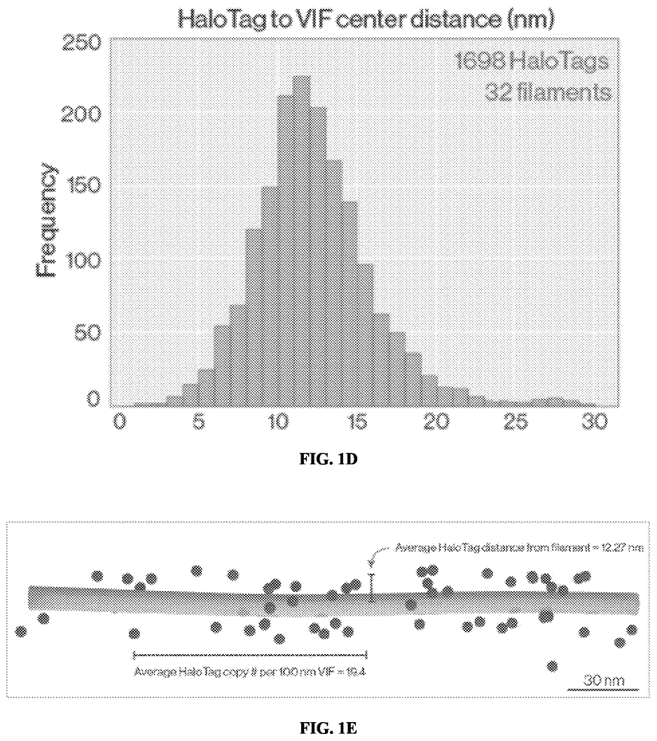

Polyclonal chicken anti-Vimentin (Encor biotech, CPCA-Vim) and Goat anti-chicken IgY Alexa 405-plus (ThermoFisher, A48260) were used for staining vimentin (FIG. 1A). Fixation was performed with 4% paraformaldehyde in PBS (electron microscopy sciences), followed by 3× washes in PBS, permeabilization in 0.25% Triton X-100 for 1 h, and incubation overnight with primary antibodies in 0.1% TritonX100 in PBS. Samples were washed 3× in PBS, and incubated for one hour with secondary antibodies in 0.1% Triton X-100 before imaging in PBS.

### Example 5: Dye Labeling and HTL Screen

Stable vimentin-HaloTag MEFs were seeded in 12-well glass-bottom plates (CellViz). At ~75% confluency, cells were incubated with HaloTag® ligand (250 nM, 0.5-1 h) in complete medium. Cells were then washed 3× in complete DMEM and transferred to a temperature and CO2 controlled stage for widefield epifluorescence microscopy. Time-lapse movies were acquired at one frame per second with a 63×/1.4NA objective lens and 1.6 optovar. At each frame, the sample was irradiated for 100 ms using either 488 nm, 561 nm, or 640 nm laser lines (see power measurements and irradiance/energy calculations below). Timelapse movies were acquired for 600 frames at 1 Hz, but were terminated earlier if all cells in the field of view had undergone IF fragmentation. Movies were then blinded and manually analyzed to determine the latency (number of seconds/irradiation cycles) to initial VIF fragmentation.

### Example 6: Imaging Systems

Airyscan confocal microscopy. Airyscan microscopy was performed on either a Zeiss 880 or Zeiss 980 using an Axio Observer Z1 inverted microscope. Samples were illuminated with 488 nm/561 nm/633 nm laser lines on the Zeiss880 and 488 nm/561 nm/639 nm laser lines on the Zeiss 980. In both cases, samples were imaged with a Plan-Apochromat 63×/1.4 Oil DIC M27 objective lens. Samples imaged on the 980 were acquired using Airyscan 2.0 SR mode and automatically processed in Zen Blue (Zeiss).

Confocal laser power and irradiance measurements. Laser power at the sample plane was measured using a Newport 1936-R power meter coupled with a calibrated silicon photodiode sensor (Newport 818 series). Because the microscope configuration was inverted, the sensor was placed directly at the sample position above the objective lens. To calculate irradiance, the effective beam area was determined by performing a spot bleach experiment using the 561 nm laser line at 100% power on fixed Alexa Fluor 555-immunostained samples. Images were inverted, and a Gaussian function was fit to the intensity profile obtained from a one-pixel-wide line scan across the bleached region. The full width at half maximum (FWHM) calculated from the fit FWHM=2√(2 In 2)·σ) was 488.1 nm, corresponding to a beam radius of approximately 244.05 nm, consistent with the theoretical diffraction-limited spot size.

Laser power measurements were taken at various laser power percentage settings in bidirectional scanning mode, the mode used during imaging experiments. Due to AOTF-mediated laser blanking during scanner retrace periods, these measurements represent time-averaged power, slightly underestimating continuous laser output. To quantify this difference, power was additionally measured using “spot mode,” with the laser continuously unblanked and focused at the sample plane for five seconds. Bidirectional scanning mode measurements were found to be 85.5%±1.0% (mean±SD) of those obtained in spot mode. For example, at 1% laser power, bidirectional scanning yielded 3.16 μW, while spot mode yielded 3.72 μW.

Measured laser power values were used to generate a calibration curve through least-squares linear regression, focusing on the 0.01%-1% power range, which encompassed the settings most frequently used during imaging experiments. This calibration allowed interpolation of laser power across all experimental settings. Irradiance (W/cm²) was calculated by dividing measured or interpolated power by the effective beam area determined from the bleaching experiments. The energy dose per pixel (J/cm²) was subsequently determined by multiplying irradiance by the pixel dwell time. Similar procedures were performed to measure laser power.

### Example 7: Widefield Microscopy

Widefield epifluorescence, total internal reflection fluorescence (TIRF) microscopy, and Lattice SIM were performed on a Zeiss Elyra 7 using an AxioObserver inverted microscope. Samples were illuminated with 500 mW 640 nm, 561 nm, and 488 nm laser lines using a Plan-Apochromat 63×/1.4 Oil DIC M27 objective lens and 1.6× optovar. Emission was collected with dual EM-CCD cameras.

### Example 8: Widefield Laser Power and Irradiance Measurements

Widefield laser power measurements were performed using a Thorlabs PM100D power meter with an S170C microscope slide-format photodiode sensor. All measurements were performed in Laser WF mode using the same imaging configuration as in experiments, including the 63×/1.4 NA objective lens and 1.6× optovar. To determine the effective beam area, a photobleaching experiment was performed using the 561 nm laser at 100% power on a fixed sample immunostained with Alexa Fluor 555. A 3×3 stitched image was acquired after bleaching to visualize the full extent of the bleached region. The bleached area was segmented and quantified from the stitched image to calculate the projected beam area of 13273 μm² at the sample plane.

Laser power was measured at 0.2%, 2%, and 20% settings for each of the three 500 mW laser lines (488 nm, 561 nm, and 642 nm). For the 488 nm line, measured power values were 0.58 mW (0.2%), 2.9 mW (2%), and 20 mW (20%), corresponding to irradiances of 4.4 W/cm², 21.8 W/cm², and 150.7 W/cm², respectively. For the 561 nm line, measured power values were 0.5 mW (0.2%), 2.5 mW (2%), and 29 mW (20%), corresponding to irradiances of 3.8 W/cm², 18.8 W/cm², and 218.5 W/cm², respectively. For the 642 nm line, measured power values were 0.5 mW (0.2%), 2.7 mW (2%), and 20 mW (20%), corresponding to irradiances of 3.8 W/cm², 20.3 W/cm², and 150.7 W/cm², respectively.

### Example 9: Cryo-ET of Detergent-Treated MEFs

MEFs expressing Vimentin-HaloTag® protein were cultured in DMEM (Sigma-Aldrich, D5671), supplemented with 10% FBS (Sigma-Aldrich, F7524), 2 mM 1-glutamine (Sigma-Aldrich, G7513) and 100 μg ml-1 penicillin-streptomycin (Sigma-Aldrich, P0781), at 37° C. and 5% CO₂ in a humidified incubator.

MEFs expressing vimentin-HaloTag® protein were cultured to ~80% confluency on glow-discharged holey carbon EM grids (R2/2, Au 200 mesh; Quantifoil). MEF grids were washed in 37° C. PBS, 2 mM MgCl₂ for 5 s before being transferred for 30 s into 37° C. PBS, 0.1% Triton X-100, 10 mM MgCl₂, 600 mM KCl, complete protease inhibitors. Grids were washed in PBS, 2 mM MgCl₂ for 10 s and then incubated for 30 minutes in PBS, 2 mM MgCl₂, 2.5 units/μl benzonase, 40 ng/μl DNase I at room temperature. After the enzymatic treatment, the grids were washed for 5 s in PBS, 2 mM MgCl₂ and 3 μl of 10 nm fiducial gold markers (Aurion) was applied. The grids were immediately blotted for 5 s from the reverse side and plunge frozen in liquid ethane using a manual plunge freezer.

Tilt-series were collected using a 300 keV Titan Krios with a K2 Summit detector and Quantum energy filter set to a slit width of 20 eV. Data acquisition was performed using SerialEM and PACE-tomo in low-dose mode at ×81.000 magnification, pixel size 1.72 Å, over a ±60° dose symmetric tilt range with 3° increments, −4 μm defocus, and a cumulative dosage of ~150 e-/Å2. Images were drift-corrected using MotionCor2 (Zheng et al. 2017) and aligned using fiducial markers in IMOD. Tomograms were reconstructed and binned 6 times (pixel size 10.8 Å) with weighted back-projections, and a SIRT-like filter was used for enhancing image contrast. HaloTag® proteins were observed as spherical densities with a 3-4 nm diameter, fitting the expected size, and distributed within a 30 nm distance around well-assembled vimentin filaments (diameter ~11 nm). A subset of vimentin filaments and HaloTag® proteins were manually picked with Napari and used to train convolutional neural network models in crYOLO. Vimentin filaments and HaloTag® proteins were predicted using neural network models and manually curated in five tomograms. MATLAB scripts were used to calculate vimentin filament trajectories through tomogram volumes by linear interpolation of vimentin coordinates at filament centers. Vimentin interpolations were used to calculate the distance and distributions of HaloTag® proteins to and along vimentin filaments. Tomogram images and vimentin-HaloTag® protein 3D model were prepared in IMOD and Blender 3D.

### Example 10: Dye Synthesis

Commercial reagents were obtained from reputable suppliers and used as received. All solvents were purchased in septum-sealed bottles stored under an inert atmosphere. All reactions were sealed with septa through which a nitrogen atmosphere was introduced unless otherwise noted. Reactions were conducted in round-bottomed flasks or septum-capped crimp-top vials containing Teflon-coated magnetic stir bars. Heating of reactions was accomplished with a silicon oil bath or an aluminum reaction block on top of a stirring hotplate equipped with an electronic contact thermometer to maintain the indicated temperatures.

Reactions were monitored by thin layer chromatography (TLC) on precoated TLC glass plates (silica gel 60 F₂₅₄, 250 μm thickness) or by LC/MS (Phenomenex Kinetex 2.1 mm×30 mm 2.6 μm C18 column; 5 μL injection; 5-98% MeCN/H₂O, linear gradient, with constant 0.1% v/v HCO₂H additive; 6 min run; 0.5 mL/min flow; ESI; positive ion mode). TLC chromatograms were visualized by UV illumination or developed with p-anisaldehyde, ceric ammonium molybdate, or KMnO₄ stain. Reaction products were purified by flash chromatography on an automated purification system using pre-packed silica gel columns or by preparative HPLC (Phenomenex Gemini NX-C18 30×150 mm 5 μm column). Analytical HPLC analysis was performed with a Phenomenex Gemini NX-C18 4.6×150 mm 5 μm column under the indicated conditions.

NMR spectra were recorded on a 400 MHz spectrometer. ¹H and ¹³C chemical shifts were referenced to TMS or residual solvent peaks. Data for ¹H NMR spectra are reported as follows: chemical shift (8 ppm), multiplicity (s=singlet, d=doublet, t=triplet, q=quartet, dd=doublet of doublets, m=multiplet), coupling constant (Hz), integration. Data for ¹³C NMR spectra are reported by chemical shift (8 ppm) with hydrogen multiplicity (C, CH, CH₂, CH₃) information obtained from DEPT spectra.

### Example 11: JF₆₃₄ (2′,7′-dibromo-JF₆₀₈)

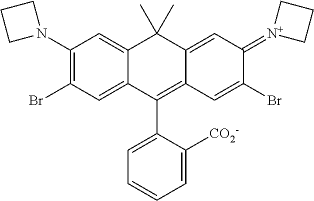

JF₆₀₈ (73 mg, 0.167 mmol) was taken up in CH₂Cl₂ (2 mL) and cooled to 0° C. N-Bromosuccinimide (59.6 mg, 0.335 mmol, 2 eq) in DMF (1 mL) was added dropwise over 5 min, and the reaction was stirred at 0° C. for 15 min. It was subsequently diluted with saturated NaHCO₃ and extracted with EtOAc (2×). The combined organic extracts were dried over anhydrous MgSO₄, filtered, and concentrated in vacuo. Silica gel chromatography (0-50% EtOAc/toluene, linear gradient) afforded 79.3 mg (80%) of the title compound as a pale blue solid. ¹H NMR (CDCl₃, 400 MHz) δ 8.04-8.00 (m, 1H), 7.65 (td, J=7.4, 1.4 Hz, 1H), 7.60 (td, J=7.4, 1.2 Hz, 1H), 7.09-7.05 (m, 1H), 6.67 (s, 2H), 6.63 (s, 2H), 4.18-4.07 (m, 8H), 2.29 (p, J=7.3 Hz, 4H), 1.80 (s, 3H), 1.71 (s, 3H); ¹³C NMR (CDCl₃, 101 MHz) δ 170.3 (C), 154.1 (C), 149.8 (C), 145.5 (C), 135.1 (CH), 133.4 (CH), 129.7 (CH), 126.9 (C), 125.5 (CH), 123.9 (CH), 123.6 (C), 111.6 (CH), 106.9 (C), 86.2 (C), 54.4 (CH₂), 38.4 (C), 35.6 (CH₃), 32.2 (CH₃), 16.8 (CH₂); Analytical HPLC: t_{R}=17.2 min, >99% purity (10-95% MeCN/H₂O, linear gradient, with constant 0.1% v/v TFA additive; 20 min run; 1 mL/min flow; ESI; positive ion mode; detection at 650 nm); MS (ESI) calcd for C₂₉H₂₇Br₂N₂O₂ [M+H]⁺ 595.0414, found 595.2.

### Example 12: JF₆₃₄-HaloTag® Ligand (2′,7′-Dibromo-JF₆₀₈-HaloTag® Ligand)

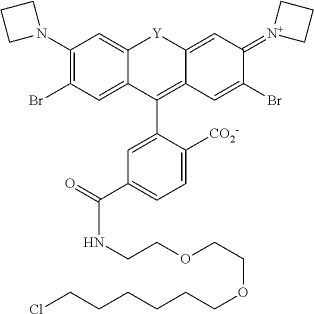

JF₆₀₈-HaloTag® ligand (20 mg, 0.029 mmol) was taken up in CH₂Cl₂ (1 mL) and cooled to 0° C. N-Bromosuccinimide (10.4 mg, 0.058 mmol, 2 eq) in DMF (0.5 mL) was added dropwise over 5 min, and the reaction was stirred at 0° C. for 15 min. It was subsequently diluted with saturated NaHCO₃ and extracted with EtOAc (2×). The combined organic extracts were washed with brine, dried over anhydrous MgSO₄, filtered, and concentrated in vacuo. Purification by reverse phase HPLC (50-95% MeCN/H₂O, linear gradient, with constant 0.1% v/v TFA additive) afforded 10.5 mg (43%) of the title compound as a pale blue solid. ¹H NMR (CDCl₃, 400 MHz) δ 7.99 (dd, J=8.0, 0.7 Hz, 1H), 7.89 (dd, J=8.0, 1.4 Hz, 1H), 7.39 (dd, J=1.4, 0.8 Hz, 1H), 6.77 (t, J=4.6 Hz, 1H), 6.58 (s, 2H), 6.54 (s, 2H), 4.06 (t, J=7.3 Hz, 8H), 3.60-3.52 (m, 6H), 3.50-3.47 (m, 2H), 3.45 (t, J=6.7 Hz, 2H), 3.33 (t, J=6.6 Hz, 2H), 2.23 (p, J=7.3 Hz, 4H), 1.74 (s, 3H), 1.71-1.65 (m, 2H), 1.63 (s, 3H), 1.47-1.42 (m, 2H), 1.39-1.31 (m, 2H), 1.29-1.23 (m, 2H); Analytical HPLC: t_{R}=15.3 min, >99% purity (30-95% MeCN/H₂O, linear gradient, with constant 0.1% v/v TFA additive; 20 min run; 1 mL/min flow; ESI; positive ion mode; detection at 650 nm); MS (ESI) calcd for C₄₀H₄₇Br₂ClN₃O₅ [M+H]⁺ 844.1546, found 844.3.

### Example 13: Mass Spectrometry

Solvents and Chemicals: Water (Optima, W6-4), acetonitrile (ACN, Optima, A9554), methanol (Optima, A454SK-4), formic acid (Pierce, PI28905), Acetone (A949-1, Fisher) RapiGest SF Surfactant (Waters, 186001861). Dithiothreitol (DTT, R0861, Thermo scientific), Trifluoroacetic acid (TFA, 302031-100ML), Iodoacetamide (IAA, 407710) and ammonium bicarbonate (40867) were purchased from Sigma Aldrich. Trypsin (V5113) was purchased from Promega.

Protein Digestion and Desalting: The concentration of the proteins from each cell lysate was assessed through BCA assay. The same amount of proteins from each sample were then precipitated with cold acetone at 1:4 (v:v) ratio, −20° C. overnight. Protein pellets were centrifuged and washed with cold acetone the next day and the residue acetone was air dried. The precipitated protein pellets were re-dissolved using 0.5% RapiGest according to the manufacturer's protocol. Afterwards, proteins were reduced with 5 mM DTT (60° C., 30 min) and alkylated using 11 mM IAA at ambient temperature in the dark for 30 minutes. Trypsin was added at a protein-enzyme ratio of 50:1 for overnight digestion at 37° C. The digestion was quenched by adding 10% TFA to PH~1. The digest was further incubated at 37° C. for 45 min to degrade the RapiGest. The solutions were then centrifuged at 14 000 rpm for 10 min, and the supernatant peptides were collected. Desalting of the peptides was performed using C18 ZipTip (Millipore, ZTC18M096) and the eluents were dried using SpeedVac (Thermo Scientific). The samples were stored at −80° C. before being re-suspended in 0.1% formic acid for LC-MS/MS analysis.

LC-MS/MS Analysis: LC separation was performed on a Vanquish Neo System (Thermo Scientific) with an IonOptik Aurora Ultimate C18 column (25 cm length×75 μm inner diameter×1.7 μm particle size) at 50° C. and 300 nL/min flow rate. Mobile phase A consisted of 0.1% formic acid in water. Mobile phase B consisted of 0.1% formic acid in 80% ACN. Eluting peptides were ionized by electrospray ionization and then analyzed by an Orbitrap Ascend Tribrid mass spectrometer (tune version 4.1.4244, Thermo Scientific). Ion transfer tube temperature was set to 275° C. Source positive ion voltage was set to 1850v. For single-shot proteomics with data dependent acquisition (DDA), the MS1 scan resolution was set to 120,000 (at m/z 200). MS1 scan range was 350-1500 m/z, AGC target was 250%, maximum injection time mode was set to auto. Precursors were isolated through quadrupole with an isolation width of 1.2 (m/z). Precursors were fragmented by HCD at an NCE of 26%. MS2 scans were acquired by ion trap using rapid rate.

Data Processing: Raw data was directly processed using Thermo Proteome Discoverer (3.0.0.757) against the Homo sapiens (UP000005640) proteome fasta file with the Halo tag attached to the vimentin sequence. A maximum of one missed cleavage was allowed and cysteine carbamidomethylation (+57.0215 Da) as the fixed modification. Oxidation (M), Phosphorylation (S, T, Y) and acetylation at protein N-terminus were selected as variable modification. Percolator node was used, and the target FDR (strict) was set to 0.01. 10 ppm was used as the precursor mass tolerance and 0.2 Da for the fragment ion tolerance window. Label-free quantification information of the peptides and proteins was processed with sample runs normalized according to the total peptide amount.

### Example 14: Image Processing and Analysis

Local image coherency, energy, and orientation were measured with the ImageJ plugin OrientationJ using cubic spline 2px gradients. To measure local coherence changes over time, coherency maps were threshold by 50% energy and mean values were plotted over time. Coefficient of Variation (CV) and normalized radial vimentin intensity were analyzed using custom MATLAB scripts. Normalized CV change computes the fractional change in CV at each frame relative to the first frame's CV. This metric allows for easier comparison between CV changes in images with different baseline signal to noise ratios and was implemented in Python. For wide field epifluorescence movies, bleach correction with exponential fit was performed in ImageJ for dyes that displayed appreciable bleaching over the imaging window. For presentation, a subset of movies was filtered using the unsharp mask filter in Fiji using a radius of 5 px and mask weight of 0.6 pt before using a three-frame sum intensity projection, increasing VIF contrast but reducing temporal resolution. Existing MATLAB scripts were converted to Python.

Single particle tracking (SPT) data was performed in ImageJ using the TrackMate plugin. For SPT of VIF fragments, images were convolved with a Gaussian blurring filter with a kernel size of 2 px. Particle localization was performed using the Hessian detector algorithm, and the simple LAP Tracker was used to generate trajectories with the following conditions: 500 nm maximum gap closing distance, no frame gaps, and 500 nm frame-to-frame linking distance. For SPT of lipid droplets, the Laplacian of Gaussian detector with an estimated object diameter of 0.3 μm was used to identify LDs, and the simple LAP tracker was used with 200 nm linking max distance, 500 nm gap-closing max distance, and gap-closing max frame gap of 2. For VIF fragment trajectories, mean-squared displacements (MSDs) were calculated in Mathematica 14.0 and were piecewise fit to MSD(t)=Dt^{a} for effective diffusion coefficient D, time t, and time dependence a. For LDs, α values were obtained in python by fitting a linear model to the log-transformed MSD data over the 2nd through 18th lag time increments, with slope a. MSD, net displacement, and trajectories were plotted in python using the Seaborn library.

Fluorescence Recovery After Photobleaching (FRAP) measurements were performed using ImageJ by selecting ROIs around VIF fragments and quantifying mean fluorescence intensity over time using the Z-Axis Profile function. The recovery curve of mean intensity I(t) was fit using the exponential recovery function: I(t)=A(1−e^{(-t/τ)})+B, where A represents amplitude of fluorescence recovery, t is characteristic recovery time, and B is baseline offset. Mobile fraction was calculated by normalizing amplitude A to the mean fluorescence intensity prior to photobleaching, and immobile fraction was determined as (1-mobile fraction).

### Example 15: Code Availability

The python and imagej macro language scripts used for calculating and plotting coefficient of variation, normalized radial position, local image coherency, and MSD for VIF fragments and lipid droplets are available at github.com/andmoo91/FilaBusterScripts.

### Example 16: Vimentin-HaloTag® Protein Incorporates into Native Vimentin IFs and Meets Spatial Requirements for Targeted Oxidative Disassembly

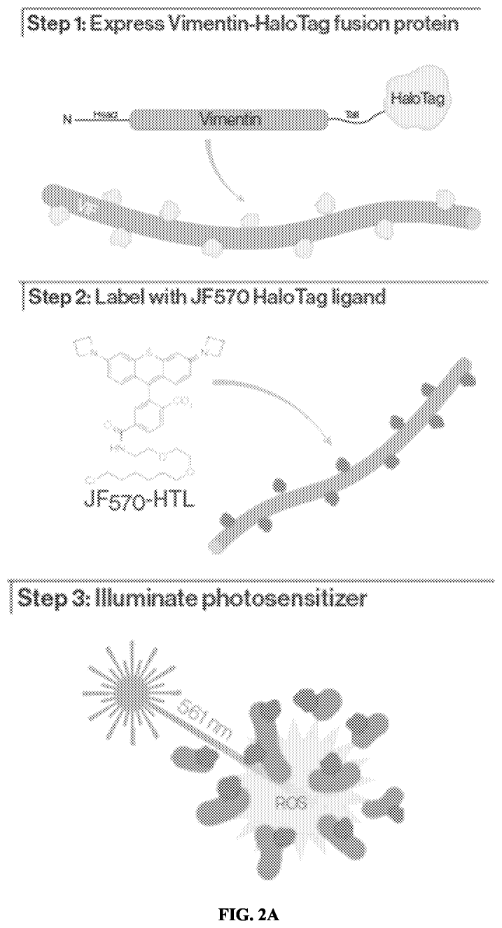

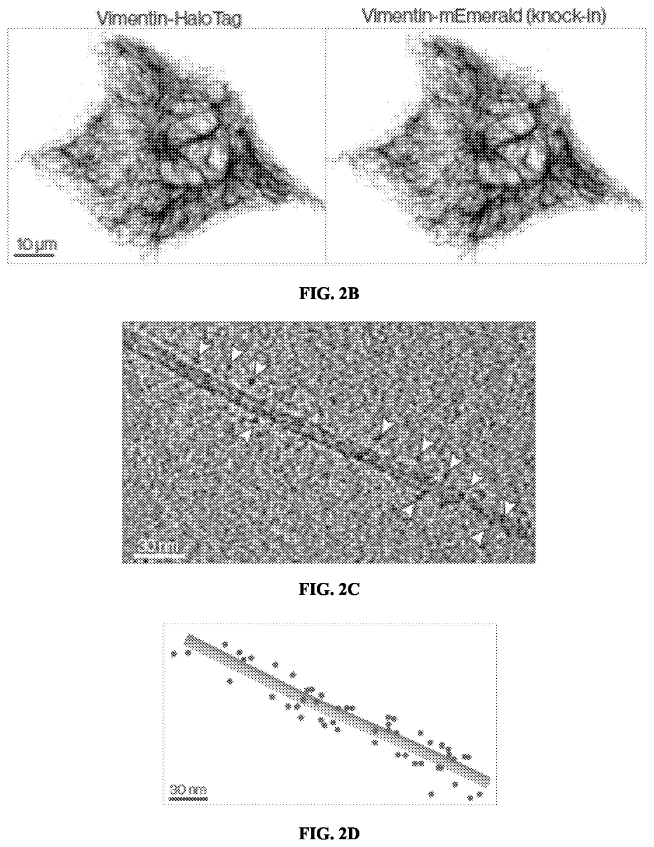

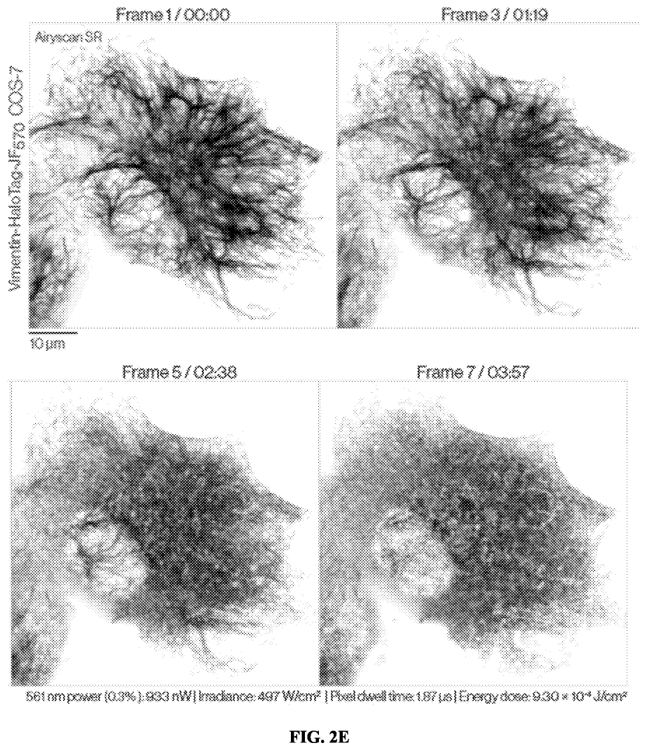

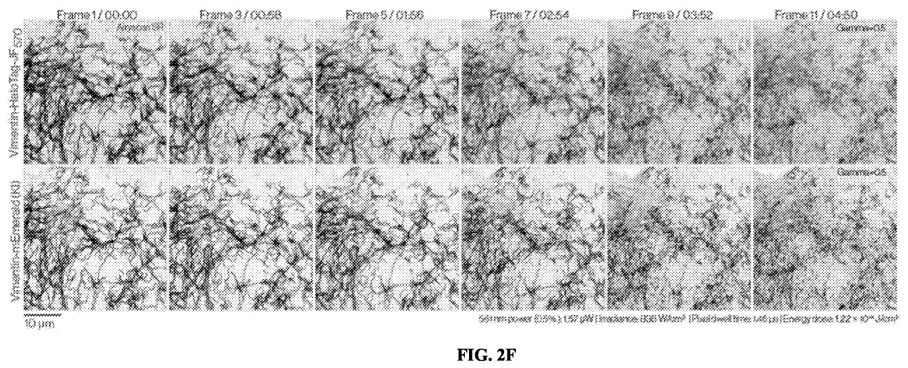

To prototype the FilaBuster approach, vimentin IFs were initially selected as a proof-of-concept model system. Vimentin is among the best-characterized IF proteins, forms simple homopolymers, and its complete filament structure has recently been resolved, making it an ideal starting point for validating and refining the FilaBuster strategy before extension to other IF systems. A fusion protein linking HaloTag7 to the C-terminal tail of human vimentin was designed. To establish its specificity and proper integration into the native vimentin IF (VIF) network, vimentin-HaloTag® protein was first stably expressed in wild-type COS-7 cells. After fixation and immunostaining, Airyscan confocal microscopy revealed extensive colocalization between the HaloTag® fusion and immunolabeled filaments, confirming proper incorporation into endogenous VIFs (FIG. 1A-1B). To further validate native VIF integration in live cells, the construct was expressed in vimentin-mEmerald knock-in COS-7 cells, where complete overlap between vimentin-HaloTag® protein and endogenously tagged vimentin was observed (FIG. 2B).

Fluorescence microscopy confirmed the specificity of the fusion protein; however, to determine whether its incorporation affected the structural integrity of individual VIFs, cryo-electron tomography (cryo-ET) was applied to permeabilized, plunge-frozen mouse embryonic fibroblasts (MEFs) stably expressing vimentin-HaloTag® protein. Tomogram analysis revealed intact vimentin filaments, approximately 11 nm in diameter, surrounded by 3-4 nm spherical densities, consistent with the dimensions of HaloTag® protein (FIG. 1C, FIG. 2C-2D). HaloTag® proteins were positioned at a mean distance of 12.27±3.75 nm from the filament center (FIG. 1D), with an average of 1.94±0.43 HaloTag® proteins per 10 nm of filament length. Given that each 10 nm segment of vimentin contains 12-14 tail domains, this corresponds to a mean HaloTag® protein occupancy of ~15% (FIG. 1E). Together, these findings indicate that the vimentin-HaloTag® protein fusion integrates into endogenous VIFs without compromising filament architecture. Furthermore, the HaloTag® proteins are positioned in close proximity to the filament surface—within a range that permits effective interaction with short-lived ROS generated by photosensitizing ligands. Finally, the uniform distribution of HaloTag® proteins along the filament ensures extensive coverage, enhancing the likelihood of comprehensive and efficient filament disassembly upon photosensitizer labeling and subsequent irradiation.

### Example 17: Controlled Disassembly of VIFs by Activation of a HaloTag-Targeted Photosensitizer

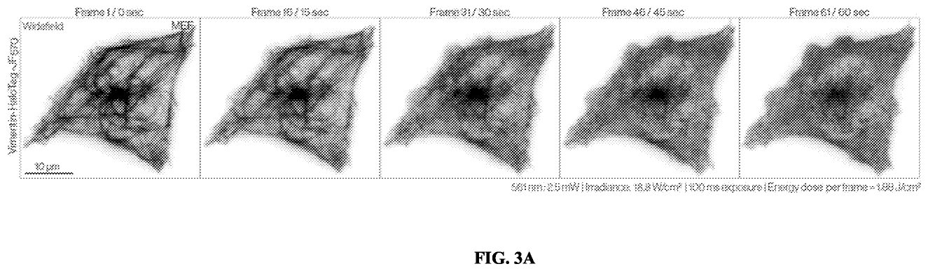

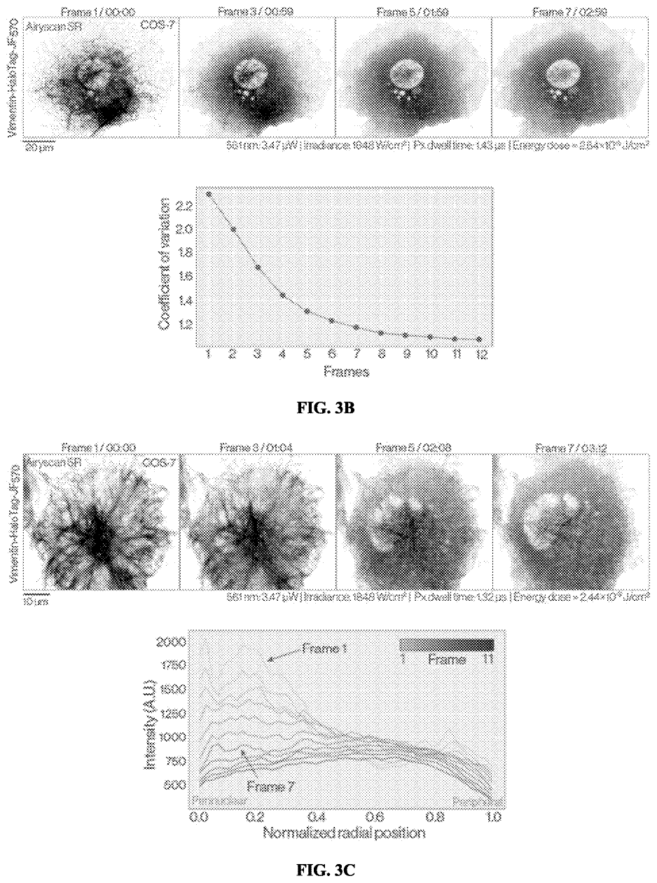

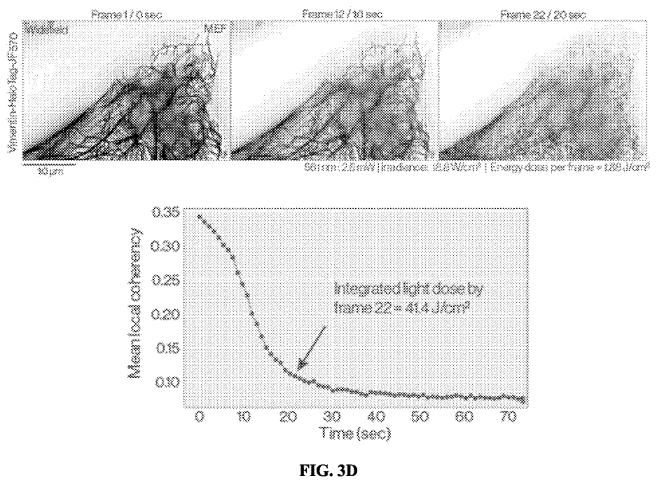

Having established that the probe effectively integrates into endogenous filaments, its functional utility for light-mediated VIF disassembly was next assessed. To this end, vimentin-HaloTag® protein-expressing cells were labeled with the photosensitizer JF₅₇₀-HTL (250 nM, 30 min), and VIF disruption was attempted using 561 nm illumination. To prevent premature activation, cells were initially monitored under transmitted light and appeared healthy with normal morphology. Airyscan (FIG. 2E) or widefield (FIG. 3A) time-lapse movies were then acquired using low-power 561 nm laser illumination (see methods/figure legends for details). In the initial frames, VIFs displayed normal morphology, forming an extensive filamentous network extending throughout the cytoplasm. However, beginning at approximately five illumination cycles, progressive and widespread VIF fragmentation was observed, accompanied by a steady rise in diffuse cytoplasmic fluorescence consistent with increasing levels of soluble vimentin subunits (FIG. 2E, FIG. 3A).

To quantitatively assess VIF disassembly, three distinct metrics that reflect filament network integrity were evaluated. First, the coefficient of variation—a measure of fluorescence intensity heterogeneity—decreased over time, consistent with the disappearance of dense filament bundles and a shift toward a more uniform, diffuse signal (FIG. 3B). Second, the radial intensity profile, which reflects the spatial distribution of VIFs from nucleus to periphery, progressively flattened, consistent with the dispersal of VIF fragments away from the perinuclear region (FIG. 3C). Finally, local image coherency, a measure of directional filament alignment, dropped sharply, reflecting the breakdown of elongated filaments into isotropic fragments (FIG. 3D).

To confirm that the approach disassembles native vimentin filaments, the experiment was repeated in vimentin-mEmerald knock-in COS-7 cells co-expressing vimentin-HaloTag-JF₅₇₀. In these cells, concurrent fragmentation of both HaloTag- and mEmerald-labeled VIFs was observed, confirming that light-induced disruption extends to the endogenous filament network (FIG. 2F).

### Example 18: Photochemical Disassembly of Native VIFs Via Nanobody-HaloTag® Protein Fusions

It was found that the vimentin-HaloTag® protein fusion seamlessly integrates into native VIFs, providing an effective means of stably anchoring photosensitizer ligands at the filament surface. However, introduction of a tagged IF subunit requires careful validation to ensure that the fusion does not interfere with normal filament dynamics. Moreover, ectopic expression of IF subunits—even untagged—can increase the density of the IF network and meaningfully alter its mechanical properties. While these concerns can be mitigated with careful controls and thoughtful experimental design, genetic tagging of IF subunits may not always be feasible or desirable.

An attractive alternative to direct subunit tagging is the use of live cell-expressible nanobodies directed against IF proteins. Comprising a conventional fluorescent protein fused to the variable domain (VHH) of an alpaca heavy-chain antibody, fluorescent nanobodies, or chromobodies, have been developed to label multiple IF subtypes, including keratin 8, lamin, and vimentin. It was hypothesized that a nanobody-HaloTag® protein fusion could be used to direct photosensitizer ligands to native IFs in live cells, effectively circumventing concerns about direct subunit tagging or overexpression.

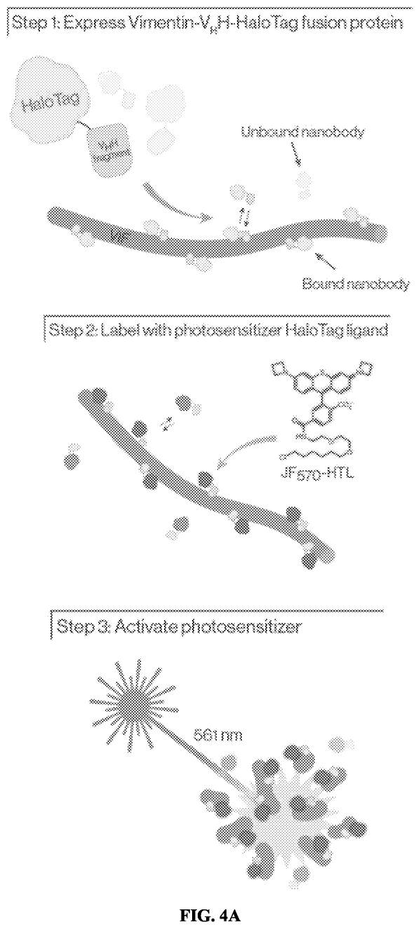

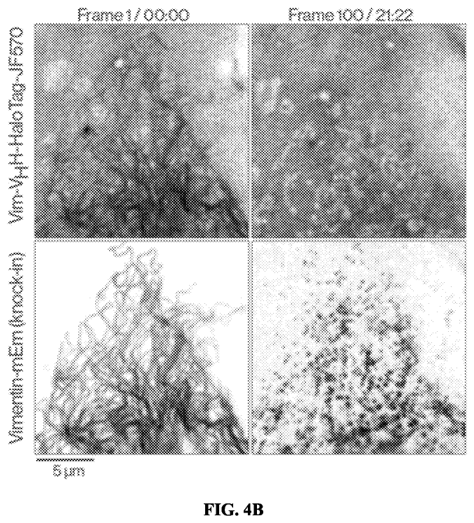

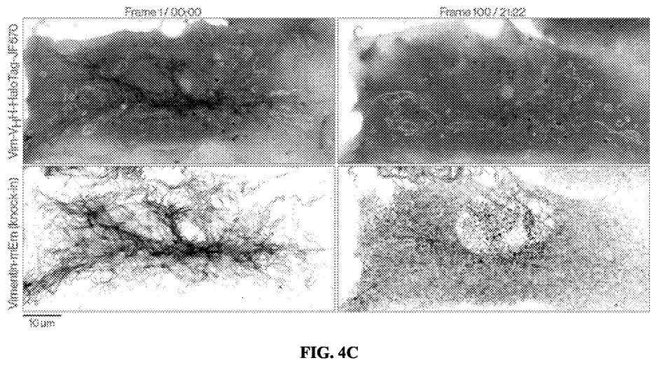

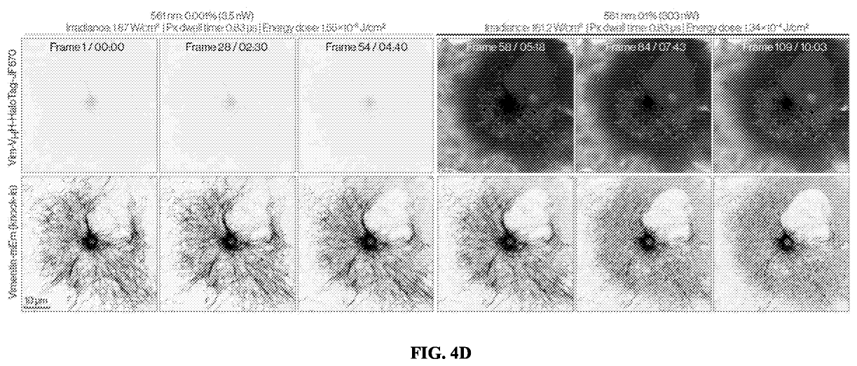

To explore the viability of this approach, a construct linking HaloTag® protein to the C-terminus of a well-characterized vimentin nanobody was generated, and the fusion (vimentin-VHH-HaloTag) was expressed in vimentin-mEmerald knock-in COS-7 cells (FIG. 4A). Fluorescence microscopy confirmed specific binding and localization of the nanobody fusion to the native vimentin network without altering its organization. Labeling vimentin-VHH-HaloTag-expressing cells with JF₅₇₀-HTL followed by 561 nm illumination induced rapid disintegration of endogenous vimentin filaments, with comparable efficiency to that observed with vimentin-HaloTag-JF570 (FIG. 4B-4D). This nanobody-based targeting strategy thus significantly broadens the scope and applicability of FilaBuster, enabling targeted, photochemical disassembly of VIFs without the requirement for direct vimentin tagging. Given the availability of validated nanobodies against other IF proteins, this strategy should be readily adaptable to target additional filament classes.

### Example 19: Expanding FilaBuster with a Red-Shifted, Fluorogenic HaloTag® Ligand Photosensitizer for Controlled VIF Disassembly

Building upon the successful induction of rapid VIF disassembly with JF₅₇₀, the utility of FilaBuster was expanded by developing a red-shifted HaloTag® photosensitizer ligand that could be activated using far-red lasers common on standard fluorescence microscopes. Such a ligand would facilitate deeper tissue penetration due to the longer excitation wavelength and enable multiplexed imaging with conventional blue- and green-excited fluorophores, further enhancing the versatility of the approach.

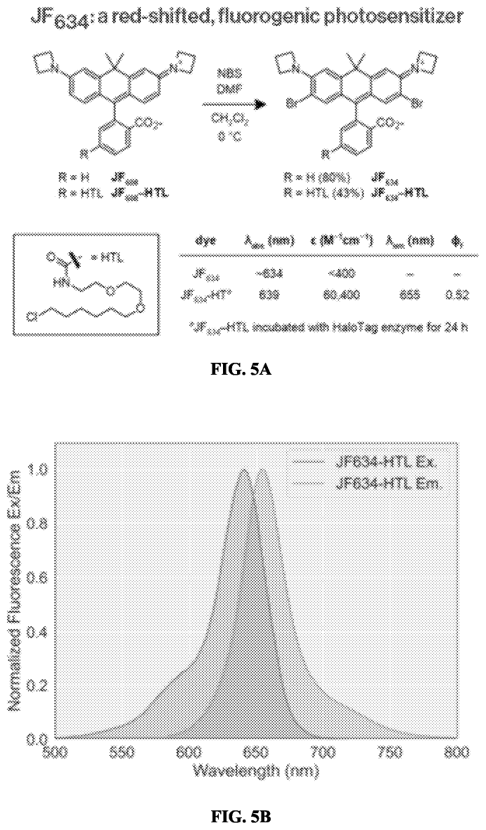

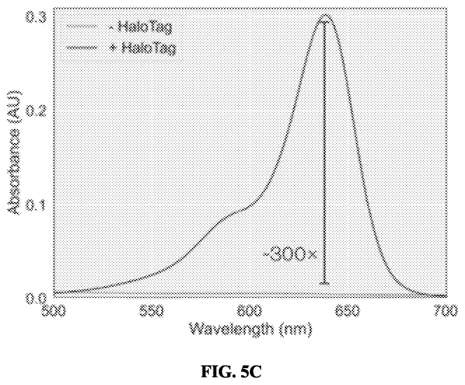

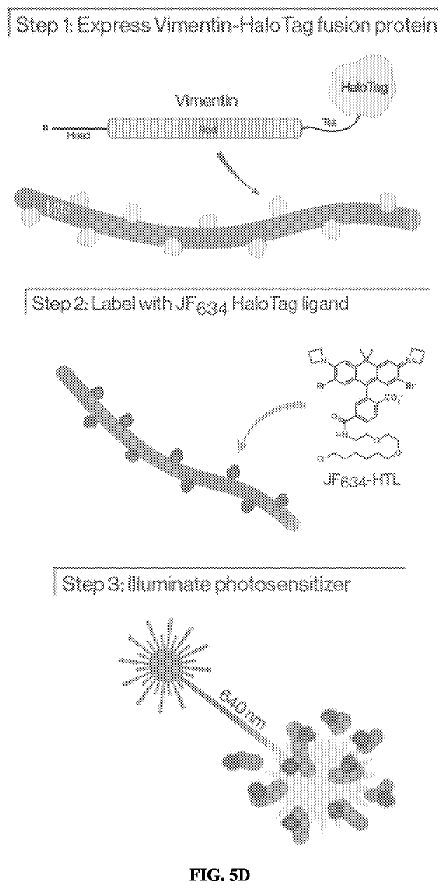

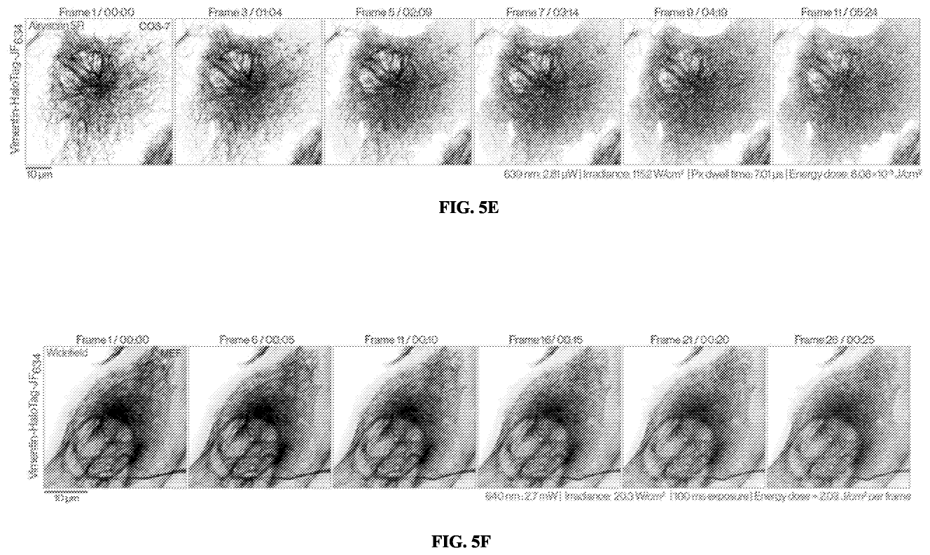

To this end, JF₆₃₄ was synthesized as a derivative of JF₆₀₈ modified with bromine substituents to enhance intersystem crossing efficiency via the heavy-atom effect (FIG. 5A). To allow covalent labeling of HaloTag® fusion proteins, JF₆₃₄ was further functionalized with a chloroalkane linker, generating JF₆₃₄-HTL. Spectroscopic characterization of JF₆₃₄-HTL in the presence of HaloTag® protein revealed a peak absorbance at 639 nm as well as a ~300-fold increase in absorbance compared to the unbound ligand (FIG. 5A-5C).

JF₆₃₄-HTL was next evaluated in live cells stably expressing vimentin-HaloTag® protein (FIG. 5D). The dye exhibited efficient cellular uptake and selective labeling of the filament network, with no detectable cytotoxicity or vimentin cytoskeletal artifacts following prolonged incubation in the dark. Upon illumination with 639 nm (Airyscan) or 640 nm (widefield) light, vimentin-HaloTag-JF₆₃₄ triggered robust and rapid VIF disassembly in both COS-7 and MEFs (FIG. 5E-5F), with disassembly kinetics comparable to, and in some cases faster than, those observed with JF₅₇₀. Together, these results establish JF₆₃₄-HTL as an effective, red-shifted alternative to JF₅₇₀.

### Example 20: Dose-Dependent Control of VIF Fragmentation Using Conventional HaloTag® Ligands

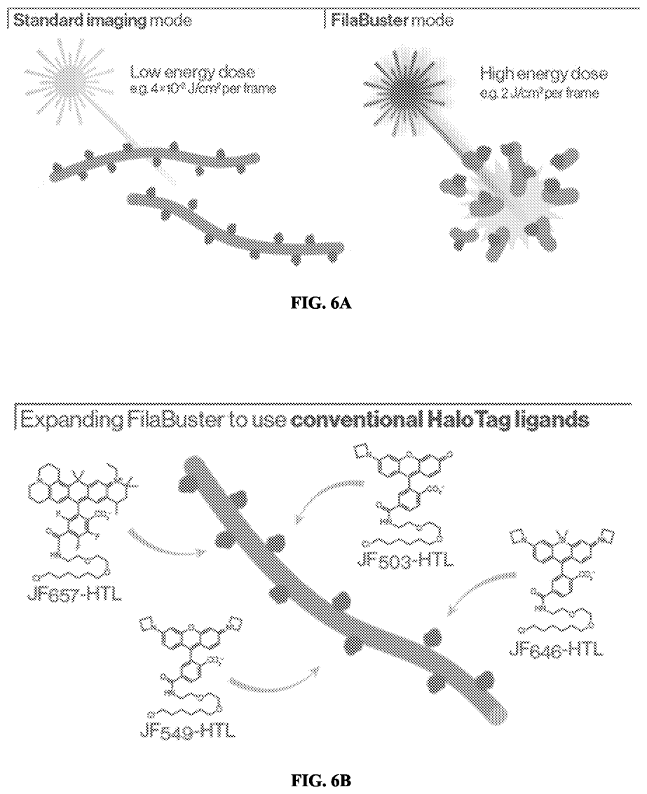

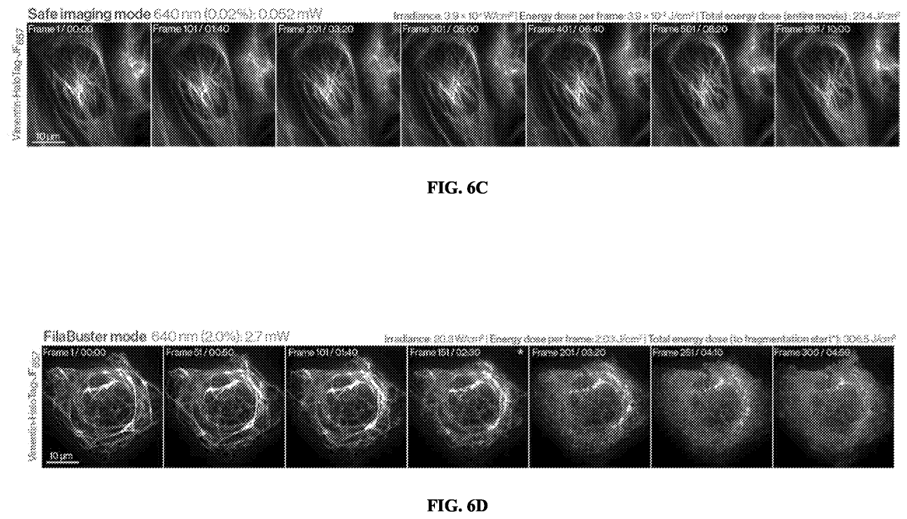

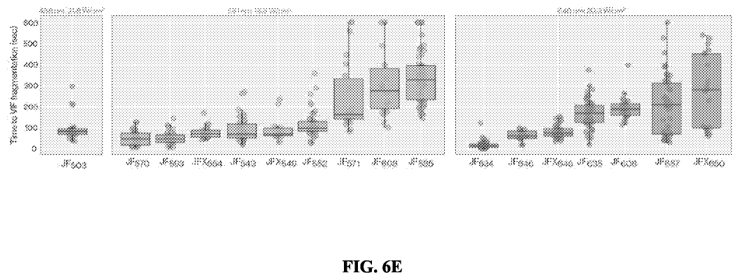

Although the two validated vimentin-targeted photosensitizers induce rapid VIF fragmentation under low-light minimal illumination conditions, their extreme sensitivity to excitation light precludes extended imaging of native VIF dynamics prior to targeted disruption. To overcome this limitation, dyes capable of operating in two distinct modes were identified: a low power “standard imaging” mode for visualizing intact IFs, and a high-power “FilaBuster” mode for inducing filament fragmentation (FIG. 6A).

It was contemplated that conventional rhodamine-based HaloTag® ligands, despite lacking heavy-atom substituents for efficient singlet oxygen production, might exhibit latent photoreactivity under elevated illumination intensities (FIG. 6B). To test this hypothesis, vimentin-HaloTag® protein MEFs were labeled with JF657-HTL, a bright, far-red-excitable carborhodamine, and cells were imaged with a 640 nm laser in either low-power standard mode (irradiance: 0.39 W/cm²) or high-power FilaBuster mode (irradiance: 20.3 W/cm²). In standard mode, VIFs remained intact and morphologically stable over ten minutes or 601 cycles of illumination (FIG. 6C). In contrast, under FilaBuster mode, robust filament disassembly was triggered after approximately 150 frames (FIG. 6D), requiring a cumulative energy dose of 307 J/cm²—over 13-fold greater than the total energy dose over 10 minutes in standard imaging mode (FIG. 6C-6D). These findings demonstrate that conventional HTLs can reliably trigger VIF disassembly when activated at sufficiently high energy levels, despite their lower intrinsic photoreactivity.

### Example 21: Benchmarking Conventional HTLs as FilaBusters

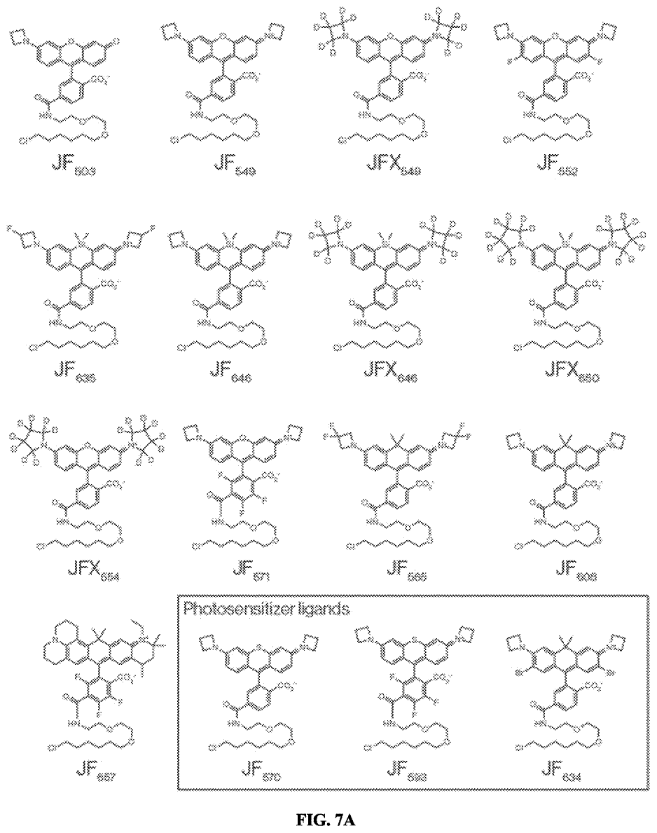

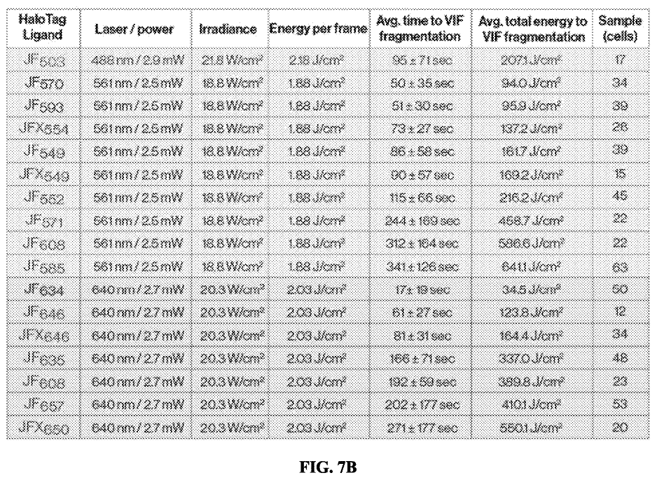

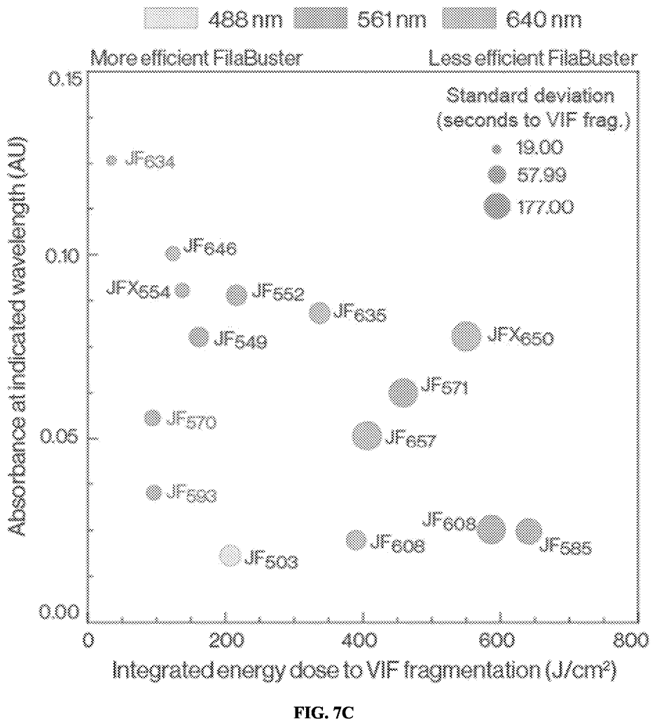

Encouraged by these results, 13 conventional HTLs and 3 photosensitizer HTLs were systematically benchmarked in vimentin-HaloTag® protein MEFs using widefield epifluorescence microscopy under FilaBuster-mode imaging conditions (FIG. 7A). Surprisingly, every ligand successfully induced VIF fragmentation, though with varying efficiency (FIG. 6E, FIG. 7B-7C). As predicted, photosensitizers such as JF634-HTL were the most potent, triggering network collapse within ~17 cycles of 640 nm irradiation. However, several conventional HTLs, including JF549, JF646, and their deuterated JFX analogs, were also highly effective, driving filament disassembly within 60-90 higher-energy illumination cycles. Illumination parameters and corresponding energy thresholds for each ligand are summarized in FIG. 7B. While variation in ligand performance likely reflects differences in intersystem crossing efficiencies, other factors such as dye loading and excitation efficiency at the applied laser line may also contribute (FIG. 7C).

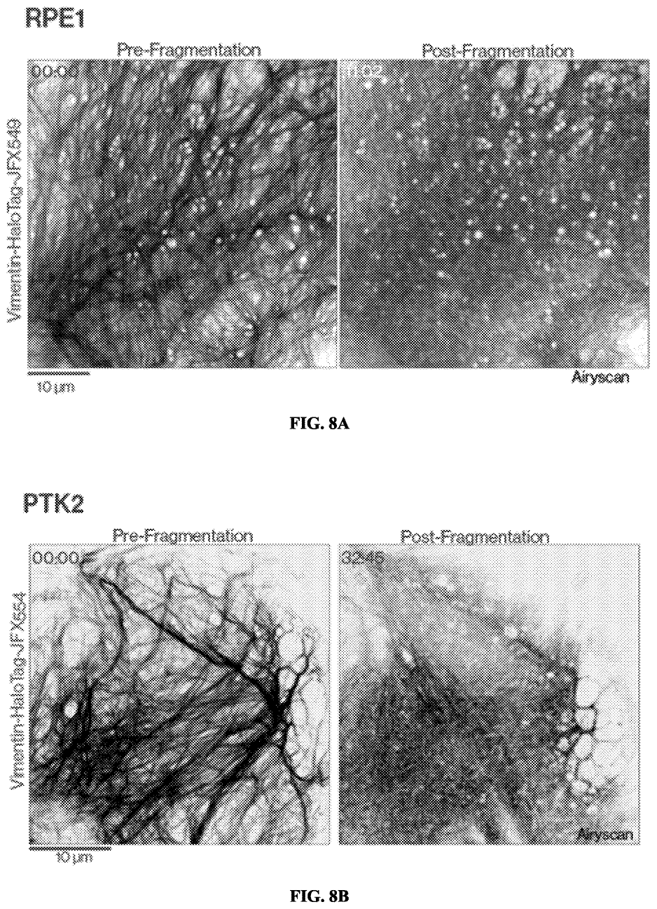

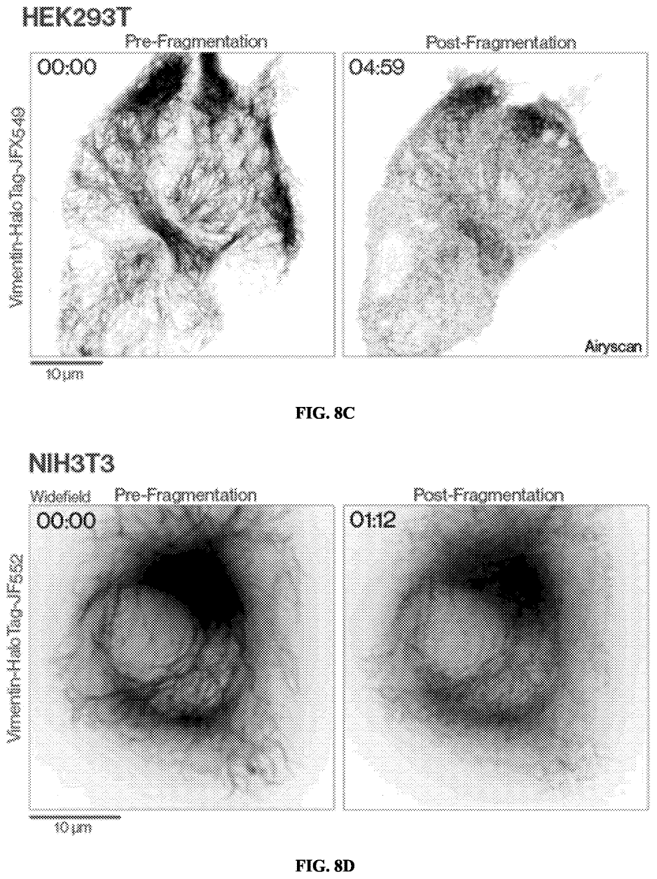

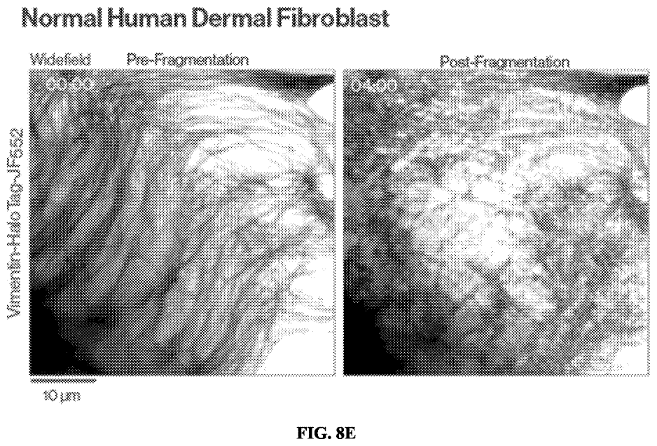

The observation that all tested ligands were capable of driving VIF disassembly under elevated illumination underscores the broad compatibility of rhodamine-based HTLs with the FilaBuster framework. Moreover, similarly efficient VIF disassembly was observed across a wide range of cell types, including epithelial lines, immortalized fibroblasts, and primary human fibroblasts (FIG. 8A-8E), demonstrating that FilaBuster performance generalizes across diverse cellular contexts.

### Example 22: Operational Boundaries of FilaBuster-Induced IF Disassembly

Encouraged by the broad efficacy of conventional and photosensitizer HaloTag® ligands in driving IF disassembly, the mechanistic constraints that govern FilaBuster activity were next defined. Specifically, the degree to which the system depends on spatial targeting, molecular configuration, and the redox sensitivity of the underlying filament network was examined.

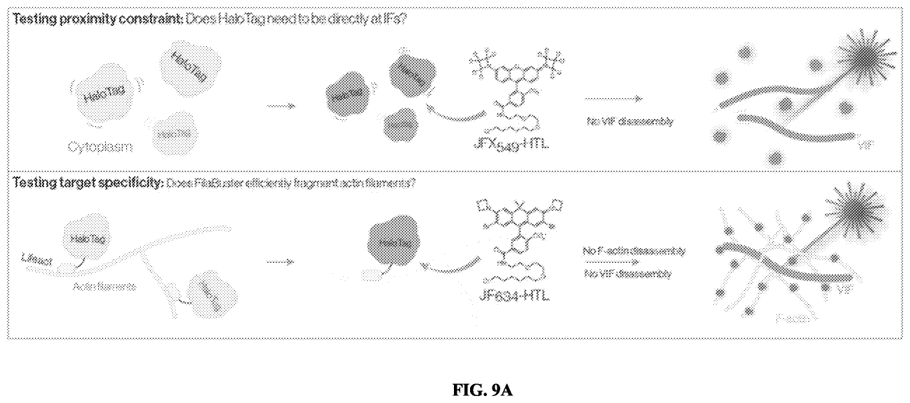

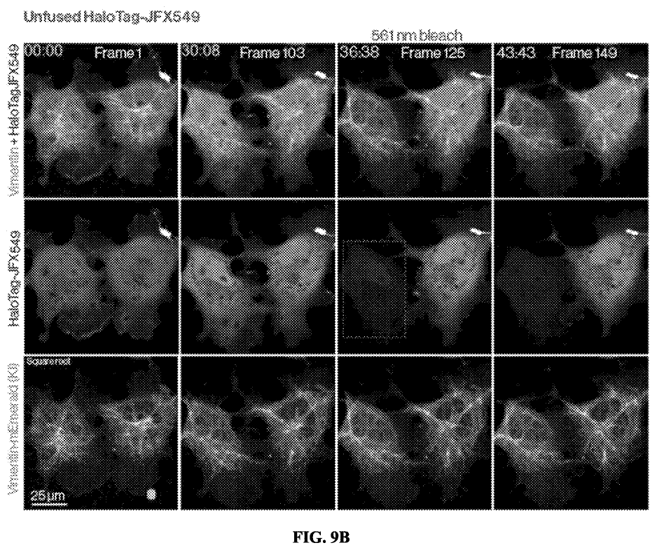

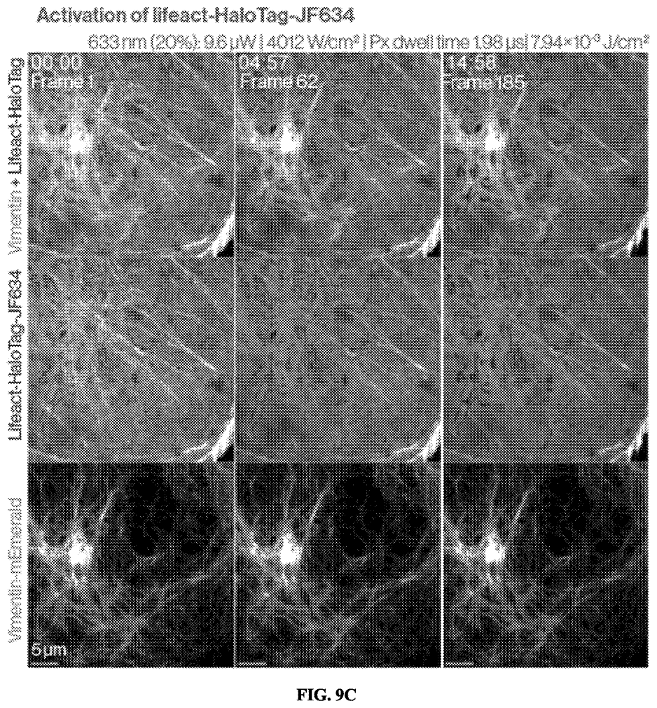

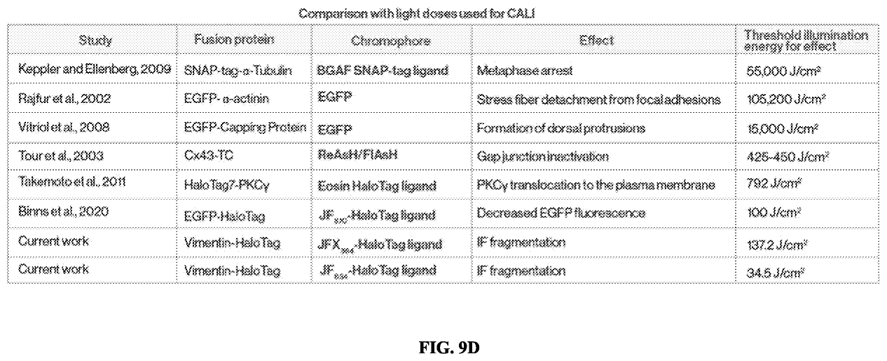

To test the spatial proximity requirements of FilaBuster, it was assessed whether ROS generation must occur directly at the filament surface or whether non-targeted, diffusely distributed photosensitizers throughout the cytoplasm could still drive light-mediated VIF disassembly. As a test of this boundary condition, an unfused, cytosolic HaloTag® protein was expressed, labeled with JFX549-HTL, and cells were subjected to high-intensity 561 nm laser illumination (FIG. 9A-9B). Despite robust ligand photobleaching and energy doses that far exceeded those required to fragment vimentin filaments labeled with the same dye, no VIF disruption was observed (FIG. 9B). These findings reveal a strict spatial requirement for photosensitizer activity: ROS must be generated at the filament surface to induce IF disassembly, consistent with their nanometer-scale diffusion ranges and rapid quenching in the intracellular environment.

It was then assessed whether other intracellular targets are similarly susceptible to localized ROS when delivered via the FilaBuster targeting system. Specifically, it was tested whether directing JF634 to F-actin via a LifeAct-HaloTag® protein fusion would result in actin filament fragmentation under illumination conditions able to induce VIF fragmentation (FIG. 9C). Cells were irradiated with 633 nm light (peak irradiance: 4012 W/cm²), but no obvious actin filament disassembly or off-target effects on VIFs labeled with a conventional vimentin-mEmerald reporter were observed (FIG. 9C). These observations indicate that FilaBuster-induced disruption is not a generalizable effect of local ROS production, but a consequence of targeting a uniquely vulnerable cytoskeletal substrate. Importantly, the integrated energy doses used to drive FilaBuster-mediated VIF disassembly—as little as 34.5 J/cm² in the case of vimentin-HaloTag-JF634—are one to three orders of magnitude lower than those required to induce comparable effects in classical CALI systems, which often exceed 10,000 J/cm² (FIG. 9D).

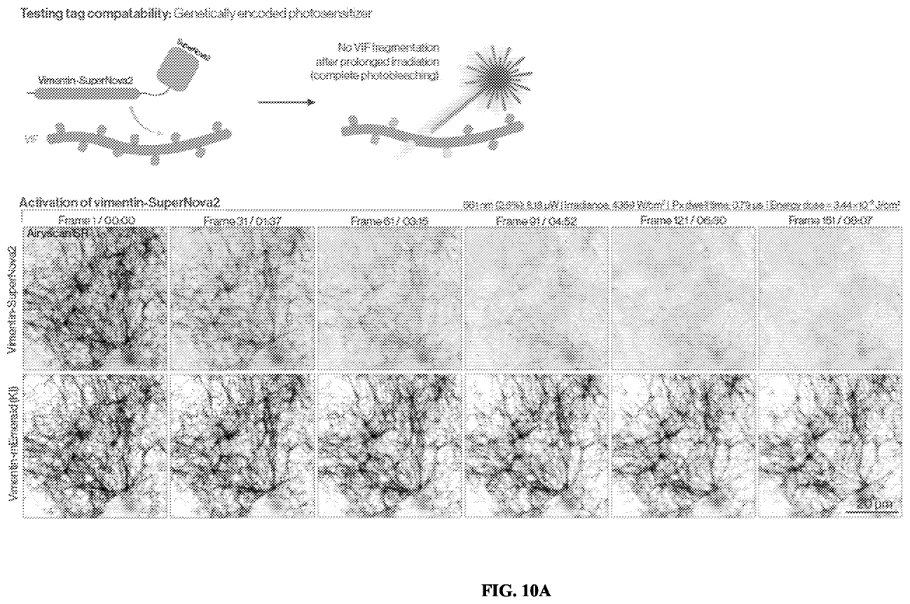

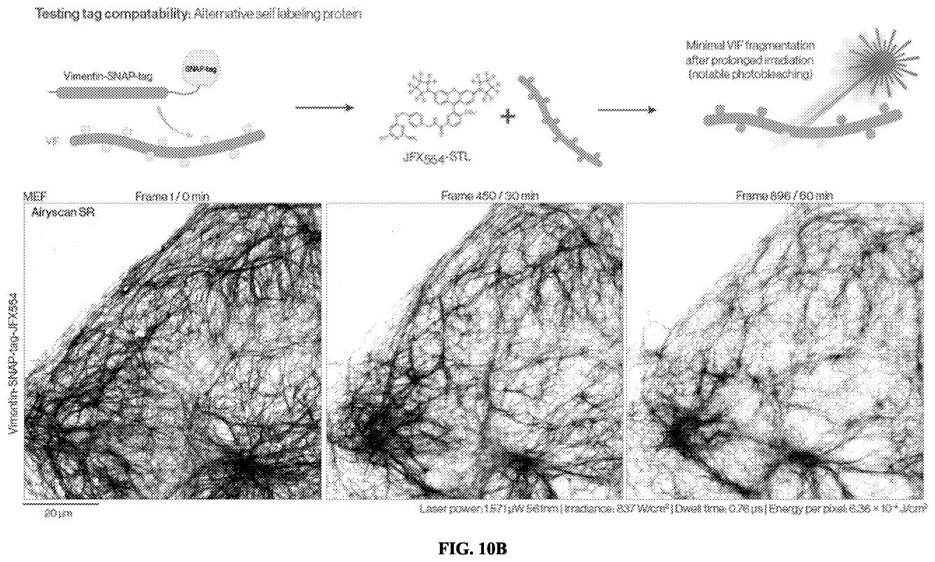

To test whether the photochemical mechanism requires the specific use of HaloTag® and chloroalkane ligands, two alternative strategies for localizing ROS generation to the filament surface were evaluated. First, a vimentin-SuperNova2 fusion was generated to produce VIF-localized ROS via a genetically encoded fluorescent photosensitizer. Upon prolonged 561 nm illumination, complete photobleaching of SuperNova2 was observed, but no observable VIF disassembly was detected when assessed using a co-expressed vimentin-mEmerald marker (FIG. 10A). Failure of the vimentin-SuperNova2 approach likely reflects two inherent limitations of fluorescent proteins compared to small-molecule dyes: substantially lower singlet oxygen and superoxide quantum yields and chromophores that are shielded by the β-barrel structure, potentially limiting ROS diffusion to nearby filament targets. Second, vimentin-SNAP-tag® fusions were labeled with the JFX554 SNAP-tag® ligand and cells were subjected to high-power 561 nm illumination. In this case, minimal and delayed fragmentation was observed, occurring only after extended exposure and substantial photobleaching (FIG. 10B). The reduced efficiency relative to HaloTag® may reflect slower ligand binding kinetics, electrostatic differences between tags (pI 9.6 for SNAP vs. 4.9 for HaloTag), or less favorable spatial positioning of the dye relative to the filament surface.

Taken together, these results define the spatial, structural, and mechanistic boundaries of the FilaBuster system. Efficient disassembly requires not only ROS production, but also precise positioning of the photosensitizer at native IFs. The failure of alternative tag systems and the resistance of unrelated cytoskeletal structures to equivalent light doses underscore that FilaBuster is not a generic photosensitizer platform, but a designed system for targeted, efficient, and selective fragmentation of inherently ROS-sensitive IFs.

### Example 23: Light-Induced VIF Fragmentation is Associated with Increased Vimentin Methionine Oxidation

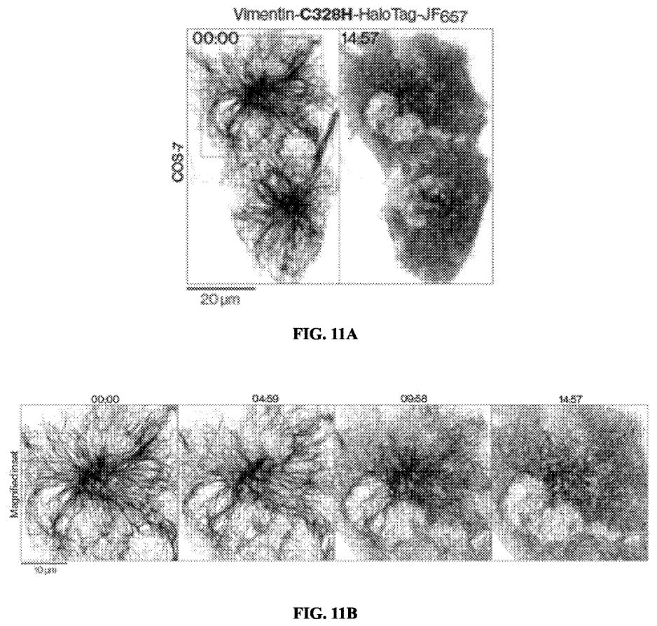

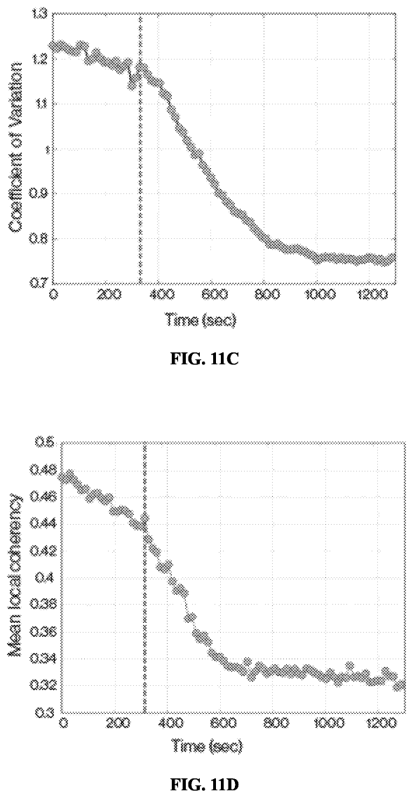

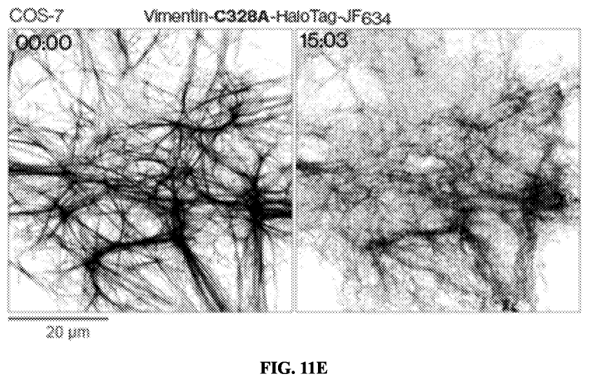

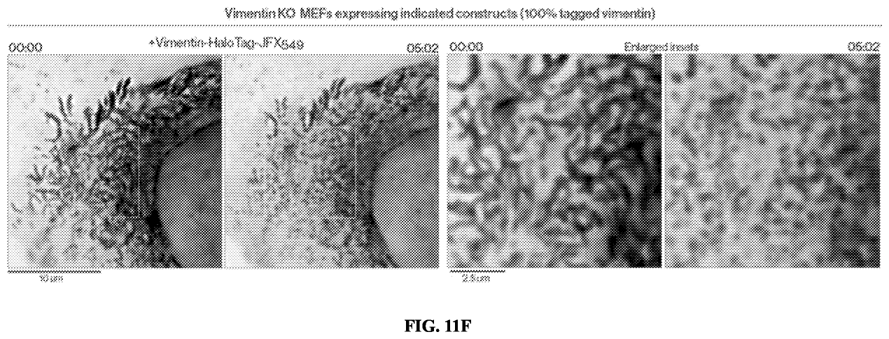

Given the well-established sensitivity of vimentin to oxidative stress, and extensive evidence implicating its single cysteine residue (C328) in redox-mediated filament disassembly, it was initially hypothesized that FilaBuster operates by targeting this residue. However, C328 proved dispensable. Vimentin-HaloTag® protein constructs harboring either a C328A or C328H mutation disassembled upon photosensitizer activation with kinetics indistinguishable from wild-type. This held true both in cells with an untagged endogenous vimentin background and in vimentin-null MEFs reconstituted exclusively with mutant constructs, confirming that filament fragmentation does not require modification at C328 (FIG. 11A-11I).

Having ruled out cysteine as the critical target, it was considered whether direct photochemical crosslinking between dye and filament might explain the effect, since a light-induced reaction between BSA and rhodamine dyes containing cyclic amine substituents has been observed in vitro. However, robust VIF fragmentation was observed with rhodamine dyes containing acyclic amine substituents, such as SiTMR, as well as with the carbofluorescein (CFl) HaloTag® ligand, which lacks alkylamines entirely, arguing against covalent adduct formation as a general mechanism.

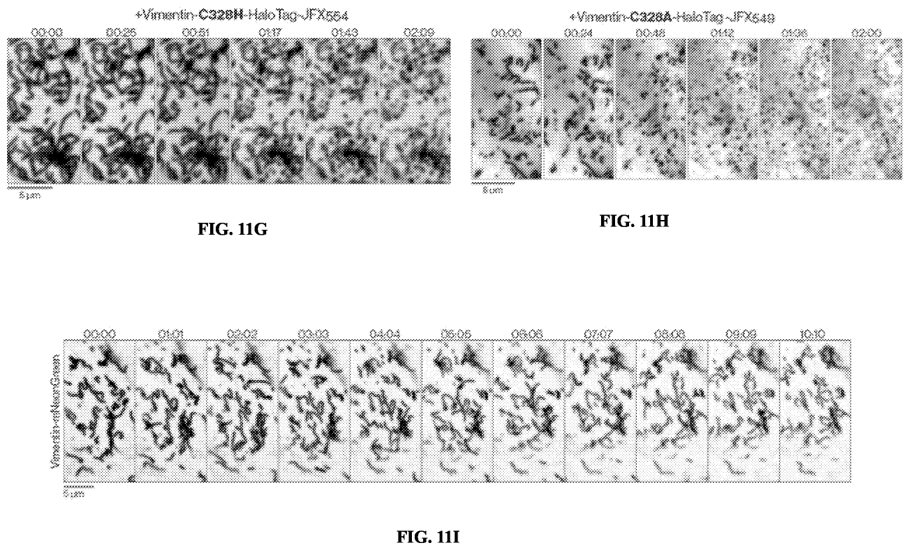

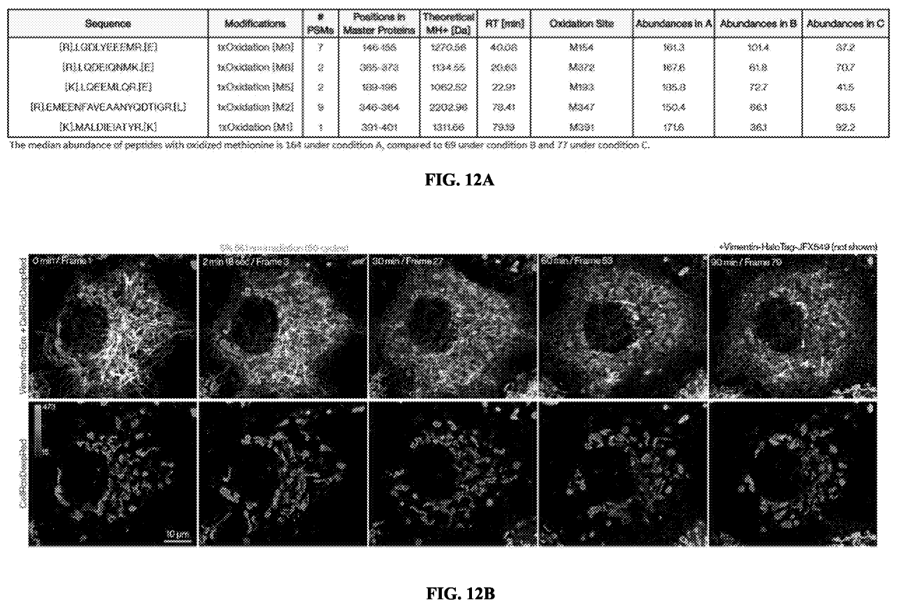

To directly assess oxidative modifications, mass spectrometry was performed on vimentin-HaloTag® protein U2-OS cells processed under three conditions: dye labeling with irradiation, dye labeling without irradiation, and unlabeled controls. Quantitative analysis revealed a striking increase in oxidized methionine residues in the irradiated group across multiple sites (FIG. 12A). This pattern is consistent with targeted ROS generation at the filament surface and suggests that localized subunit oxidation likely underlies disassembly. Of note, fluorescent detection of cellular ROS using CellROX Deep Red showed no measurable increase in signal following FilaBuster activation (FIG. 12B), suggesting minimal oxidative spillover and reinforcing the idea that disassembly is driven by highly localized photochemical events.

Together, these results indicate that FilaBuster operates by generating ROS at the filament surface, which in turn oxidizes vimentin subunits—primarily at methionine residues—triggering filament destabilization. This mechanism is not dependent on C328, does not involve bulk oxidative stress outside of the target filament, and can be tuned by varying irradiation parameters and HaloTag® ligand identity.

### Example 24: FilaBuster Enables Spatially Controlled VIF Fragmentation

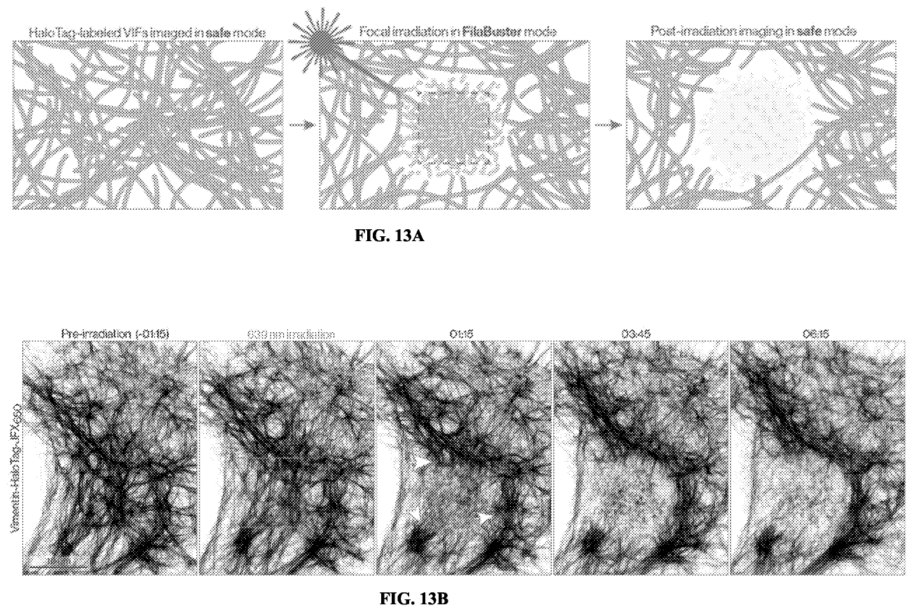

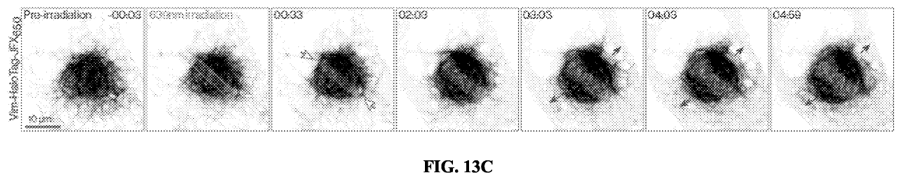

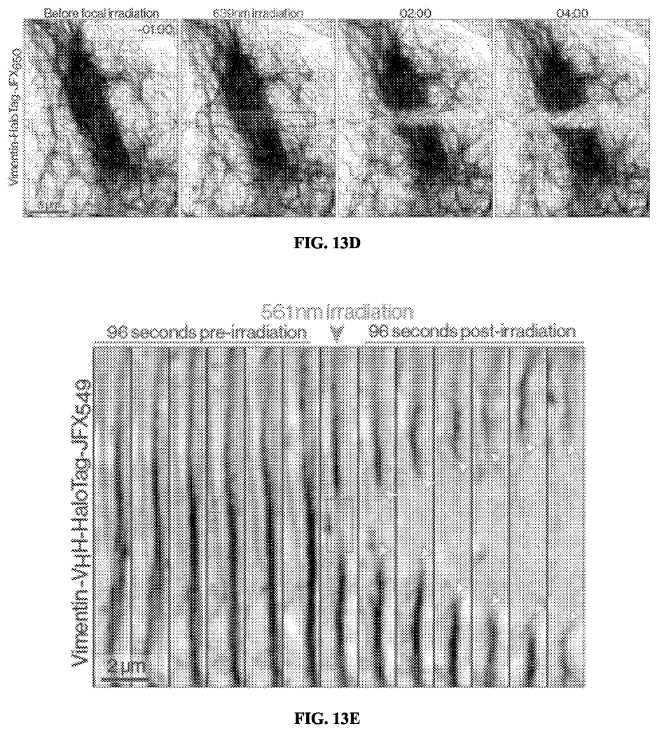

It was reasoned that by confining high-energy “FilaBuster” illumination to defined subcellular regions, localized fragmentation of the VIF network could be induced while preserving surrounding filaments (FIG. 13A), enabling spatiotemporally resolved perturbation of IF architecture—an approach that, until now, has not been possible.

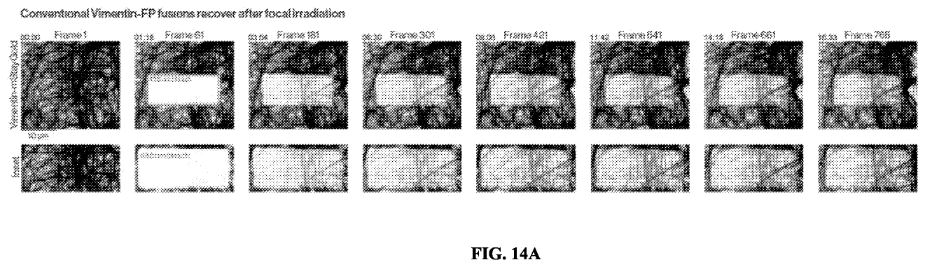

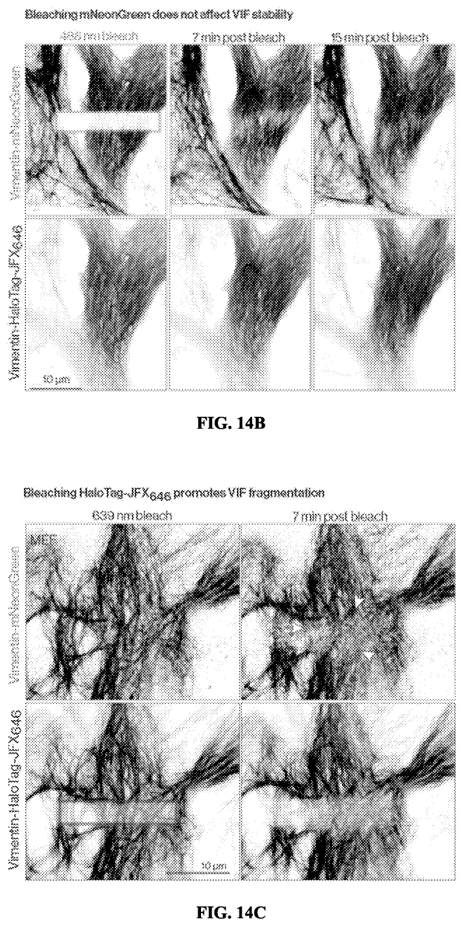

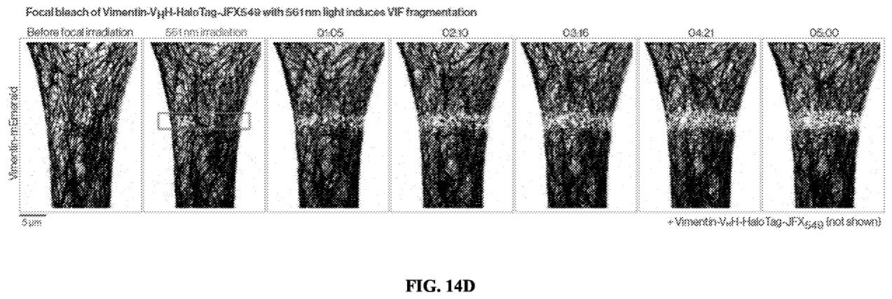

To test this, time-lapse imaging was performed on COS-7 cells stably expressing vimentin-HaloTag® protein and labeled with JFX650-HTL, a rhodamine-based dye with low photochemical reactivity under standard imaging conditions. Cells were initially imaged under low-power standard conditions to establish a stable baseline. At a defined time point, a ~10×10 μm region of interest (ROI) was exposed to focused 639 nm illumination at higher irradiance to trigger FilaBuster mode activation. Imaging then resumed under standard, low-power imaging conditions to monitor structural changes post-irradiation. Within one minute, vimentin IFs within the irradiated ROI underwent rapid and complete disassembly, while filament structures outside the targeted area remained intact (FIG. 13B). Notably, VIF transport into the irradiated region was not observed, as is typically seen when photobleaching conventional vimentin fusion proteins (FIG. 14A), suggesting that the irradiated VIF breakdown products either immediately destabilize newly trafficked VIFs or interfere with local VIF trafficking.

Furthermore, focal illumination at wavelengths outside the FilaBuster ligand's excitation spectrum did not induce VIF disintegration. In cells co-expressing vimentin-mNeonGreen and vimentin-HaloTag-JFX646, 488 nm irradiation had no effect on VIF stability, while 639 nm irradiation drove robust fragmentation (FIG. 14B-14C).

Building on this spatial precision, it was next explored whether structurally diverse vimentin IF assemblies could be disrupted. By positioning focal irradiation windows over thick, bundled IF structures, brief and spatially confined 639 nm irradiation was shown to efficiently bisect perinuclear vimentin caps as well as thick peripheral VIF bundles (FIG. 13C-13D).

Focal VIF fragmentation was also achieved using the nanobody targeting strategy. With vimentin-VHH-HaloTag-JFX549, thin filament bundles were precisely severed with focal 561 nm illumination, leading to dramatic retraction of the free ends (FIG. 13E). To ensure that disassembly could be robustly visualized with minimal interference from imaging conditions, the experiment was repeated using a co-expressed vimentin-mEmerald reporter (FIG. 14D). This approach allowed monitoring of the same irradiation-induced disassembly process while exclusively using the stable mEmerald signal for before-and-after imaging. Since mEmerald is excited with the 488 nm laser line, which does not activate the photosensitizer, this method ensured that IF disassembly was induced solely by targeted irradiation and not by imaging-associated illumination.

All validated HaloTag® ligands from the global disassembly assays (FIG. 13E) supported localized filament disruption under focal irradiation. However, potent photosensitizer dyes such as JF₅₇₀-HTL posed challenges for spatial precision due to their high reactivity; even minimal light exposure after focal irradiation caused widespread filament loss.

### Example 25: Rapid, Localized Filament Scission Defines Early-Stage VIF Disassembly Kinetics

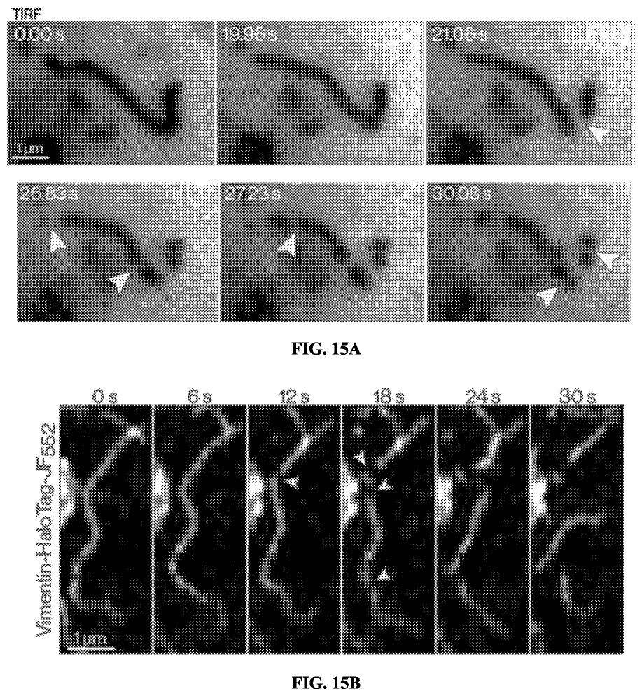

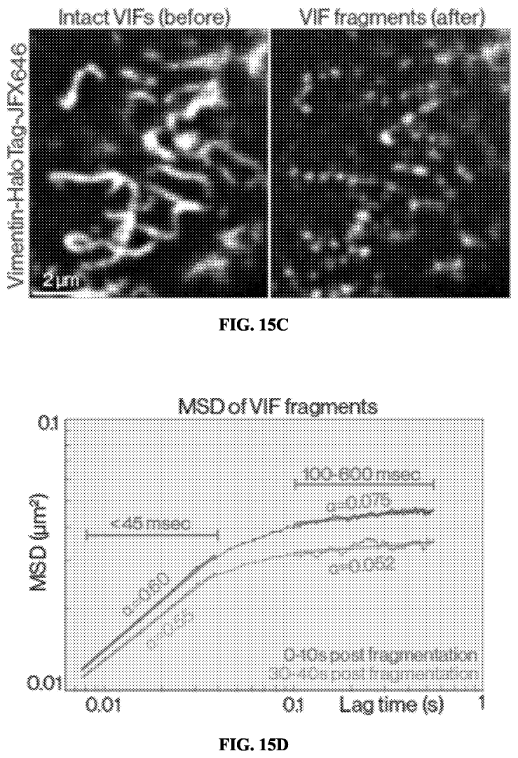

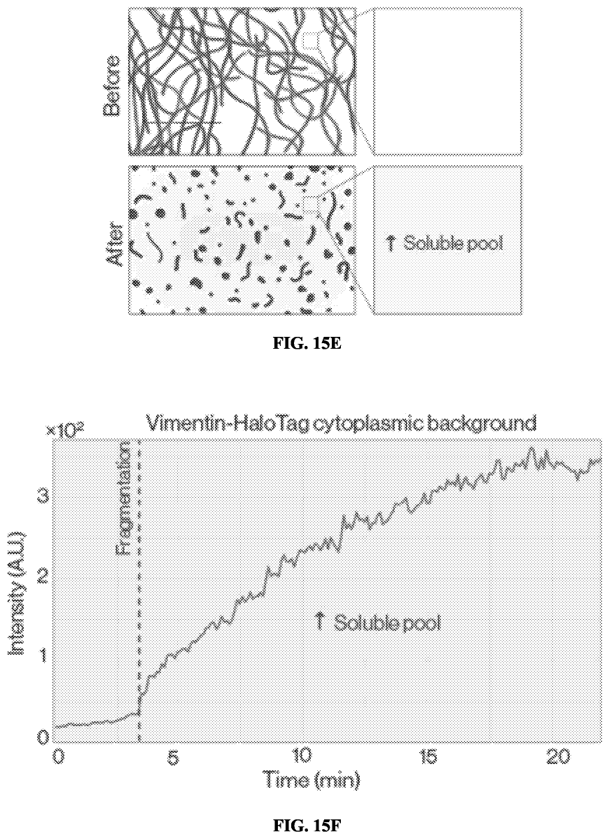

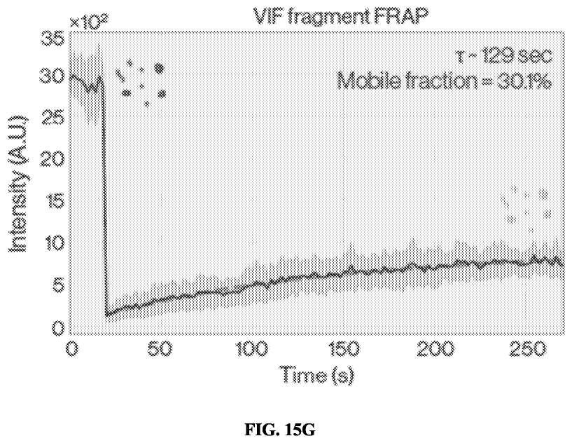

To define the kinetics and mechanical characteristics of FilaBuster-mediated disassembly, high-speed TIRF and confocal imaging was performed on labeled vimentin filaments before, during, and after illumination-induced fragmentation. Under continuous illumination, filaments initially appeared stable and phenotypically unremarkable. However, upon exceeding the threshold light dose, the sudden onset of filament disassembly was observed, typically initiated by 2-3 severing events, producing ~1 μm truncated filaments. Severing frequently occurred at points of high curvature, as seen in both TIRF and Airyscan recordings (FIG. 15A-15B). Truncated filaments persisted briefly before collapsing into minimal, insoluble fragments (FIG. 15C).

To characterize the behavior of newly formed VIF fragments, single-particle tracking was performed during the first few seconds post-fragmentation (FIG. 15D). Mean-squared displacement (MSD) analysis revealed a two-phase motility profile. At short lag times (<45 ms), fragments exhibited subdiffusive motion (α≈0.60, Deff≈0.22 μm²/s), consistent with constrained diffusion through the cytoplasm. At longer lag times (100-600 ms), the MSD plateaued (α≈0.075). This motility state transition is interpreted as a consequence of polydisperse VIF fragments attaching to and detaching from the cytoplasmic meshwork of actin filaments and microtubules. Concurrent with VIF fragmentation, a global rise in diffuse, cytoplasmic vimentin-HaloTag® protein signal was observed, even in regions previously devoid of assembled VIFs; an effect attributed to a dramatic increase in the typically negligible soluble vimentin tetramer pool (FIG. 15E-15F). Together, these data suggest that FilaBuster-induced disassembly generates at least two distinct end products: spatially confined, truncated VIF fragments and a rapidly diffusing soluble vimentin pool—each with potentially distinct cellular effects.

### Example 26: VIF Fragments Persist in the Cytoplasm and Block New Filament Assembly

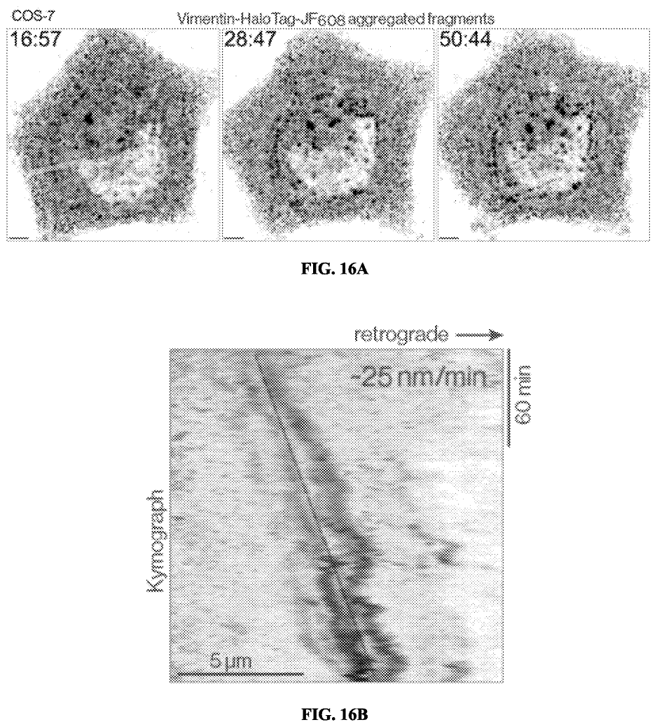

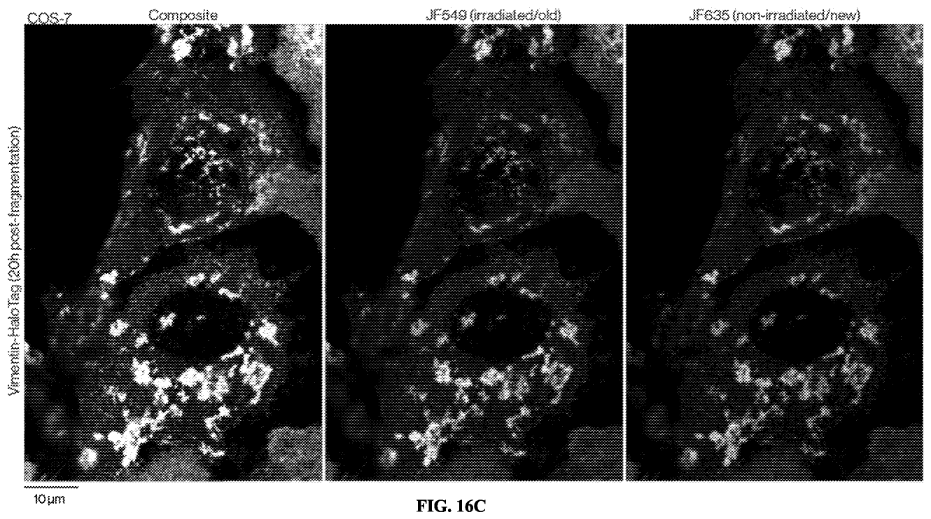

While the early phase of disassembly results in widespread filament fragmentation and redistribution of soluble subunits, it was next assessed whether the network could reassemble after this acute disruption. To determine whether VIF fragments retained dynamic subunit exchange with the soluble pool, fluorescence recovery after photobleaching (FRAP) was performed on stationary fragments ~10 minutes after disassembly (FIG. 15G). Over 250 seconds, ~30% recovery was observed with a characteristic time (t) of ~129 seconds, indicating that fragments retain the capacity for subunit exchange, but not for productive reassembly. Instead, over the subsequent hours, these fragments gradually coalesced, migrating toward the perinuclear region at ~25 nm/min and condensing into dense, immobile aggregates ranging from 500 nm to 5 μm in diameter (FIG. 16A-16B).

To test whether this assembly block extended to newly synthesized vimentin, a two-color pulse-chase assay was performed. Cells were first labeled with JF549 to mark the pre-existing vimentin-HaloTag® protein pool, then immediately subjected to 561 nm light-induced disassembly. After a 20-hour recovery period, the cells were labeled with JF635-HTL to visualize any vimentin-HaloTag® proteins synthesized after irradiation. If filament assembly were possible, these newly translated, non-irradiated subunits would be expected to form a distinct filamentous network. Instead, JF₆₃₅-labeled vimentin failed to assemble and co-localized with the original JF₅₄₉-labeled aggregates, despite never being exposed to light (FIG. 16C). Together, these results demonstrate that FilaBuster not only irreversibly fragments irradiated VIFs, but also imposes a dominant, long-lasting block on reestablishing a filamentous network, even in the presence of abundant, newly synthesized vimentin.

### Example 27: Acute VIF Fragmentation Reveals Immediate Effects on Cell Morphology and Cytoskeletal Organization

Much of the current understanding of IF function has relied on comparisons between wild-type and IF knockout cells. Although valuable, knockout approaches have important limitations. First, acute effects of IF loss cannot be captured, preventing direct observation of its immediate mechanical and structural consequences. Second, knockout cells may develop compensatory adaptations that obscure the primary roles of IFs and complicate functional interpretation. Here, FilaBuster is leveraged to acutely disassemble vimentin IFs in living cells, enabling direct tracking of cellular phenotypes before, during, and immediately after filament disruption. Using direct IF disassembly with FilaBuster, several immediate changes in cellular phenotype are uncovered.

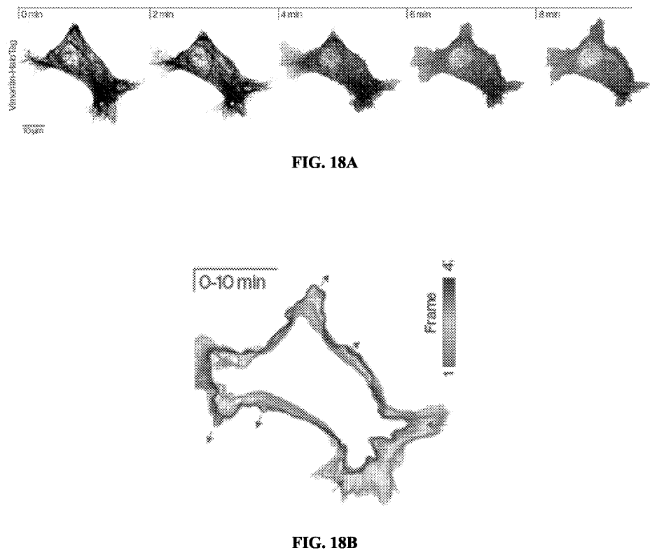

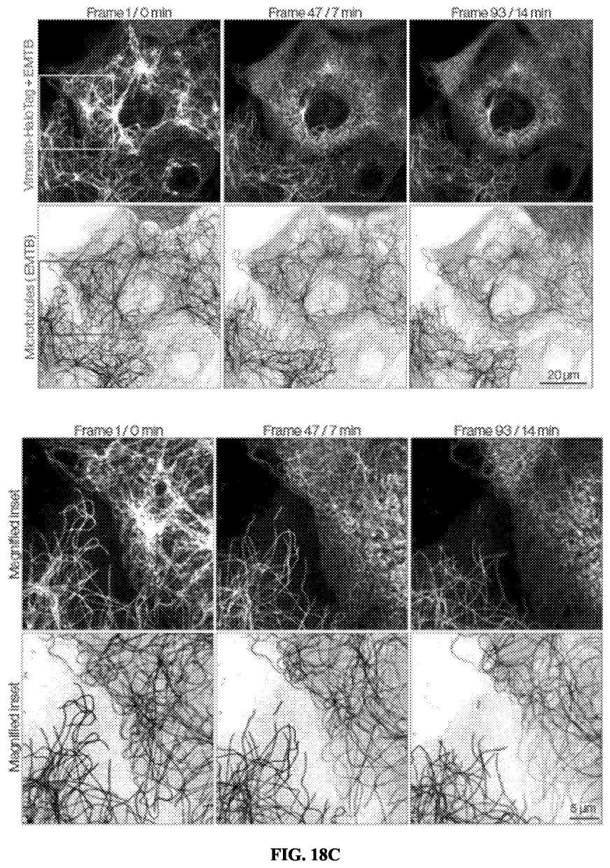

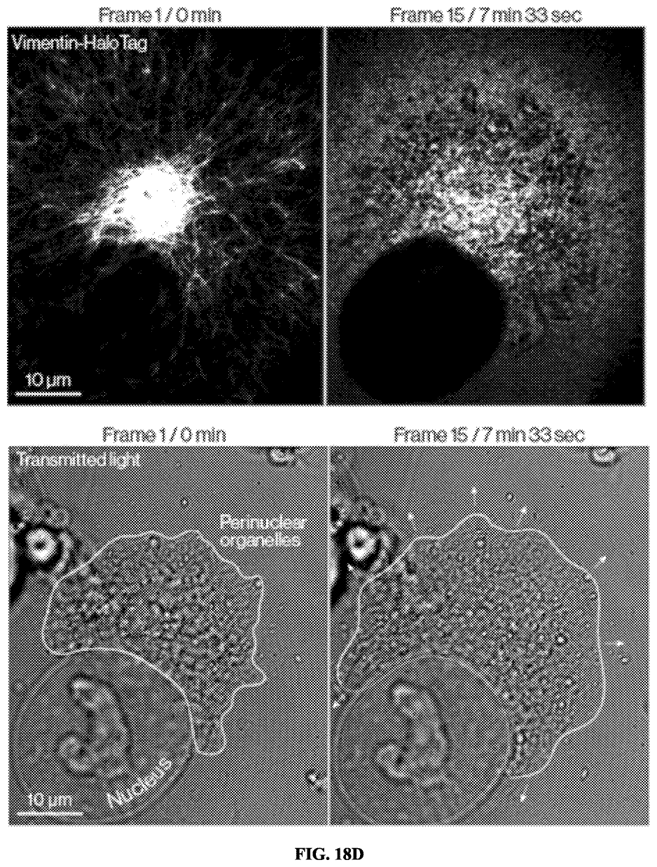

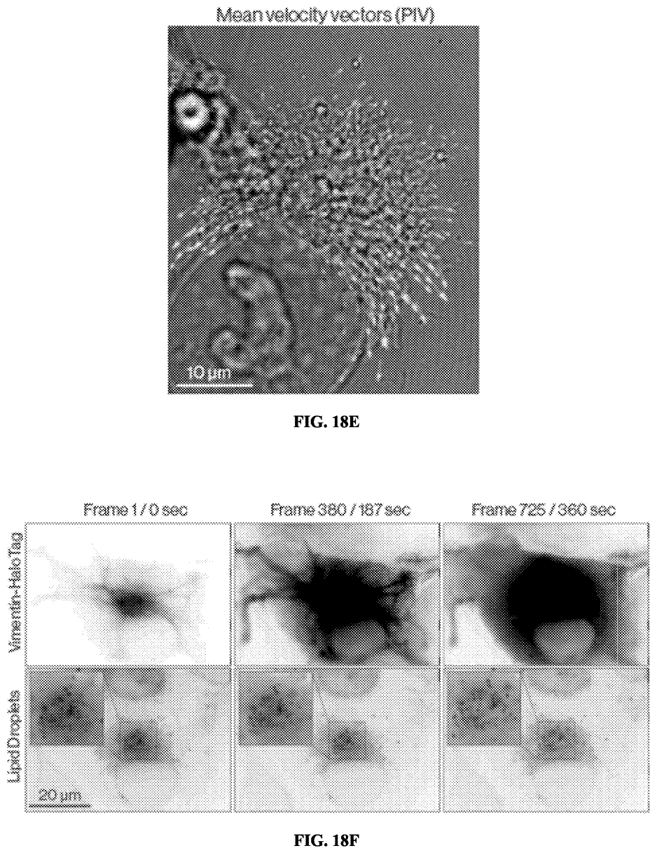

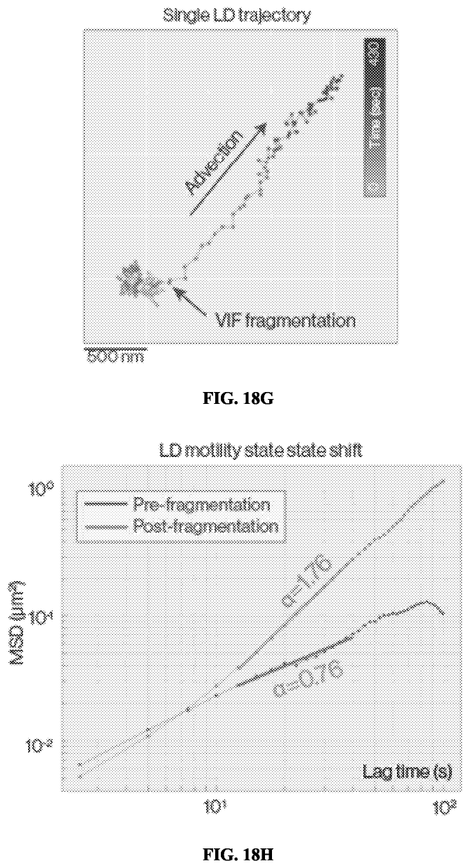

Acute vimentin IF disassembly triggered a retraction of asymmetrical cellular extensions and a gradual cell rounding (FIG. 18A). Cells transitioned from an elongated, irregular morphology to a more isotropic, circular shape (FIG. 18B). Notably, similar cell morphology transitions have been reported in cells microinjected with high concentrations of dominant negative vimentin mimetic peptides. This shift in cell architecture likely reflects changes to the organization of actin and microtubule cytoskeletons, with which VIFs make frequent interactions.

Indeed, VIF fragmentation elicited immediate responses in the actin cytoskeleton. In many cells, a rapid increase in actin retrograde flow was observed, driving centripetal movement of fragmented IFs toward the perinuclear region. In other cells, actin dynamics remained largely unchanged. Over longer timescales post-fragmentation, VIF fragments were observed accumulating along stress fibers and focal adhesions. Notably, vimentin droplets that form upon recovery from hypotonic stress or expression of the vimentin Y117L ULF mutant display similar stress fiber localization. Stress fibers that became heavily decorated with VIF fragments often destabilized at later time points, resulting in rapid cell shape changes, an effect attributed to either loss of structural scaffolding provided by intact IF networks or direct interactions between VIF fragments and actin filaments, which could displace or inhibit actin-binding and regulatory proteins.

The immediate effects of acute vimentin IF disassembly on microtubules were next examined, motivated by extensive literature describing their close physical and functional interactions. VIFs globally align with microtubules and rely on microtubule motor-based transport for proper positioning within the cytoplasm. The extent to which microtubule networks rely on VIFs for structural support is less clear. Indeed, microtubule networks appear structurally normal in vimentin knockout cells, leading to the prevailing view that microtubules do not require IFs for stability. However, vimentin 1A mimetic peptide microinjection has been observed to induce dramatic microtubule depolymerization. Here, taking advantage of the spatiotemporal control afforded by the FilaBuster approach, a nuanced relationship is uncovered that reconciles these seemingly contrasting observations. In cells with sparse vimentin networks, IF disruption had little to no effect on microtubule integrity (FIG. 18C). By contrast, in cells with dense, highly entangled vimentin IF networks, acute IF disassembly triggered rapid microtubule destabilization and fragmentation.

These findings indicate a conditional dependence of microtubules on vimentin IFs: although microtubule networks can form normally in the chronic absence of vimentin, their stability becomes critically reliant on IFs when the two filament systems are extensively co-aligned and interconnected. This conditional dependence highlights a previously unappreciated functional coupling between these cytoskeletal systems-one that is uniquely revealed by FilaBuster-mediated acute perturbation but easily overlooked using traditional knockout approaches.

### Example 28: Acute Vimentin IF Disassembly Triggers Rapid Redistribution of Perinuclear Organelles

Vimentin IFs form dense perinuclear networks that constrain organelles and regulate their positioning. While previous studies have shown that chronic depletion of vimentin leads to changes in average organelle distributions across populations, the direct effect of rapid and selective VIF disassembly on organelle position and dynamics has never been observed and analyzed in real time.

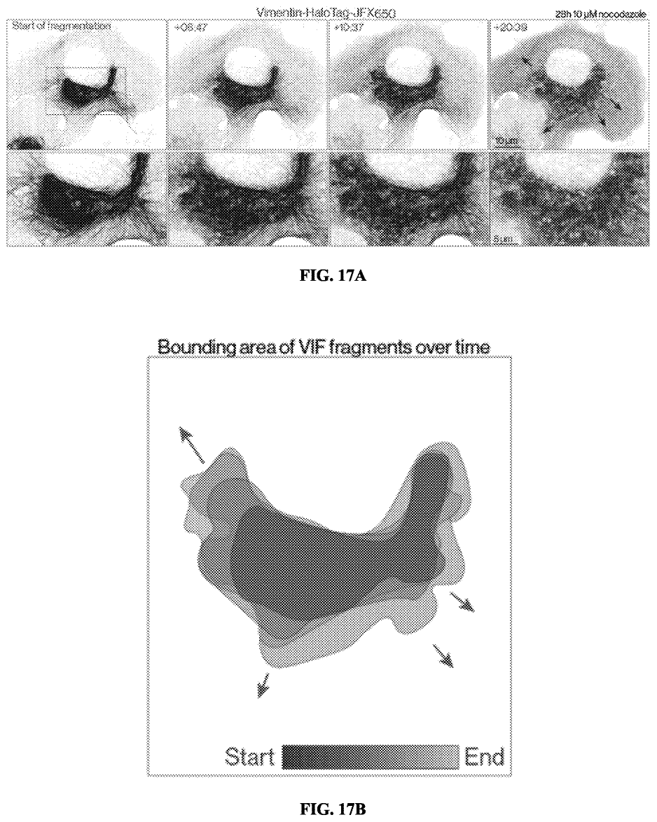

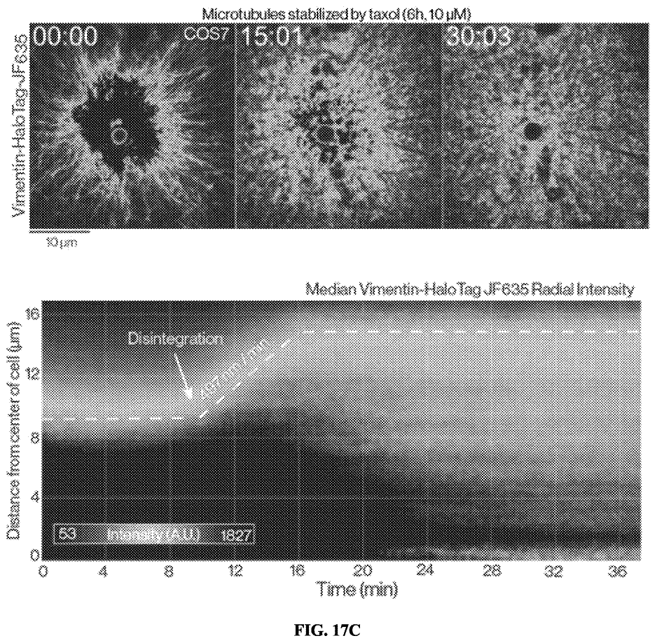

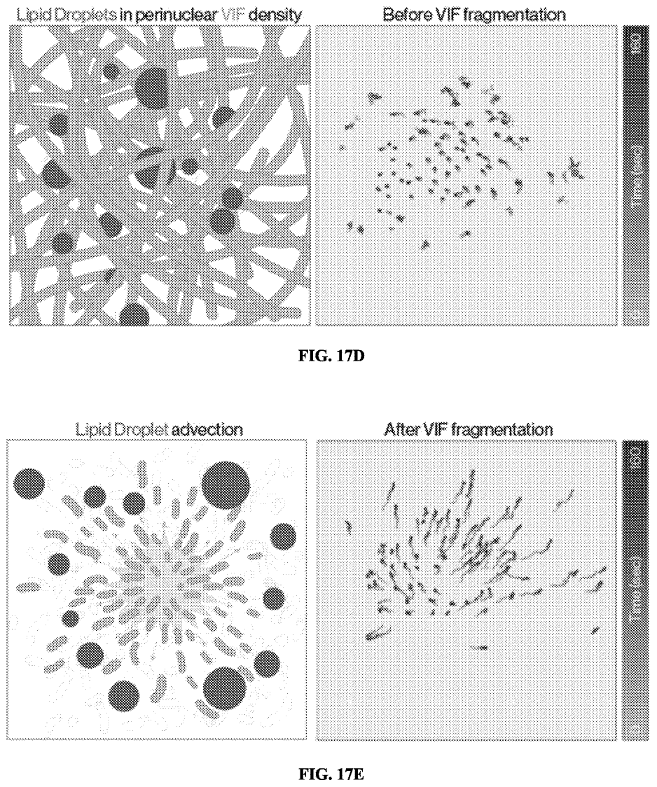

Here, using vimentin-HaloTag®Protein-JF₆₃₄, a perinuclear VIF density was irradiated with 639 nm illumination, and the effect on organelles was examined by gentle transmitted light imaging. Upon reaching threshold illumination levels, VIF fragments rapidly surged outwards in a radially propagating wave, traveling at ~500 nm/sec (FIG. 17A-17C). Organelles that were previously entangled within the dense VIF network simultaneously lurched outwards, undergoing radial advection, resulting in a more uniform membrane mass distribution within the cytoplasm and confirming a direct mechanical role for VIFs in subcellular organelle positioning (FIG. 18D-18E). Single-particle tracking of perinuclear, VIF-embedded lipid droplets further elucidated this effect, wherein a dramatic motility state transition from subdiffusive (α=0.76) to directed outward advection (α=1.76) was observed immediately following VIF disruption (FIG. 17D-17E, FIG. 18F-18H). These observations demonstrate that vimentin IFs function as a mechanical barrier, confining organelles within the perinuclear region. Acute loss of this barrier induces a rapid, advective redistribution of intracellular components, an effect masked in traditional knockout models where IF loss occurs gradually over time.

### Example 29: Controlled Disassembly of GFAP, Desmin, Peripherin, and Cytokeratin Filaments by Activation of a HaloTag-Targeted Rhodamine Dye

Given the shared susceptibility to oxidant-mediated disassembly reported across IF subtypes, it was initially hypothesized that FilaBuster could be a universal approach for IF fragmentation, wherein filament specificity would simply be defined by the identity of the HaloTag® targeting protein (e.g., IF protein or nanobody). Having thus far established this approach using VIFs as a simple proof-of-concept model system, it was next evaluated whether FilaBuster could in fact be generalized to other IF subtypes.

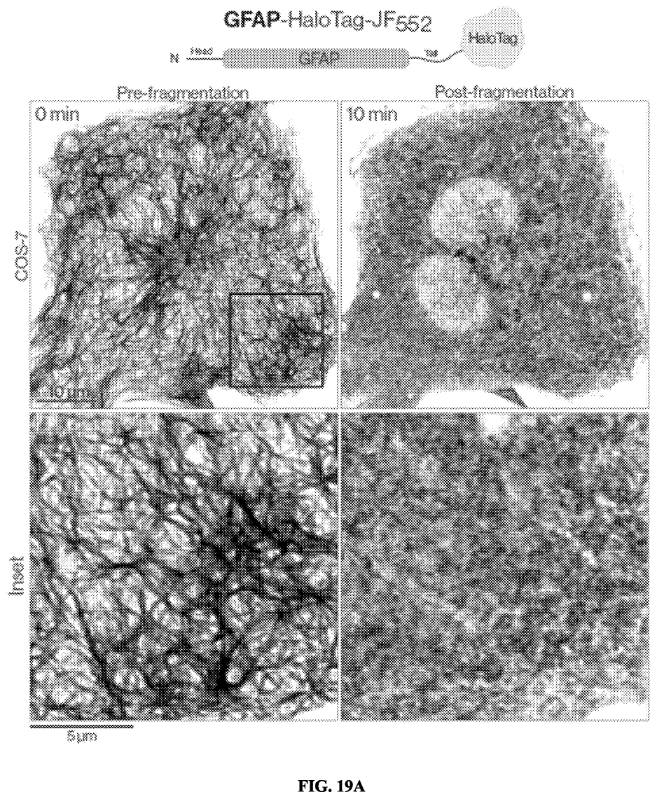

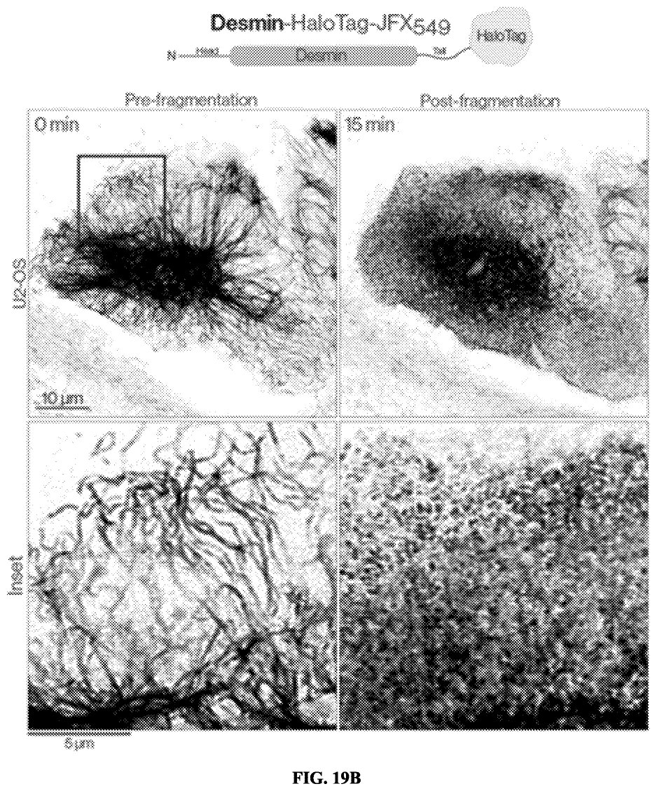

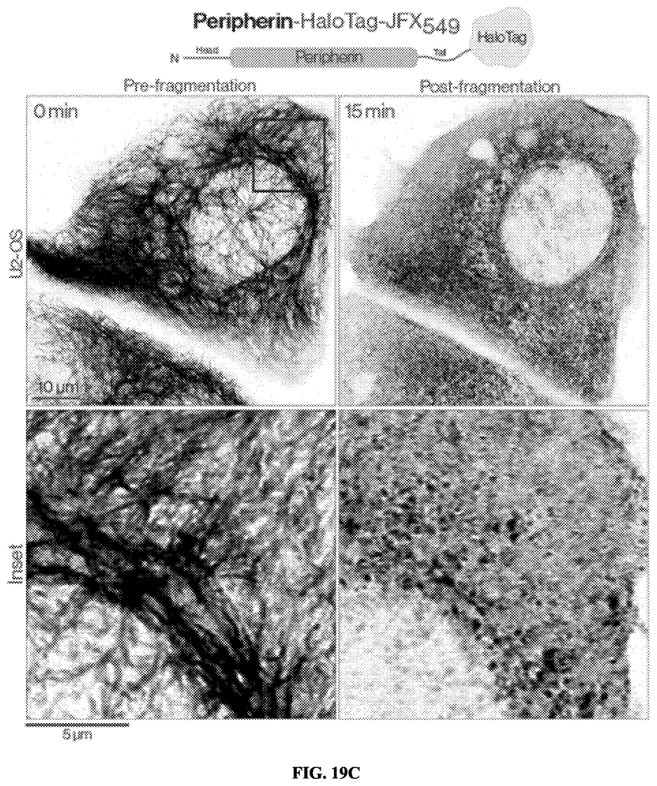

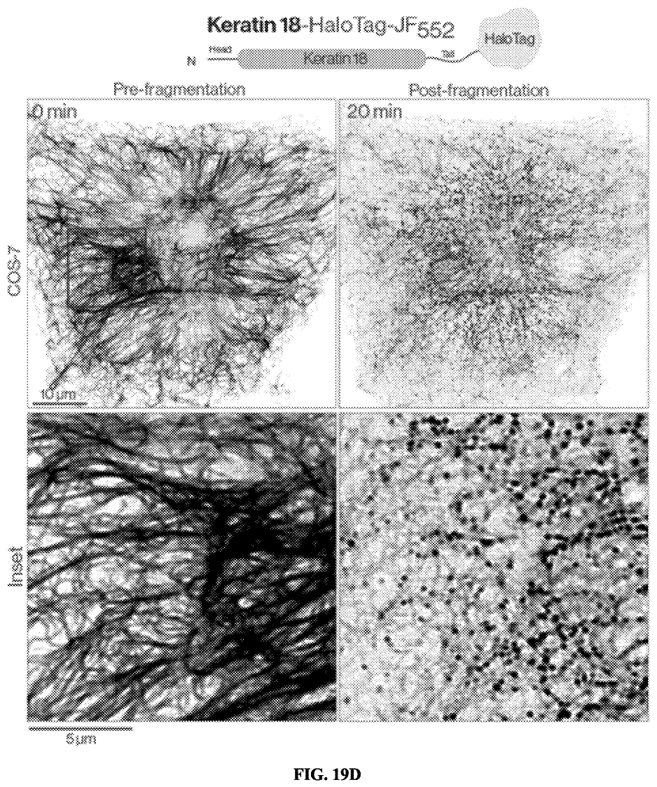

To this end, C-terminal HaloTag® protein fusions of additional Type III IF proteins—GFAP, desmin, and peripherin—were expressed. These constructs were then electroporated into either COS-7 or U2-OS cells, labeled with optimal rhodamine HTLs based on earlier dye benchmarking studies (FIG. 6E), and Airyscan images were acquired confirming that all fusions assembled phenotypically normal networks. Next, upon increased 561 nm illumination, robust IF fragmentation was observed in all cases, with similar kinetics and fragmentation patterns to those observed in proof-of-concept experiments with VIFs (FIG. 19A-19C).

As Type III IF proteins frequently form mixed filament networks, it was next tested whether targeting one Type III IF protein with FilaBuster would also induce fragmentation of a separately labeled, co-expressed Type III. In cells co-expressing vimentin-HaloTag-JF552 and Desmin-EGFP, irradiation of the HaloTag® ligand triggered disassembly of both networks. Similarly, focal 639 nm irradiation of GFAP-HaloTag-JFX650 induced simultaneous fragmentation of vimentin-mApple-labeled VIFs. Notably, in both cases, direct irradiation of the conventionally tagged IF alone had no effect on filament stability, confirming that disassembly only occurs when a photosensitizer ligand is activated.

Finally, it was examined whether FilaBuster could be extended to other cytoplasmic IF classes. As proof of principle, focus was placed on the abundant and well characterized type I cytokeratin, keratin 18. COS-7 cells were electroporated with a keratin 18-HaloTag® protein construct, labeled with JF552-HTL, and a dense cytoplasmic network was observed, consistent with efficient heterodimerization with endogenous type II keratins. Upon sustained 561 nm irradiation, clear loss of keratin IFs was observed; however, the disassembly process appeared qualitatively distinct from that of Type III IFs. Rather than abruptly disintegrating into minimal fragments scattered throughout the cytoplasm, as seen with vimentin (FIG. 15A-15C), keratin filaments gradually developed discrete, granular structures along their length, reminiscent of the structural reorganization reported upon orthovanadate treatment. Over several minutes, these puncta increased in size and intensity, ultimately depleting keratin from the filament network until only these structures remained (FIG. 19D).

### Example 30: Dose-Dependent Induction of Vimentin Intermediate Filament Fragmentation without Global Phototoxicity

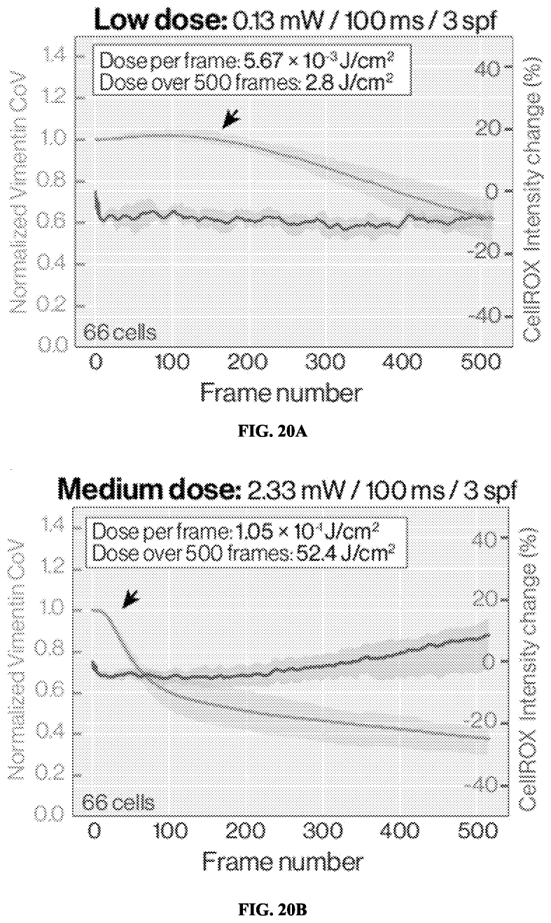

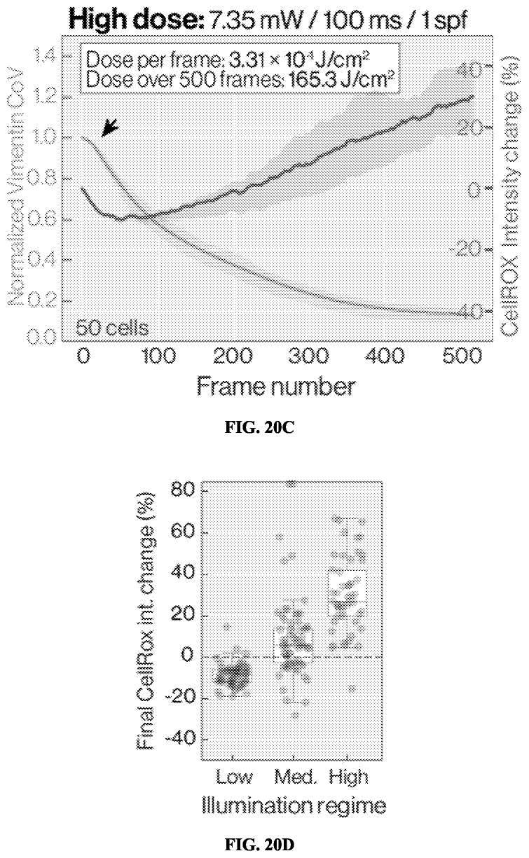

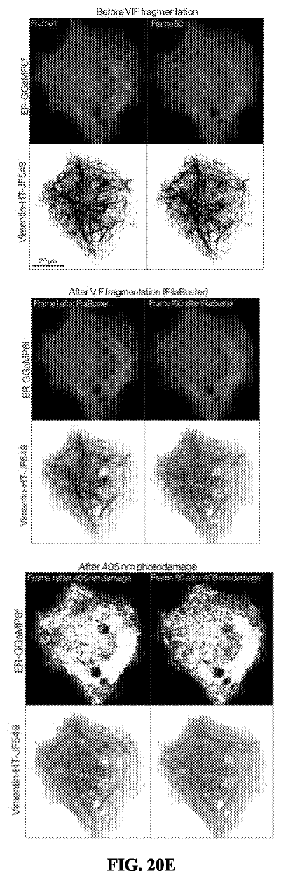

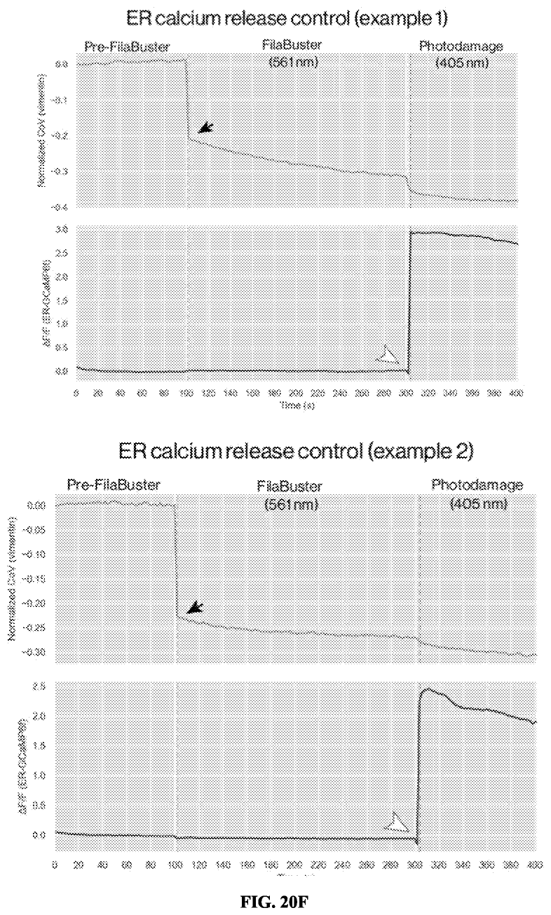

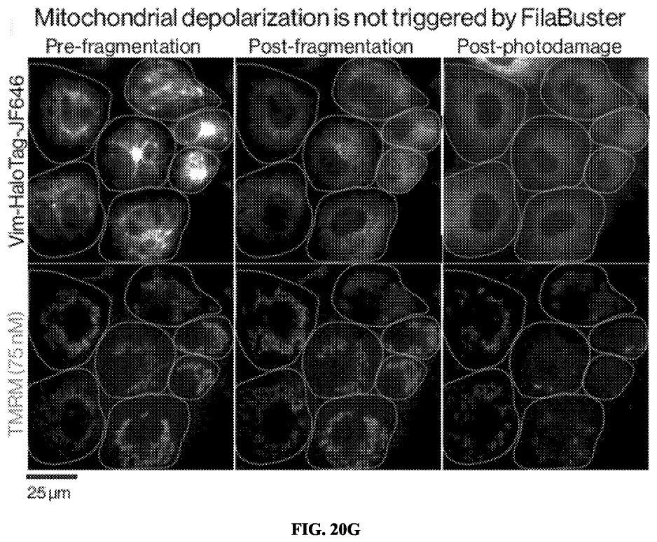

To define illumination conditions that induce vimentin intermediate filament (VIF) fragmentation while minimizing cellular stress, a dose-response analysis was performed using widefield 561-nm illumination in vimentin-HaloTag® protein knock-in COS-7 cells labeled with a rhodamine-derived HaloTag® ligand and the reactive oxygen species (ROS) indicator CellROX Green (FIG. 20A-20D). VIF fragmentation was quantified over time using a normalized coefficient of variation (CoV) metric, while oxidative stress was assessed by changes in CellROX fluorescence intensity. Under low-dose illumination, VIF fragmentation was initiated after approximately 150 illumination cycles without a detectable increase in ROS signal (FIG. 20A), indicating that filament disruption can occur in the absence of measurable global oxidative stress. Increasing illumination dose accelerated the onset of VIF fragmentation (FIG. 20B-20C) but was accompanied by a progressive increase in CellROX signal, indicating elevated oxidative stress at higher energy exposures (FIG. 20D). Endpoint analysis across illumination regimes confirmed minimal ROS generation at low doses and substantially higher oxidative stress at medium and high doses (FIG. 20D).

To assess whether VIF fragmentation under threshold illumination conditions triggers other forms of cellular damage, additional functional readouts were examined. Endoplasmic reticulum (ER) calcium homeostasis was monitored using an ER-anchored, cytosol-facing calcium indicator (ER-GCaMP6f). Global VIF fragmentation induced by 561-nm illumination did not elicit ER calcium release (FIG. 20E-20F), whereas a positive-control photodamage stimulus using 405-nm irradiation produced a robust calcium signal consistent with ER calcium leakage. Similarly, mitochondrial membrane potential was assessed using the dye TMRM. Following VIF fragmentation, mitochondrial TMRM fluorescence was preserved, indicating maintained membrane potential, while 405-nm photodamage resulted in rapid mitochondrial depolarization (FIG. 20G). Together, these data demonstrate that VIF fragmentation induced under appropriate illumination conditions is not accompanied by global oxidative stress, ER dysfunction, or mitochondrial depolarization.

### Example 31: Intermediate Filament Fragmentation does not Grossly Perturb Other Cytoskeletal Systems or Organelle Dynamics

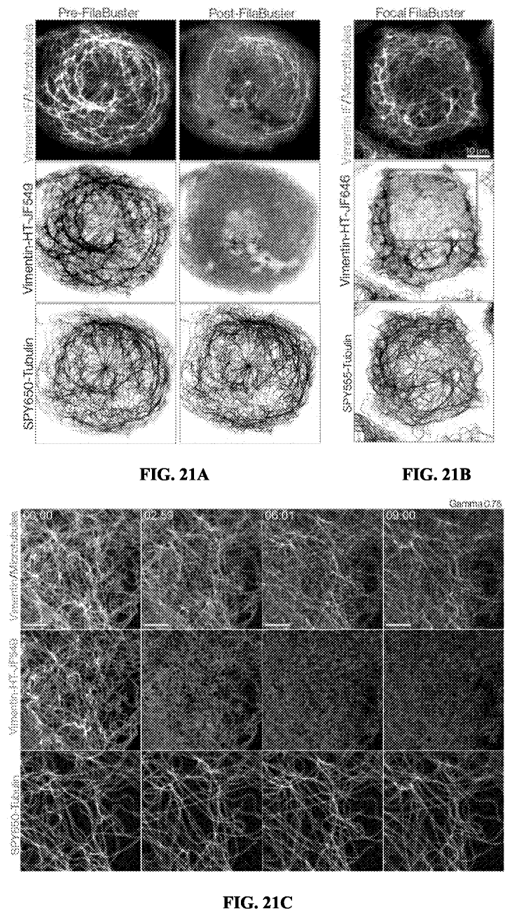

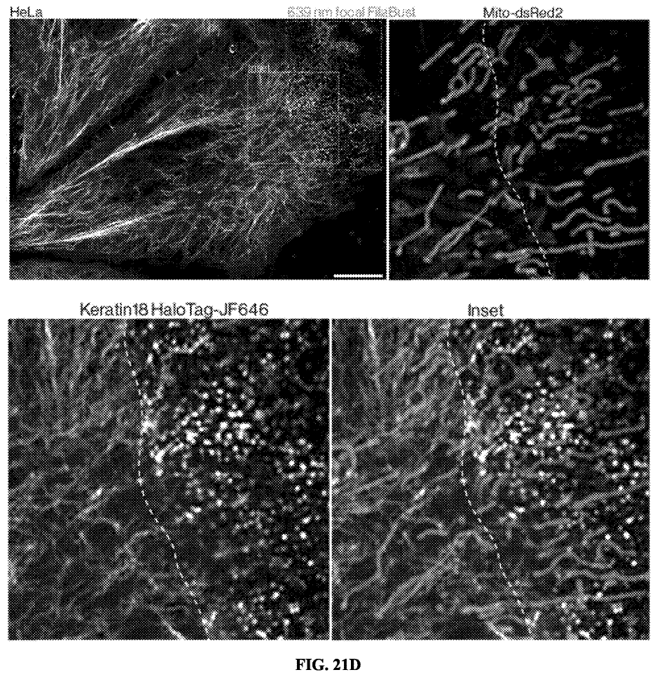

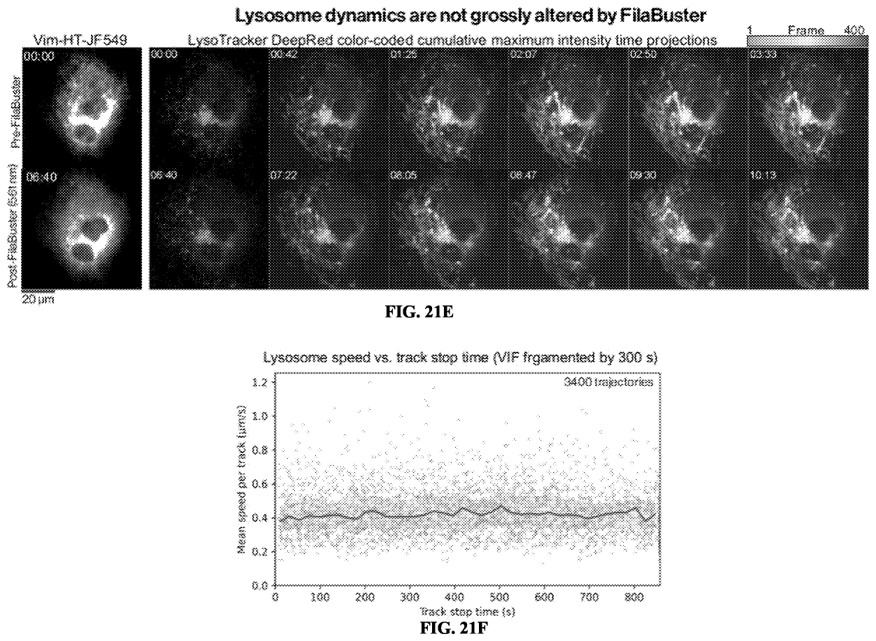

The effects of VIF fragmentation on other subcellular structures were examined to assess specificity. In vimentin-HaloTag® protein knock-in COS-7 cells labeled for both vimentin and endogenous microtubules, global 561-nm illumination induced widespread VIF fragmentation without disrupting the overall organization of the microtubule network (FIG. 21A). Focal illumination experiments further demonstrated that localized VIF fragmentation could be achieved while preserving microtubule continuity both within and outside the irradiated region (FIG. 21B). Time-lapse imaging revealed progressive loss of vimentin filament continuity with microtubule morphology remaining intact over the same interval (FIG. 21C).

The impact of intermediate filament fragmentation on mitochondria and lysosomes was also evaluated. In HeLa cells expressing keratin18-HaloTag® protein and a mitochondrial matrix marker, focal 639-nm illumination induced local keratin filament disassembly without producing obvious mitochondrial morphological abnormalities in the irradiated region (FIG. 21D). Lysosome motility was assessed in vimentin-HaloTag® protein knock-in COS-7 cells labeled with LysoTracker Deep Red. Comparison of lysosome trajectories before and after global VIF fragmentation showed no gross alteration in lysosome movement patterns (FIG. 21E). Automated tracking of thousands of lysosome trajectories revealed no substantial reduction in mean lysosome speed following VIF fragmentation (FIG. 21F). These observations indicate that acute intermediate filament fragmentation does not broadly impair microtubule organization, mitochondrial morphology, or lysosome motility.

### Example 32: Cells Remain Viable and Reestablish the Vimentin Network Following Global Intermediate Filament Fragmentation

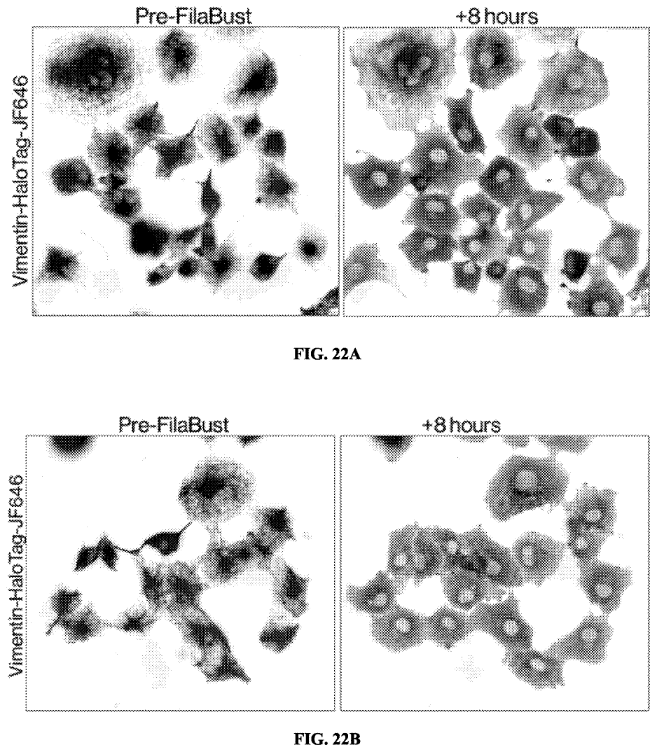

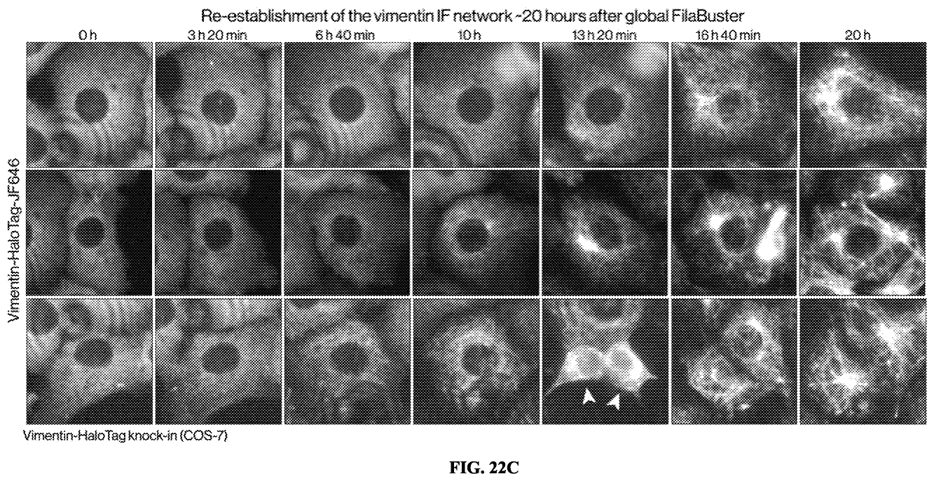

To examine long-term cellular responses to intermediate filament disruption, vimentin-HaloTag® protein knock-in COS-7 cells were subjected to global 640-nm illumination sufficient to fragment the endogenous VIF network and were then imaged over extended time periods (FIG. 22A-22C). Prior to illumination, vimentin was organized as an extended filamentous network with prominent perinuclear enrichment (FIG. 22A). Immediately following fragmentation, vimentin fluorescence redistributed into dispersed, punctate structures throughout the cytoplasm (FIG. 22B). Despite this dramatic reorganization, cells remained spread, adherent, and morphologically intact over subsequent hours.

Long-term time-lapse imaging revealed a gradual recovery of filamentous vimentin organization (FIG. 22C). Over the course of approximately 20 hours, dispersed vimentin fragments progressively reorganized into a perinuclear network and subsequently into an extended filamentous array resembling the pre-fragmentation state. During this recovery period, cells were observed to undergo mitosis, indicating that cells not only survive acute VIF fragmentation but are capable of progressing through cell division and reestablishing the endogenous intermediate filament network.

### Example 33: Photochemical Effects Depend on Subcellular Localization of the HaloTag-Anchored Photosensitizer

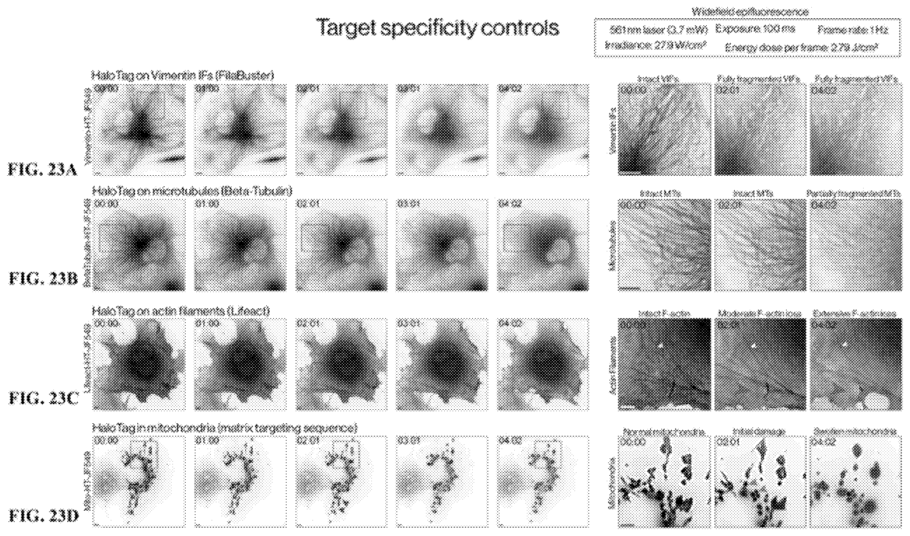

To determine whether identical illumination conditions produce distinct outcomes depending on the subcellular location of the photosensitizer, HaloTag® ligands were targeted to different cellular structures and imaged under the same widefield 561-nm illumination used for VIF fragmentation (FIG. 23A-23D). When HaloTag® protein was localized to vimentin intermediate filaments, rapid and progressive fragmentation of the filament network was observed, transitioning from intact filaments to a fully fragmented state (FIG. 23A). In contrast, when HaloTag® protein was localized to microtubules via β-tubulin-HaloTag® protein, microtubules remained largely intact over the same illumination period, with only partial fragmentation appearing at later time points (FIG. 23B).

Targeting HaloTag® protein to actin filaments using LifeAct-HaloTag® protein resulted in a gradual loss of F-actin signal and structure (FIG. 23C), while targeting HaloTag® protein to the mitochondrial matrix led to progressive mitochondrial damage, including swelling and fragmentation (FIG. 23D). These results demonstrate that the photochemical outcome of 561-nm illumination is strongly dependent on the subcellular position of the HaloTag-anchored photosensitizer, with robust intermediate filament fragmentation occurring specifically when the photosensitizer is localized to vimentin.

### Example 34: FilaBuster Induces Fragmentation of Multiple Cytoplasmic Intermediate Filament Systems

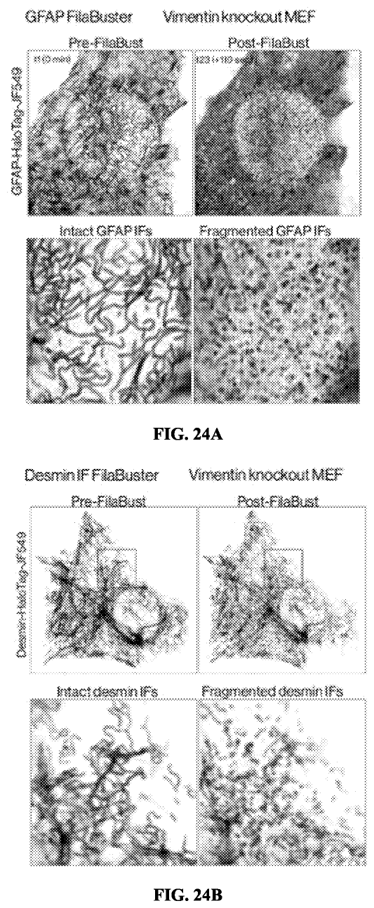

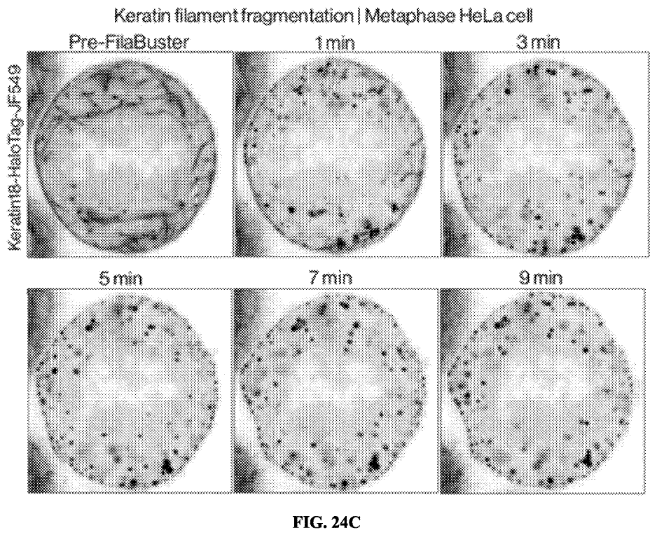

To assess whether photochemical fragmentation is specific to vimentin or represents a general property of cytoplasmic intermediate filaments, intermediate filament networks assembled in the absence of vimentin were examined (FIG. 24A-24C). Vimentin-knockout mouse embryonic fibroblasts expressing GFAP-HaloTag® protein exhibited an intact filamentous GFAP network prior to illumination that rapidly converted to punctate fragments following global activation (FIG. 24A). Similarly, desmin-HaloTag® protein expressed in vimentin-knockout cells formed a filamentous desmin network that fragmented into a dispersed punctate distribution after illumination (FIG. 24B).

Intermediate filament fragmentation was also examined in mitotic cells. In HeLa cells expressing keratin18-HaloTag® protein and imaged during metaphase, global illumination induced rapid loss of cortical keratin filaments and accumulation of bright keratin condensates over several minutes (FIG. 24C). Collectively, these experiments demonstrate that photochemical fragmentation can be applied to multiple cytoplasmic intermediate filament polymers, including vimentin, GFAP, desmin, and keratins, indicating that the phenomenon is not restricted to a single intermediate filament subtype.

### Example 35: Discussion of Studies

In these studies, FilaBuster is established as a generalizable strategy for rapid, specific, and spatiotemporally controlled IF disassembly in living cells. The approach consists of three components: (1) targeting a self-labeling protein (SLP) to native IFs, either through direct IF subunit fusion or IF-binding motifs; (2) covalent labeling with a cell-permeable photosensitizer SLP ligand; and (3) activation of the photosensitizer using controlled light exposure. Together, these steps enable optically triggered, highly localized IF destabilization with exceptional temporal precision, spatial control, and reproducibility.

Initial validation of the FilaBuster strategy focused on vimentin intermediate filaments (VIFs) as a well-characterized and structurally simple IF system. A vimentin-HaloTag® fusion protein was shown to co-assemble with endogenous vimentin filaments without perturbing native network architecture, as confirmed by fluorescence microscopy and cryo-electron tomography. Upon labeling with rhodamine-based HaloTag® ligands and brief illumination, rapid and complete disassembly of the VIF network was observed. These experiments established that photosensitizer activation at the filament surface is sufficient to drive acute IF disruption and provided a foundation for exploring the versatility and mechanistic boundaries of the approach.

Subsequent experiments demonstrated that FilaBuster is highly modular. Red-shifted photosensitizers, including JF₆₃₄, enabled efficient IF fragmentation using far-red excitation, expanding compatibility with standard microscope configurations and multiplexed imaging. A nanobody-based targeting strategy further broadened applicability by enabling photochemical disassembly of endogenous IFs without the need for direct IF subunit tagging or overexpression. Screening of a broad panel of HaloTag® ligands revealed that even conventional rhodamine dyes—despite lacking heavy-atom substitutions optimized for photosensitization—can drive IF disassembly when activated at sufficiently high photon flux, allowing a single ligand to function in both low-power imaging and high-power disassembly modes. Benchmarking across illumination regimes defined tunable thresholds for global versus focal IF fragmentation.

Further studies demonstrate that IF disassembly can be achieved under illumination conditions that do not elicit global phototoxicity. Dose-response analysis revealed a regime in which IF fragmentation occurs without detectable increases in cellular oxidative stress, ER calcium leakage, or mitochondrial depolarization. Under these threshold conditions, cells remained viable, adherent, and morphologically intact for extended periods following fragmentation and were able to re-establish a filamentous vimentin network over ~20 hours. These findings distinguish FilaBuster from conventional chromophore-assisted light inactivation approaches, which typically require orders-of-magnitude higher energy doses and are accompanied by widespread collateral damage.

Additional studies underscore the specificity of the FilaBuster mechanism. Fragmentation of IFs did not grossly perturb microtubule organization, mitochondrial morphology, or lysosome motility, indicating that acute IF disruption does not inherently destabilize other major cytoskeletal systems or organelles. Moreover, identical illumination conditions produced distinct outcomes depending on the subcellular localization of the HaloTag® protein-anchored photosensitizer. Robust filament fragmentation was observed when the photosensitizer was positioned on IFs, whereas targeting the same ligand to microtubules, actin filaments, or mitochondria resulted in qualitatively different or slower phenotypes. These results demonstrate that FilaBuster is not a nonspecific photochemical effect of light exposure, but rather a consequence of precise photosensitizer placement relative to a uniquely susceptible cytoskeletal substrate.

The mechanistic effectiveness of FilaBuster is consistent with emerging views of IFs as redox-sensitive polymers that function as cellular buffers against oxidative stress. IF networks are rich in oxidation-prone residues, extensively distributed throughout the cytoplasm, and capable of undergoing controlled subunit exchange and self-repair under mild stress. However, when localized oxidative damage exceeds a critical threshold, IFs disassemble abruptly, effectively sacrificing network integrity to protect other cellular components. FilaBuster exploits this intrinsic vulnerability by generating reactive species directly at the filament surface, converting a general biochemical susceptibility into a highly targeted and optically controlled disassembly mechanism.

Cryo-electron tomography revealed that HaloTag® protein moieties are positioned within nanometers of the filament surface and distributed along the filament length, placing photosensitizers well within the effective damage radius of short-lived reactive oxygen species. Because HaloTag® protein-labeled subunits co-assemble with untagged endogenous IF proteins, localized photochemical damage propagates efficiently through the filament lattice. Notably, this mechanism does not rely on modification of the canonical redox-sensitive cysteine residue in vimentin, but instead correlates with increased methionine oxidation at multiple sites, consistent with highly localized ROS generation and minimal cytoplasmic spillover.

Additional studies establish that FilaBuster is not limited to vimentin, but extends to multiple cytoplasmic IF classes. Robust photochemical fragmentation was demonstrated for GFAP, desmin, peripherin, and keratin-18 networks, including in cells lacking vimentin and in mitotic contexts. While the qualitative fragmentation dynamics differ between IF subtypes—reflecting differences in filament architecture, assembly rules, and post-translational regulation—the ability to induce selective disruption across diverse IF polymers highlights the generality of the approach. Mixed IF networks were also shown to disassemble cooperatively when a single IF subtype was targeted, consistent with the interdependent nature of cytoplasmic IF assemblies.

Together, these studies position FilaBuster as the first method to enable rapid, selective, and optically controlled disassembly of IFs in living cells with minimal off-target effects. By decoupling IF perturbation from chronic genetic manipulation or global chemical stress, FilaBuster provides a powerful tool for probing the immediate mechanical, organizational, and signaling functions of IFs in space and time. The ability to fragment IFs locally, reversibly monitor recovery, and apply the strategy across IF classes opens new avenues for dissecting the roles of intermediate filaments in cellular mechanics, organelle positioning, stress adaptation, and disease-relevant processes that have previously been inaccessible to experimental manipulation.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Bargagna-Mohan, P. et al. The tumor inhibitor and antiangiogenic agent withaferin A targets the intermediate filament protein vimentin. Chem. Biol. 14, 623-634 (2007).
2. Basu, A. et al. Vimentin undergoes liquid-liquid phase separation to form droplets which wet and stabilize actin fibers. Proc. Natl. Acad. Sci. U.S.A. 122, e2418624122 (2025).
3. Berr, A. L. et al. Vimentin is required for tumor progression and metastasis in a mouse model of non-small cell lung cancer. Oncogene 42, 2074-2087 (2023).
4. Binns, T. C. et al. Rational design of bioavailable photosensitizers for manipulation and imaging of biological systems. Cell Chem. Biol. 27, 1063-1072.e7 (2020).
5. Butkevich, A. N., Bossi, M. L., Lukinavičius, G. & Hell, S. W. Triarylmethane fluorophores resistant to oxidative photobluing. J. Am. Chem. Soc. 141, 981-989 (2019).
6. Carlier, M. F., Criquet, P., Pantaloni, D. & Korn, E. D. Interaction of cytochalasin D with actin filaments in the presence of ADP and ATP. J. Biol. Chem. 261, 2041-2050 (1986).
7. Chiu, C.-L., Clack, N. & the napari community. Napari: A python multi-dimensional image viewer platform for the research community. Microsc. Microanal. 28, 1576-1577 (2022).
8. Colakoğlu, G. & Brown, A. Intermediate filaments exchange subunits along their length and elongate by end-to-end annealing. J. Cell Biol. 185, 769-777 (2009).
9. Cremer, T. et al. RNF26 binds perinuclear vimentin filaments to integrate ER and endolysosomal responses to proteotoxic stress. EMBO J. 42, e111252 (2023).
10. De Brabander, M. J., Van de Veire, R. M., Aerts, F. E., Borgers, M. & Janssen, P. A. The effects of methyl (5-(2-thienylcarbonyl)-1H-benzimidazol-2-yl) carbamate, (R 17934; NSC 238159), a new synthetic antitumoral drug interfering with microtubules, on mammalian cells cultured in vitro. Cancer Res. 36, 905-916 (1976).
11. de Pablo, Y., Nilsson, M., Pekna, M. & Pekny, M. Intermediate filaments are important for astrocyte response to oxidative stress induced by oxygen-glucose deprivation and reperfusion. Histochem. Cell Biol. 140, 81-91 (2013).
12. Eckert, B. S. Alteration of intermediate filament distribution in PtK1 cells by acrylamide. Eur. J. Cell Biol. 37, 169-174 (1985).
13. Eibauer, M. et al. Vimentin filaments integrate low-complexity domains in a complex helical structure. Nat. Struct. Mol. Biol. 31, 939-949 (2024).
14. Eisenstein, F. et al. Parallel cryo electron tomography on in situ lamellae. Nat. Methods 20, 131-138 (2023).
15. Eriksson, J. E. et al. Introducing intermediate filaments: from discovery to disease. J. Clin. Invest. 119, 1763-1771 (2009).
16. Gan, Z. et al. Vimentin intermediate filaments template microtubule networks to enhance persistence in cell polarity and directed migration. Cell Syst. 3, 252-263.e8 (2016).
17. Goldman, R. D., Khuon, S., Chou, Y. H., Opal, P. & Steinert, P. M. The function of intermediate filaments in cell shape and cytoskeletal integrity. J. Cell Biol. 134, 971-983 (1996).
18. González-Jiménez, P. et al. Vimentin single cysteine residue acts as a tunable sensor for network organization and as a key for actin remodeling in response to oxidants and electrophiles. Redox Biol. 64, 102756 (2023).
19. Gorbachev, D. A., Staroverov, D. B., Lukyanov, K. A. & Sarkisyan, K. S. Genetically encoded red photosensitizers with enhanced phototoxicity. Int. J. Mol. Sci. 21, 8800 (2020).
20. Grimm, J. B. et al. A general method to improve fluorophores for live-cell and single-molecule microscopy. Nat. Methods 12, 244-250 (2015).
21. Grimm, J. B. et al. A general method to fine-tune fluorophores for live-cell and in vivo imaging. Nat. Methods 14, 987-994 (2017).
22. Grimm, J. B. et al. A general method to optimize and functionalize red-shifted rhodamine dyes. Nat. Methods 17, 815-821 (2020).
23. Grimm, J. B. et al. A general method to improve fluorophores using deuterated auxochromes. JACS Au 1, 690-696 (2021).
24. Grimm, J. B. et al. Optimized red-absorbing dyes for imaging and sensing. J. Am. Chem. Soc. 145, 23000-23013 (2023).
25. Grin, B. et al. Withaferin A alters intermediate filament organization, cell shape and behavior. PLOS One 7, e39065 (2012).
26. Griesser, E. et al. Dynamic posttranslational modifications of cytoskeletal proteins unveil hot spots under nitroxidative stress. Redox Biol. 44, 102014 (2021).
27. Gyoeva, F. K. & Gelfand, V. I. Coalignment of vimentin intermediate filaments with microtubules depends on kinesin. Nature 353, 445-448 (1991).
28. Hatz, S., Lambert, J. D. C. & Ogilby, P. R. Measuring the lifetime of singlet oxygen in a single cell: addressing the issue of cell viability. Photochem. Photobiol. Sci. 6, 1106-1116 (2007).
29. Hoelzel, C. A. & Zhang, X. Visualizing and manipulating biological processes by using HaloTag and SNAP-tag technologies. ChemBioChem 21, 1935-1946 (2020).
30 Hol, E. M. & Capetanaki, Y. Type III intermediate filaments Desmin, glial fibrillary acidic protein (GFAP), vimentin, and peripherin. Cold Spring Harb. Perspect. Biol. 9, (2017).
31. Jay, D. G. Selective destruction of protein function by chromophore-assisted laser inactivation. Proc. Natl. Acad. Sci. U.S.A. 85, 5454-5458 (1988).
32. Keppler, A. & Ellenberg, J. Chromophore-assisted laser inactivation of alpha- and gamma-tubulin SNAP-tag fusion proteins inside living cells. ACS Chem. Biol. 4, 127-138 (2009).
33. Kremer, J. R., Mastronarde, D. N. & McIntosh, J. R. Computer visualization of three-dimensional image data using IMOD. J. Struct. Biol. 116, 71-76 (1996).
34. Leung, C. L., Green, K. J., and Liem, R. K. H., Plakins: a family of versatile cytolinker proteins. Trends in Cell Biology, Volume 12, Issue 1, 37-45 (2002).
35. Lin, Y. et al. Toxic PR poly-dipeptides encoded by the C9orf72 repeat expansion target LC domain polymers. Cell 167, 789-802.e12 (2016).
36. Los, G. V. et al. HaloTag: a novel protein labeling technology for cell imaging and protein analysis. ACS Chem. Biol. 3, 373-382 (2008).
37 Lynch, C. D. et al. Endoplasmic spreading requires coalescence of vimentin intermediate filaments at force-bearing adhesions. Mol. Biol. Cell 24, 21-30 (2013).
38 Maier, J., Traenkle, B. & Rothbauer, U. Real-time analysis of epithelial-mesenchymal transition using fluorescent single-domain antibodies. Sci. Rep. 5, 13402 (2015).
39. Martínez-Cenalmor, P. et al. Oxidative stress elicits the remodeling of vimentin filaments into biomolecular condensates. Redox Biol. 75, 103282 (2024).
40. Mastronarde, D. N. Automated electron microscope tomography using robust prediction of specimen movements. J. Struct. Biol. 152, 36-51 (2005).
41. Mastronarde, D. N. & Held, S. R. Automated tilt series alignment and tomographic reconstruction in IMOD. J. Struct. Biol. 197, 102-113 (2017).
42. Matveeva, E. A. et al. Vimentin intermediate filaments protect mitochondria from oxidative stress. Biochemistry (Moscow) 4, 321-331 (2010).
43 Melak, M., Plessner, M., and Grosse, R. Actin visualization at a glance. Journal of Cell Science, Volume 130, Issue 3, 525-530 (2017).
44. Mónico, A. et al. Vimentin disruption by lipoxidation and electrophiles: Role of the cysteine residue and filament dynamics. Redox Biol. 23, 101098 (2019).
45. Na, N. et al. Mitochondrial reactive oxygen species are required for hypoxia-induced degradation of keratin intermediate filaments. FASEB J. 24, 799-809 (2010).
46. Onukwufor, J. O. et al. Quantification of reactive oxygen species production by the red fluorescent proteins KillerRed, SuperNova and mCherry. Free Radic. Biol. Med. 147, 1-7 (2020).
47. Paron, I. et al. A proteomic approach to identify early molecular targets of oxidative stress in human epithelial lens cells. Biochem. J. 378, 929-937 (2004).
48. Pérez-Sala, D. et al. Vimentin filament organization and stress sensing depend on its single cysteine residue and zinc binding. Nat. Commun. 6, 7287 (2015).
49. Prahlad, V. et al. Rapid movements of vimentin on microtubule tracks: kinesin-dependent assembly of intermediate filament networks. J. Cell Biol. 143, 159-170 (1998).
50. Rajfur, Z. et al. Dissecting the link between stress fibres and focal adhesions by CALI with EGFP fusion proteins. Nat. Cell Biol. 4, 286-293 (2002).
51. Renganathan, B. et al. Vimentin filament transport and organization revealed by single-particle tracking and 3D FIB-SEM. J. Cell Biol. 224, e202406054 (2025).
52. Ridge, K. M. et al. Methods for determining the cellular functions of vimentin intermediate filaments. Methods Enzymol. 568, 389-426 (2016).
53. Ridge, K. M., Eriksson, J. E., Pekny, M. & Goldman, R. D. Roles of vimentin in health and disease. Genes Dev. 36, 391-407 (2022).
54 Rothbauer, U. et al. Targeting and tracing antigens in live cells with fluorescent nanobodies. Nat. Methods 3, 887-889 (2006).
55. Sager, P. R. & Matheson, D. W. Mechanisms of neurotoxicity related to selective disruption of microtubules and intermediate filaments. Toxicology 49, 479-492 (1988).
56 Strnad, P., Windoffer, R. & Leube, R. E. Induction of rapid and reversible cytokeratin filament network remodeling by inhibition of tyrosine phosphatases. J. Cell Sci. 115, 4133-4148 (2002).
57. Takemeto, K. Optical manipulation of molecular function by chromophore-assisted light inactivation. Proc. Jpn. Acad. Ser. B 97, 197-209 (2021).
58. Takemoto, K. et al. Chromophore-assisted light inactivation of HaloTag fusion proteins labeled with eosin in living cells. ACS Chem. Biol. 6, 401-406 (2011).
59. Tinevez, J.-Y. et al. TrackMate: An open and extensible platform for single-particle tracking. Methods 115, 80-90 (2017).
60. Tour, O. et al. Genetically targeted chromophore-assisted light inactivation. Nat. Biotechnol. 21, 1505-1508 (2003).
61. Tran, Q. D. et al. Continuous self-repair protects vimentin intermediate filaments from fragmentation. bioRxiv 2024.09.02.610785 (2024).
62. Usman, S. et al. Impact of N-terminal tags on de novo vimentin intermediate filament assembly. Int. J. Mol. Sci. 23, 6349 (2022).
63. Valen, G. et al. Hydrogen peroxide induces endothelial cell atypia and cytoskeleton depolymerization. Free Radic. Biol. Med. 26, 1480-1488 (1999).
64. Viedma-Poyatos, A., de Pablo, Y., Pekny, M. & Pérez-Sala, D. The cysteine residue of glial fibrillary acidic protein is a critical target for lipoxidation and required for efficient network organization. Free Radic. Biol. Med. 120, 380-394 (2018).
65. Viedma-Poyatos, A., Pajares, M. A. & Pérez-Sala, D. Type III intermediate filaments as targets and effectors of electrophiles and oxidants. Redox Biol. 36, 101582 (2020).
66. Vitriol, E. A. et al. Enhanced EGFP-chromophore-assisted laser inactivation using deficient cells rescued with functional EGFP-fusion proteins. Proc. Natl. Acad. Sci. U.S.A. 104, 6702-6707 (2007).
67. Wagner, T. et al. SPHIRE-crYOLO is a fast and accurate fully automated particle picker for cryo-EM. Commun. Biol. 2, 218 (2019).
68. Wagner, T. et al. Two particle-picking procedures for filamentous proteins: SPHIRE-crYOLO filament mode and SPHIRE-STRIPER. Acta Crystallogr. D 76, 613-620 (2020).
69. Wagner, T. R. & Rothbauer, U. Nanobodies right in the middle: Intrabodies as toolbox to visualize and modulate antigens in the living cell. Biomolecules 10, 1701 (2020).
70. Weber, M. S. et al. Cellular and structural studies of eukaryotic cells by cryo-electron tomography. Cells 8, (2019).
71. Wong, I. Y. et al. Anomalous diffusion probes microstructure dynamics of entangled F-actin networks. Phys. Rev. Lett. 92, 178101 (2004).
72. Yan, P. et al. Fluorophore-assisted light inactivation of calmodulin involves singlet-oxygen mediated cross-linking and methionine oxidation. Biochemistry 45, 4736-4748 (2006).
73. Yang, J.-Y. & Yang, W. Y. Spatiotemporally controlled initiation of Parkin-mediated mitophagy within single cells. Autophagy 7, 1230-1238 (2011).
74. Yatsunami, J. et al. Vimentin is hyperphosphorylated in primary human fibroblasts treated with okadaic acid. Biochem. Biophys. Res. Commun. 177, 1165-1170 (1991).
75. Yoon, M. et al. Motile properties of vimentin intermediate filament networks in living cells. J. Cell Biol. 143, 147-157 (1998).
76. Zheng, Q. et al. Rational design of fluorogenic and spontaneously blinking labels for super-resolution imaging. ACS Cent. Sci. 5, 1602-1613 (2019).
77. Zheng, S. Q. et al. MotionCor2: anisotropic correction of beam-induced motion for improved cryo-electron microscopy. Nat. Methods 14, 331-332 (2017).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of inducing disassembly of an intermediate filament (IF) in a cell or tissue, comprising:
(a) expressing in, or introducing into, the cell or tissue a fusion protein comprising:
(i) a self-labeling protein derived from a haloalkane dehalogenase, and
(ii) an IF-targeting protein that either incorporates into IF polymers or binds specifically to IF proteins;
(b) administering to the cell or tissue a rhodamine- or fluorescein-derived photosensitizer modified with a ligand that specifically binds to the self-labeling protein, thereby associating the photosensitizer with the IF; and
(c) administering light to the cell or tissue under conditions effective to activate the photosensitizer and induce disassembly of the IF.

2. The method of claim 1, wherein the IF-targeting protein is an IF protein.

3. The method of claim 2, wherein the IF protein is selected from the group consisting of vimentin, desmin, glial fibrillary acidic protein (GFAP), peripherin, keratin 18, and nestin.

4. The method of claim 1, wherein the IF-targeting protein is a nanobody protein, an intracellularly expressible antibody or antibody fragment, a plectin-derived IF-binding domain, or a desmoplakin-derived IF-binding domain.

5. The method of claim 1, wherein the self-labeling protein specifically binds to a ligand having the following chemical structure:

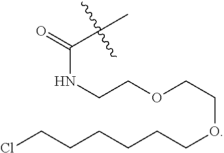 [SMILES: *C(=O)NCCOCCOCCCCCCCl]

6. The method of claim 1, wherein the fusion protein comprises the self-labeling protein positioned at the C-terminus of the IF-targeting protein.

7. The method of claim 1, wherein the fusion protein is expressed from a nucleic acid introduced into the cell or tissue.

8. The method of claim 1, wherein the fusion protein is introduced into the cell or tissue as a protein.

9. The method of claim 1, wherein the fusion protein is expressed via a viral vector.

10. The method of claim 1, wherein the fusion protein is expressed from a knock-in genomic locus encoding an endogenous IF protein.

11. The method of claim 1, wherein the light comprises laser illumination.

12. The method of claim 1, wherein the light is administered to a localized subcellular region of the cell.

13. The method of claim 1, wherein the cell is a mammalian cell.

14. The method of claim 1, wherein the tissue is a live tissue in an animal.

15. A kit, comprising:
(a) at least one vector comprising a nucleotide encoding a fusion protein comprising:
(i) a self-labeling protein derived from a haloalkane dehalogenase; and
(ii) an intermediate filament (IF)-targeting protein; and
(b) a rhodamine- or fluorescein-derived photosensitizer modified with a ligand that specifically binds to the self-labeling protein.

16. The kit of claim 15, further comprising one or more additional rhodamine- or fluorescein-derived photosensitizers having different excitation or emission wavelengths.

17. The kit of claim 15, wherein the IF-targeting protein is an IF protein.

18. The kit of claim 17, wherein the IF protein is selected from the group consisting of vimentin, desmin, glial fibrillary acidic protein (GFAP), peripherin, keratin 18, and nestin.

19. The kit of claim 15, wherein the IF-targeting protein is a nanobody protein, an intracellularly expressible antibody or antibody fragment, a plectin-derived IF-binding domain, or a desmoplakin-derived IF-binding domain.

20. The kit of claim 15, wherein the self-labeling protein specifically binds to a ligand having the following chemical structure: 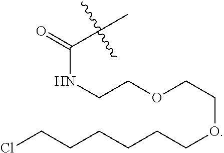 [SMILES: *C(=O)NCCOCCOCCCCCCCl]


SEQUENCE LISTING (4 sequences)

SEQ ID NO: 1 - protein, 296 aa, synthetic construct
AEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 2 - protein, 296 aa, synthetic construct
AEIGTGFPFDPHYVEVLGERMHYVDVGPRDGTPVLFLHGNPTSSYVWRNIIPHVAPTHRTIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNVKAVDIGPGLNLLQEDNPDLIGSEIARWLSTLEISG

SEQ ID NO: 3 - protein, 6 aa, synthetic construct
GSGSGS

SEQ ID NO: 4 - protein, 22 aa, synthetic construct
RSLGGGGSGGGGSGGGGSGGGG